SEQUENCE LISTING (112 sequences)

SEQ ID NO: 1 - RNA, 144 nt, Homo sapiens
gacccugggccuccaccaccauccccucuggugggccugaagccacugcagcugcuggagaucaaggcucgggggcgcuuuggcugugucuggaaggcccagcucaugaaugacuuuguagcugucaagaucuucccacuccag

SEQ ID NO: 2 - RNA, 144 nt, Homo sapiens
gacaagcagucguggcagagugaacgggagaucuucagcacaccuggcaugaagcacgagaaccugcuacaguucauugcugccgagaagcgaggcuccaaccucgaaguagagcuguggcucaucacggccuuccaugacaag

SEQ ID NO: 3 - RNA, 149 nt, Homo sapiens
ggcucccucacggauuaccucaaggggaacaucaucacauggaacgaacugugucauguagcagagacgaugucacgaggccucucauaccugcaugaggaugugcccuggugccguggcgagggccacaagccgucuauugcccacag

SEQ ID NO: 4 - RNA, 115 nt, Homo sapiens
ggacuuuaaaaguaagaauguauugcugaagagcgaccucacagccgugcuggcugacuuuggcuuggcuguucgauuugagccagggaaaccuccaggggacacccacggacag

SEQ ID NO: 5 - RNA, 139 nt, Homo sapiens
guaggcacgagacgguacauggcuccugaggugcucgagggagccaucaacuuccagagagaugccuuccugcgcauugacauguaugccaugggguuggugcugugggagcuugugucucgcugcaaggcugcagacg

SEQ ID NO: 6 - RNA, 131 nt, Homo sapiens
gacccguggaugaguacaugcugcccuuugaggaagagauuggccagcacccuucguuggaggagcugcaggaggugguggugcacaagaagaugaggcccaccauuaaagaucacugguugaaacacccg

SEQ ID NO: 7 - RNA, 195 nt, Homo sapiens
ggccuggcccagcuuugugugaccaucgaggagugcugggaccaugaugcagaggcucgcuuguccgcgggcuguguggaggagcgggugucccugauucggaggucggucaacggcacuaccucggacugucucguuucccuggugaccucugucaccaauguggaccugcccccuaaagagucaagcaucuaa

SEQ ID NO: 8 - RNA, 11782 nt, Homo sapiens
ggcucggcuccguggccgccgcucaggagccauuuuggacucgguucagcuccccucccccccaccccuccccccguucauggccccuccggacucggccccugcgcccggggcccgggcccagccccgccgcgcuaugccugagucgggcgcgccccggcccgugccccgccgccgccccccggcccccgcgucgccccggagcccgggccgcagccugcgccgcccgcagcggcccugagcccggccccgccgaccggcccuuggagcccgaacgcugcucggggacgaaggcgcaggaagcgcgcagggaacgagaccgaaggaaggagcgggaaggagagcgcagccgccgccuggcccugcgcgccccgggagcgccgugcggcccugcccgcgggcuccgggugugcgcggggcggcgccgcggaacaugacggcgcccuggguggcccucgcccuccucuggggaucgcugugcgccggcucugggcguggggaggcugagacacgggagugcaucuacuacaacgccaacugggagcuggagcgcaccaaccagagcggccuggagcgcugcgaaggcgagcaggacaagcggcugcacugcuacgccuccuggcgcaacagcucuggcaccaucgagcucgugaagaagggcugcuggcuagaugacuucaacugcuacgauaggcaggaguguguggccacugaggagaacccccagguguacuucugcugcugugaaggcaacuucugcaacgaacgcuucacucauuugccagaggcugggggcccggaagucacguacgagccacccccgacagcccccacccugcucacggugcuggccuacucacugcugcccaucgggggccuuucccucaucguccugcuggccuuuuggauguaccggcaucgcaagccccccuacggucauguggacauccaugaggacccugggccuccaccaccauccccucuggugggccugaagccacugcagcugcuggagaucaaggcucgggggcgcuuuggcugugucuggaaggcccagcucaugaaugacuuuguagcugucaagaucuucccacuccaggacaagcagucguggcagagugaacgggagaucuucagcacaccuggcaugaagcacgagaaccugcuacaguucauugcugccgagaagcgaggcuccaaccucgaaguagagcuguggcucaucacggccuuccaugacaagggcucccucacggauuaccucaaggggaacaucaucacauggaacgaacugugucauguagcagagacgaugucacgaggccucucauaccugcaugaggaugugcccuggugccguggcgagggccacaagccgucuauugcccacagggacuuuaaaaguaagaauguauugcugaagagcgaccucacagccgugcuggcugacuuuggcuuggcuguucgauuugagccagggaaaccuccaggggacacccacggacagguaggcacgagacgguacauggcuccugaggugcucgagggagccaucaacuuccagagagaugccuuccugcgcauugacauguaugccaugggguuggugcugugggagcuugugucucgcugcaaggcugcagacggacccguggaugaguacaugcugcccuuugaggaagagauuggccagcacccuucguuggaggagcugcaggaggugguggugcacaagaagaugaggcccaccauuaaagaucacugguugaaacacccgggccuggcccagcuuugugugaccaucgaggagugcugggaccaugaugcagaggcucgcuuguccgcgggcuguguggaggagcgggugucccugauucggaggucggucaacggcacuaccucggacugucucguuucccuggugaccucugucaccaauguggaccugcccccuaaagagucaagcaucuaagcccaggacaugagugucuguccagacucaguggaucugaagaaaaaaggaaaaaaaguuguguuuuguuuuggaaaucccauaaaaccaacaaacacauaaaaugcagcugcuauuuuaccuugacuuuuuauuauuauuauuauaauuauuauaauuauuauuauuaauauuauuuuuuggauuggaucaguuuuuaccagcauauugcucuacuguaucacaaacagcggacacgucagcaggcguugaggugcugagcuguggaugcagaaccagcgccaugcugaagagccucagccaccuccuguccuuugggauucguuuuucccgcuuucucuuuguuugucgucucagaaucugugacacaaagaaacccaucuccugucuuaggaaaccuaaugcugcaaacucuaccuagaggaaccuuugaagacuguuacauaagaacauaccuuccucagaagaggaguuuccucugcccucugcccuucuccccugccucccucccuccccuccuuuuauuuuguuuuagugagcuuaagaaacagcagaugugucuuucacggaucuaacggguguuguccugaucgagaaaaaaacugggaugagaaugguuuggacuggaguuggaaggggaggacgguacuggggguaggguuuggaacagagcuacacuggacucgggcacauucggagcagcauccuuuaguauggaggcuacuucucagguaaccaggaauugaggggaaggaccuuguggaggccgagcauuaacagcaagagcgggguuuggagaaagucugagauugggugcagcccugacuuaccugcuggcccugaccaguuucuuuucacuaacuuggccuugggcauaggaugaaacauuuuuucugcccuaauuuuaaaacuaggugaggguagaaucaucacagguuaggaauacauucuucauaagacacgaugcuguaaauacccuuaauggacgaaaaguugaaauacuuuuguuuccucuuggagcaguucagggaaaugcccacaggggauuguccugcacagauagggcaagaggauuuccuggguggagucugccaaggccugccucgcuggggaccccagaguccugcaccucugguuccgccccagguggugacauuacuguccccguucuguggcucguggacaagacuuucuccagaccccuuaaagugguacauauucuaaaaaacuguuuuucuauuaugccauaaccuugcucuagucagugaauguuccuaaugcugcuguuucaacauuugaauucuuuuuaauuuaugaaacaugcuaaauuuuuuuuuucaaacaaaacacacacauccacauauacacaugcuucgcuauguggcuuccaagguuuaaauuuugaaaaguaaaagaauuaaaacuucacgaccacagaucaccucaaaccagaaauaccucagaauuuucuacuuauguaagguuuauuauauauuuuguuaguuguguugucuuguaguaaguauauuuuaauguaaguuggcuuuugugacaaggaaguuuaaaagaaauagagaaaaagaaaaaaguuugcaucuucuagggagugcuaccauuuuuguuugauaacgcccccuuguaaauaauugucaucaacuguagguuggcugucugggccaagucugggcauuuaucagucuuguuugugaaggcuuuuccuucugguuucuuuagaucauuuuauuuaaaaacagugcaucucuucaucgugaggguaggcaaggcgggggccguggggagagguugaccugggugagaacugaagaggccgccuccucuuggguuguuuggagcuucacauguaauucacauguaacauguaacuugaucggucaguguucagaaugacaaguaaccccgcuuaaacuugguagaaggauggcccuuagaccugaauggggugauuuuacuugggauuuaacuucuucagcaaauuaacagcaacguuggaagagaucuguggcgccucugugaagcacaccgugacucaggccagucuuuuagugcagcgugucugggagugaaggguuuugcccuugcuggucuuggaguccacagugugaggggcacugcacaugccugggcaucuaccuagugugcuauguucagugucuggggcuuacugccccgggguccuuuccucuggguguuggggcacagggugcuaugggaggcccauuugcuucccucucggagcucaguuuuugcuucaugggucaaaaugugggcuggccaagugguuacaggaacaggguuucgguaagcuauguugucuuuuuuuuuuuuuuuuuuuuuuuuuuuaaugguuugauuuugugcugugguauuuuuuuucccuuagaauaauuuuuaauggcaaaacaggccuuacagcaguugcuuuucuuuaccauuuauuucuuuaagaagcuuuaaaauauuuauugaaaagugccauaucuaauuucuuuagcuuucgccucaggcagugcaggcaucuuuacuuuucauccucagaagaaacaaacgacuaacaaauguagcaaauuuacugcaggaauaguuaggucaugauacuaccugaacacuaaaccccagccucuuuguuugguuuuaguuccucugggugguuuuucuuuugugugcuggcuugauucuugugagaaguuuugaccuggccaagggaggguugagccaugguucuggugugggacuuugcggucaagacacaguacagacaggucaggccugcgugccuuuucucuggguggccuccccguuaggcccaccguacgcucagccacuauagugucccuguggggccuugccaucagauugugugucaggagaugguaccuuuuugguguggcuggggaggagugugguccaugccaguucuuugggcuucaggccacucuuccccucaugcugugguguaaagugcacccaucaggugguauaucugguucugauggcaagaagaaggugggggaucuccuuauagggcaugggucuaggagcacagaugggccuuuugccccggguaaaugcuugucuguuugcugucauguguucuuugaggagugagccaucucgagcccugcuuugaauuuacugggucauagagccucugccugugcucuuuuccauaaugacuucaugugacaugcacuuuuggugggcucagauaauugguuucuuuuuguuuuugaccucaggcucuguggcagacuggggaaaauggggccuggcaucauuuucccugucaaugggaggggcuguuccaugcagggugggaggggaccaaguuagcagagaguagccaaggauccuugcuucuuccuuucuagugugcugucauccaagcaggcuccuggcuguagggaugggccuuggggaagaaucuucuuugaaagcaucuaugauaacugagaagucaucccuaguuggagaaauccaguaaugagcagaaggaggaagcaagugaggacagaggccauuguauuacagugucacgcagagggcccucaaugauggggcauuggggaaggcuguagacauagucaucagaacauccuggccuggcauaagcuggguuuucuccugggaccauugguccucagcaggaguucuuugcaugaguugcucaggggcaagggcugcaagugggcugugcuuaggagaaagugacaccuggcagugagggaagauggugagcauuauuagccuuuguuguccagcauggccuucuuguccugucugcucuggagaggagccugugggaccaguccugccuggggagggcauacccacacgugccagcugauucugacucugaauacaucauguccggacuuggggguguuucugcagaaaaaggagguuguuuuucagccuugaacaucuucaggaggauagagacucuugcucacauauucuuagcaaagggaagggucucucaucuccaggccacagagauaguucuuccauugcccuaagaggcuaggcuaacccucuugacauaacuuagacagcaaagcacuucauccuguaguugggcucugucaccuuucucuucaguuggccacauucucguuuccuccauccugcuaugcuuugugugcucgggcugugugugggguuuuucccugguggaaggaagcccagcuguguauugaauguccuucauguguuguguguggcucagaaagccugucacuuggccccugugcucugagccgugaggguggggagguggcuguuccauuaaagugggaguauuggauggcccucuugaaacuagaauuuugccuuuuuuaguaugcaguauaaaguuuccagcaucuauugguaacacaaagauuugcugguuuuuaaaauaauacaguaagcauaaguauguaaguuuuuagaauugguacuagaaguuggacagcuaguuauucucgagaacuuuauuucacuagaaaaauauacuaauuggaaagcaguuuccaggaguuaacucaguuuaauuuucagucucaguuauuuuagccuguugaguuuuugauggcacaccuuuggagagauggccacgccugauucccauuucaggggcaucagaccauaccuuuuuaagaagcuccgugaaucuagucaucuacccuucauccugggcgaacagccaaaaagagaaggggacaaggugucuuuuucuccuucucacuggggugacaugaauucuuuuaguuaauggcuguuugcaaauucuaaacuaaugaaauacuuagcagcuaacauguucaaucuaguaaugaugaguuuaaaucucaauugacaguaauguuuuagauaaacaggcccaguaauucaguugaugaacuguauaucuucucagucuagauuuguaaauguuuaaugaauucaggguuauaagcauaguucuuuaaguaagauuccagauaguugauuugcaaccagcagucuaccuaugaauguaucccaaaccuuuagaagauuggaaaagauuuuugaaauaaugauuuaguuuuguaggaaaaacacccccuugaaaauuaauucgguugacccaguaacauuuuuuaaaacaauugguggcuccaaaaggccugccaacaaagaaaaguccaaauuaucuagugggacauuuugaauguuuuauguuuauuuuggguccacuguaaacuuugguucaaaaaagaauuugaauuuaaagaauuuaccauuauuuaaauuauuaccaaguuuuuacauuuucaugaugguauuuuccagguaugaaugaaacaugacuuuuugauugugguacuuccuguauccccuguagugccaaaaccagugauacuuuauuugcuccuauggcagcucauagagguaaccgaagugauuuuuccucaguaauugaaacacauauucucuaaaugccaauguguggugaugggcccugcacugccuucauuucucuagggcagugucuuuggauugucuagggccuagguaauucugagaacuacuguaaaccaaccacagggcacuaaagcaauguacacaccacucuuuguguguauggaagggguuauauaaaccugggcuaugcuggacaucuacagaagaguauuacauucacuugcaaaguuuacauuuuugagcucacaguuaugaaaaauaugacccacaaguuuuucaggcaggugaggaugggucuucuugcaaaugcaugaguucugucuugaguccugggaacuucucuguugguugagugugggcucauucccugacucuccuaaucauguuugcgucagaauguuagcauuguaaauaaaagaauagguuguauaauagauacacaacacuugaaacuuuacuuuaaaaaaaucgauaguucuacauauauauuuaguuauaucacuugacagauuucuucuacacaguguggagauuguuuuauaccacagauuauuuuuauaaaguuagugaauuugaaugauuuuguaaucagagcuaaugagcuuuaccuuucaagagaaacguacacuggagcaugagugguguggaacuuuuacuuaguguuuauauggauucuugugauacacuggcagacuggagucaauuugcgggucuuuuuuggccaaaacuccacuugugguuguguaggacagugauauucagcucagcuucuuguggauugggaggagagagggccugcaauguguuuuacauuggugcuuccuccugagauuucuguugaacaaaggguucugaggucaaaaauuaguuuguaagccuuugccauaggacauagucaugugagaguguuugggggaacagaaauuguauaggggugccuauuggggugggaugggacucgaauaagauucagguacaaaaacuuugaaaugagaaucuggugguuugaguaauccaccagacugaauuaucuaagaucacauuauccagguuggggggcagaauuacccaguuaaguaauuguucagaaaaguggggaggguggcauguggaugcagugauccaauuaaauggagagcugccaggcacauuuuguccucucuggucagugagaaugguuggguuggcucgcugcuucaaucuguggaaucagccaggagcccagugaggaagcucagaaccccaguaacagcagagcaucuuucagauagcuccagaguuuuccugcuuuucugaggaagcucagcaucacugccacaauacggaaaguggucuucauuuuagccuauuuauuuuuaggcagagaguggaugguuauuugugugggacuuuugguggcgauauauaaugaauaauuaaguuaauuucugguaugcauaauggccaguccugaggcccagcugaagaccugucccccagacccugcccgcuggcuucaggcugcugcuucuagacagaggugcacuggacgggauaguuuuaucaagagaaucccuaaugugucauuuuaaaccagcugugcuuuuuauucauucugguugagcguauagguuuacacuuuacccuuuuuauacuuggaauaaauuuaguuccagcagaucuaguagcacuccagaaaccaaccccaucuguuccccauaaaaagaacauuuucucugcucuccagccacgugucuuggaauguaauucuguugugccuuuguuuuuaucacucucuucgccccaaaagcaacugcuguaagcuuuuuucuacuugucuuuucuaguccccaaccucuaccuuuuuccuuuuucccagcccuaauuucuggaugcacuucugugauccagguauuuuaagaaccaguuaccucagaccucauguugaacagugucgccaucuggguccucuugauacugcagacuuuuaacguacacaugcaggaacccugcugagcgugggcacuuguuuuaaagcaaaacucuucccaaggacugaagaaagggcuucuggcaagcucgucauggcauuguggugggaugggucuagagugucaucugaauggugcuuccuguguuccucuuugaauucugccauuuucaguauucuugugugucugaauaggcaaagcgauuuaauuggcuggucuugcacgcaaauuaguuccaaagauaagcucuuuguaacacauuuccagucgcuaaugcucaaauguagaacauuccuuuaaauggcaggauaaaaaacccacuauccaccauagugcauuuugggaagaugucuguagcauauguugcugugaaauuaggccuugugggauauggcuguuugucauuuugauguauuuuaaauaaauauauauauuuuuuaaagagccuuuuuuaccaguucaaaaaguuuaauuaaccagcagucaccgcaucugaauuuuugucucuggggcauagauggcagaccaagauuaaaagugguaacucagcuauacgagcaugggcuaccuuccugggcucuccugcaguccuguagaccugcuguuccgcagaccaugggacacaaggucaguguguucccagugagggucccaagucagucaucuuaaguguuuguucucugccccauucaguggacuguugacuucagucccugcaagugcuuuagcccgagugggguuuucucagagcacugccacgaguuaaguguguguuuagccaaauaauuucuccguaagggaaaaaugcagucacccaaauuuuaccaacaaugacagagaugagaguagaaaagauuaggcaacaucugaguuuuaacuugaaaaguguccaagucaucaugaaaggccgacugggagcaagugauuauuagagauucuucaggagaccucaucugaaaauguuaagacugccagugagggaaggaauuguuaaaaugccagcggcuuuuuuuuccucuuuuuuucuguaauucuguaaaaaugcagagaaaguugagugguacuucagaauugagggagaggguuaccgcagaguagaaauauauuucuagauuucaguuccacaccacaaauccacaacaaugccauuuuucaacuguacaaaaaucugcuuaugaacuggacaugaucuuaaugguagugucaaaggccaaguuuuucaccuguuaauauuuuuccacauuuguccuugaaucugaauaacuuuauacaguacuguaaauuuaacuuacaucgaguuuguugucaauucuuaugaaaagagcuuucugcauguaacacauacgguuaaagaacacagcaaaggacaaaauuugcaggaacaguuuuggaaccaacagaaaaugucaccuuuuauuugccaucuuauauauaucuaucaguuuuaccagcuacuucuaaauuuguacauuauuuguaagggaaagaaggaaaacccuaagacuugucuaacuuaguggagaauguguguguugggcuuaggauggauagcuaagucuuauugagcuguguuaccuaacuuguauauaaaaauuguaauuaaaaguuuggguucaccuguuucucacaguuuaaaaugaugaguaauugcaaacucuggaaaugugacuaguauaugauuuaaggcuguagaagcaaggaagcucuuucaagugcuaaaacuaaagacuucuaguuuuuggcucaaauaaguacuguuuguauaccaggauaugugagauguaaauguaguaggucacuuuucacccuuguagcuauaaaauaaaaauuuuguagaacagaaauagcuuguacuacugaauuaacaaaaguuauacuaaaguaucauguuuaaaaaaaauauauauauauauacagaguuaagcuuguugcuguuacccugucuggauuugaaaagugugcugauuuauauauauauauuacacacacacacacacacacacacacacacacacacacacacacacacacacacacacacacacacacacacauacaccuaaaauggccuaaagcagacauccauguaauuacaguugcaaaaugaaaacauuuuggaaagaacauuguaucauaguucauucauuugcaguggaucuuuguuccuuuuuacugugguaauuuuagaaaugagugucaaguuugaaauuagaucugcuaaguugggguuuugcugcuugaacucugcacuggguccucaaauaaaccgaugugaauguaguuuuuucccccugugugaagaagcaguuacaccccaacaauaggaggaaaaaucuagaacuauuucaaguuuuaucuuuuuguauaugaaaauaaaauaauaauaaaacaa

SEQ ID NO: 9 - RNA, 1065 nt, Homo sapiens
auguaccggcaucgcaagccccccuacggucauguggacauccaugaggacccugggccuccaccaccauccccucuggugggccugaagccacugcagcugcuggagaucaaggcucgggggcgcuuuggcugugucuggaaggcccagcucaugaaugacuuuguagcugucaagaucuucccacuccaggacaagcagucguggcagagugaacgggagaucuucagcacaccuggcaugaagcacgagaaccugcuacaguucauugcugccgagaagcgaggcuccaaccucgaaguagagcuguggcucaucacggccuuccaugacaagggcucccucacggauuaccucaaggggaacaucaucacauggaacgaacugugucauguagcagagacgaugucacgaggccucucauaccugcaugaggaugugcccuggugccguggcgagggccacaagccgucuauugcccacagggacuuuaaaaguaagaauguauugcugaagagcgaccucacagccgugcuggcugacuuuggcuuggcuguucgauuugagccagggaaaccuccaggggacacccacggacagguaggcacgagacgguacauggcuccugaggugcucgagggagccaucaacuuccagagagaugccuuccugcgcauugacauguaugccaugggguuggugcugugggagcuugugucucgcugcaaggcugcagacggacccguggaugaguacaugcugcccuuugaggaagagauuggccagcacccuucguuggaggagcugcaggaggugguggugcacaagaagaugaggcccaccauuaaagaucacugguugaaacacccgggccuggcccagcuuugugugaccaucgaggagugcugggaccaugaugcagaggcucgcuuguccgcgggcuguguggaggagcgggugucccugauucggaggucggucaacggcacuaccucggacugucucguuucccuggugaccucugucaccaauguggaccugcccccuaaagagucaagcaucuaa

SEQ ID NO: 10 - DNA, 1539 bp, Homo sapiens
atgacggcgccctgggtggccctcgccctcctctggggatcgctgtgcgccggctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgaacgcttcactcatttgccagaggctgggggcccggaagtcacgtacgagccacccccgacagcccccaccctgctcacggtgctggcctactcactgctgcccatcgggggcctttccctcatcgtcctgctggccttttggatgtaccggcatcgcaagcccccctacggtcatgtggacatccatgaggaccctgggcctccaccaccatcccctctggtgggcctgaagccactgcagctgctggagatcaaggctcgggggcgctttggctgtgtctggaaggcccagctcatgaatgactttgtagctgtcaagatcttcccactccaggacaagcagtcgtggcagagtgaacgggagatcttcagcacacctggcatgaagcacgagaacctgctacagttcattgctgccgagaagcgaggctccaacctcgaagtagagctgtggctcatcacggccttccatgacaagggctccctcacggattacctcaaggggaacatcatcacatggaacgaactgtgtcatgtagcagagacgatgtcacgaggcctctcatacctgcatgaggatgtgccctggtgccgtggcgagggccacaagccgtctattgcccacagggactttaaaagtaagaatgtattgctgaagagcgacctcacagccgtgctggctgactttggcttggctgttcgatttgagccagggaaacctccaggggacacccacggacaggtaggcacgagacggtacatggctcctgaggtgctcgagggagccatcaacttccagagagatgccttcctgcgcattgacatgtatgccatggggttggtgctgtgggagcttgtgtctcgctgcaaggctgcagacggacccgtggatgagtacatgctgccctttgaggaagagattggccagcacccttcgttggaggagctgcaggaggtggtggtgcacaagaagatgaggcccaccattaaagatcactggttgaaacacccgggcctggcccagctttgtgtgaccatcgaggagtgctgggaccatgatgcagaggctcgcttgtccgcgggctgtgtggaggagcgggtgtccctgattcggaggtcggtcaacggcactacctcggactgtctcgtttccctggtgacctctgtcaccaatgtggacctgccccctaaagagtcaagcatctaa

SEQ ID NO: 11 - protein, 512 aa, Homo sapiens
MTAPWVALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 12 - DNA, 24 bp, Artificial
  Synthetic nucleic acid
cgagccttgatctccagcagctgc

SEQ ID NO: 13 - DNA, 24 bp, Artificial
  Synthetic nucleic acid
ccccgagccttgatctccagcagc

SEQ ID NO: 14 - DNA, 24 bp, Artificial
  Synthetic nucleic acid
tcatgagctgggccttccagacac

SEQ ID NO: 15 - DNA, 24 bp, Artificial
  Synthetic nucleic acid
ggagtgggaagatcttgacagcta

SEQ ID NO: 16 - DNA, 24 bp, Artificial
  Synthetic nucleic acid
ctggagtgggaagatcttgacagc

SEQ ID NO: 17 - DNA, 24 bp, Artificial
  Synthetic nucleic acid
acctggagtgggaagatcttgaca

SEQ ID NO: 18 - DNA, 24 bp, Artificial
  Synthetic nucleic acid
ctcaccttgtcatggaaggccgtg

SEQ ID NO: 19 - DNA, 24 bp, Artificial
  Synthetic nucleic acid
gatgttccccttgaggtaatccgt

SEQ ID NO: 20 - DNA, 24 bp, Artificial
  Synthetic nucleic acid
cagttcgttccatgtgatgatgtt

SEQ ID NO: 21 - DNA, 24 bp, Artificial
  Synthetic nucleic acid
ctacatgacacagttcgttccatg

SEQ ID NO: 22 - DNA, 24 bp, Artificial
  Synthetic nucleic acid
tatgagaggcctcgtgacatcgtc

SEQ ID NO: 23 - DNA, 24 bp, Artificial
  Synthetic nucleic acid
ctcatgcaggtatgagaggcctcg

SEQ ID NO: 24 - DNA, 24 bp, Artificial
  Synthetic nucleic acid
agggcacatcctcatgcaggtatg

SEQ ID NO: 25 - DNA, 24 bp, Artificial
  Synthetic nucleic acid
gtgaggtcgctcttcagcaataca

SEQ ID NO: 26 - DNA, 24 bp, Artificial
  Synthetic nucleic acid
ctgtgaggtcgctcttcagcaata

SEQ ID NO: 27 - DNA, 24 bp, Artificial
  Synthetic nucleic acid
cacggctgtgaggtcgctcttcag

SEQ ID NO: 28 - DNA, 24 bp, Artificial
  Synthetic nucleic acid
ccaagccaaagtcagccagcacgg

SEQ ID NO: 29 - DNA, 24 bp, Artificial
  Synthetic nucleic acid
ggaggtttccctggctcaaatcga

SEQ ID NO: 30 - DNA, 24 bp, Artificial
  Synthetic nucleic acid
cccctggaggtttccctggctcaa

SEQ ID NO: 31 - DNA, 24 bp, Artificial
  Synthetic nucleic acid
cgtgggtgtcccctggaggtttcc

SEQ ID NO: 32 - DNA, 24 bp, Artificial
  Synthetic nucleic acid
cctgtccgtgggtgtcccctggag

SEQ ID NO: 33 - DNA, 24 bp, Artificial
  Synthetic nucleic acid
caatgcgcaggaaggcatctctct

SEQ ID NO: 34 - DNA, 24 bp, Artificial
  Synthetic nucleic acid
gcatacatgtcaatgcgcaggaag

SEQ ID NO: 35 - DNA, 24 bp, Artificial
  Synthetic nucleic acid
cagcatgtactcatccacgggtcc

SEQ ID NO: 36 - DNA, 24 bp, Artificial
  Synthetic nucleic acid
cctgcagctcctccaacgaagggt

SEQ ID NO: 37 - DNA, 20 bp, Artificial
  Synthetic nucleic acid
tgctacgataggcaggagtg

SEQ ID NO: 38 - DNA, 20 bp, Artificial
  Synthetic nucleic acid
agcaggttctcgtgcttcat

SEQ ID NO: 39 - DNA, 20 bp, Artificial
  Synthetic nucleic acid
catgtggacatccatgagga

SEQ ID NO: 40 - DNA, 20 bp, Artificial
  Synthetic nucleic acid
gagacacaagctcccacagc

SEQ ID NO: 41 - DNA, 20 bp, Artificial
  Synthetic nucleic acid
tctattgcccacagggactt

SEQ ID NO: 42 - DNA, 19 bp, Artificial
  Synthetic nucleic acid
gagcctctgcatcatggtc

SEQ ID NO: 43 - DNA, 24 bp, Artificial
  Synthetic nucleic acid
gctgaagatctcccgttcactctg

SEQ ID NO: 44 - DNA, 24 bp, Artificial
  Synthetic nucleic acid
tagcaggttctcgtgcttcatgcc

SEQ ID NO: 45 - DNA, 24 bp, Artificial
  Synthetic nucleic acid
ccacggcaccagggcacatcctca

SEQ ID NO: 46 - DNA, 24 bp, Artificial
  Synthetic nucleic acid
tcagccagcacggctgtgaggtcg

SEQ ID NO: 47 - DNA, 24 bp, Artificial
  Synthetic nucleic acid
aaagtcagccagcacggctgtgag

SEQ ID NO: 48 - DNA, 24 bp, Artificial
  Synthetic nucleic acid
ccagcacggctgtgaggtcgctct

SEQ ID NO: 49 - DNA, 24 bp, Artificial
  Synthetic nucleic acid
gtttccctggctcaaatcgaacag

SEQ ID NO: 50 - DNA, 24 bp, Artificial
  Synthetic nucleic acid
cgctcttcagcaatacattcttac

SEQ ID NO: 51 - DNA, 24 bp, Artificial
  Synthetic nucleic acid
aggtcgctcttcagcaatacattc

SEQ ID NO: 52 - DNA, 24 bp, Artificial
  Synthetic nucleic acid
agccaaagtcagccagcacggctg

SEQ ID NO: 53 - DNA, 24 bp, Artificial
  Synthetic nucleic acid
cggctgtgaggtcgctcttcagca

SEQ ID NO: 54 - DNA, 24 bp, Artificial
  Synthetic nucleic acid
gccaagccaaagtcagccagcacg

SEQ ID NO: 55 - DNA, 24 bp, Artificial
  Synthetic nucleic acid
tcgaacagccaagccaaagtcagc

SEQ ID NO: 56 - DNA, 24 bp, Artificial
  Synthetic nucleic acid
gtgtcccctggaggtttccctggc

SEQ ID NO: 57 - DNA, 23 bp, Artificial
  Synthetic nucleic acid
caagccaaagtcagccagcacgg

SEQ ID NO: 58 - DNA, 24 bp, Artificial
  Synthetic nucleic acid
gttacctgtccgtgggtgtcccct

SEQ ID NO: 59 - DNA, 24 bp, Artificial
  Synthetic nucleic acid
ggctcaaatcgaacagccaagcca

SEQ ID NO: 60 - DNA, 24 bp, Artificial
  Synthetic nucleic acid
tccgtgggtgtcccctggaggttt

SEQ ID NO: 61 - DNA, 24 bp, Artificial
  Synthetic nucleic acid
ggtcgctcttcagcaatacattct

SEQ ID NO: 62 - DNA, 24 bp, Artificial
  Synthetic nucleic acid
attcttacttttaaagtccctgtg

SEQ ID NO: 63 - DNA, 24 bp, Artificial
  Synthetic nucleic acid
tacattcttacttttaaagtccct

SEQ ID NO: 64 - DNA, 24 bp, Artificial
  Synthetic nucleic acid
tgaggtcgctcttcagcaatacat

SEQ ID NO: 65 - DNA, 24 bp, Artificial
  Synthetic nucleic acid
gtcgctcttcagcaatacattctt

SEQ ID NO: 66 - DNA, 24 bp, Artificial
  Synthetic nucleic acid
gctgtgaggtcgctcttcagcaat

SEQ ID NO: 67 - DNA, 24 bp, Artificial
  Synthetic nucleic acid
ggctgtgaggtcgctcttcagcaa

SEQ ID NO: 68 - DNA, 24 bp, Artificial
  Synthetic nucleic acid
tcgctcttcagcaatacattctta

SEQ ID NO: 69 - DNA, 24 bp, Artificial
  Synthetic nucleic acid
tgtgaggtcgctcttcagcaatac

SEQ ID NO: 70 - DNA, 24 bp, Artificial
  Synthetic nucleic acid
gaggtcgctcttcagcaatacatt

SEQ ID NO: 71 - RNA, 24 nt, Artificial
  Synthetic nucleic acid
cagcugcaguggcuucaggcccac

SEQ ID NO: 72 - RNA, 24 nt, Artificial
  Synthetic nucleic acid
ugaucuccagcagcugcaguggcu

SEQ ID NO: 73 - RNA, 24 nt, Artificial
  Synthetic nucleic acid
ccccgagccuugaucuccagcagc

SEQ ID NO: 74 - RNA, 24 nt, Artificial
  Synthetic nucleic acid
gucauucaugagcugggccuucca

SEQ ID NO: 75 - RNA, 24 nt, Artificial
  Synthetic nucleic acid
cagcuacaaagucauucaugagcu

SEQ ID NO: 76 - RNA, 24 nt, Artificial
  Synthetic nucleic acid
aagaucuugacagcuacaaaguca

SEQ ID NO: 77 - RNA, 24 nt, Artificial
  Synthetic nucleic acid
cuggagugggaagaucuugacagc

SEQ ID NO: 78 - RNA, 24 nt, Artificial
  Synthetic nucleic acid
aacuguagcagguucucgugcuuc

SEQ ID NO: 79 - RNA, 24 nt, Artificial
  Synthetic nucleic acid
cuugagguaauccgugagggagcc

SEQ ID NO: 80 - RNA, 24 nt, Artificial
  Synthetic nucleic acid
gauguuccccuugagguaauccgu

SEQ ID NO: 81 - RNA, 24 nt, Artificial
  Synthetic nucleic acid
ccaugugaugauguuccccuugag

SEQ ID NO: 82 - RNA, 24 nt, Artificial
  Synthetic nucleic acid
caguucguuccaugugaugauguu

SEQ ID NO: 83 - RNA, 24 nt, Artificial
  Synthetic nucleic acid
uacaugacacaguucguuccaugu

SEQ ID NO: 84 - RNA, 24 nt, Artificial
  Synthetic nucleic acid
cgucucugcuacaugacacaguuc

SEQ ID NO: 85 - RNA, 24 nt, Artificial
  Synthetic nucleic acid
cucgugacaucgucucugcuacau

SEQ ID NO: 86 - RNA, 24 nt, Artificial
  Synthetic nucleic acid
uaugagaggccucgugacaucguc

SEQ ID NO: 87 - RNA, 24 nt, Artificial
  Synthetic nucleic acid
cucaugcagguaugagaggccucg

SEQ ID NO: 88 - RNA, 24 nt, Artificial
  Synthetic nucleic acid
agggcacauccucaugcagguaug

SEQ ID NO: 89 - RNA, 24 nt, Artificial
  Synthetic nucleic acid
ccacggcaccagggcacauccuca

SEQ ID NO: 90 - RNA, 24 nt, Artificial
  Synthetic nucleic acid
gugaggucgcucuucagcaauaca

SEQ ID NO: 91 - RNA, 24 nt, Artificial
  Synthetic nucleic acid
agcacggcugugaggucgcucuuc

SEQ ID NO: 92 - RNA, 24 nt, Artificial
  Synthetic nucleic acid
aagucagccagcacggcugugagg

SEQ ID NO: 93 - RNA, 24 nt, Artificial
  Synthetic nucleic acid
gccaagccaaagucagccagcacg

SEQ ID NO: 94 - RNA, 24 nt, Artificial
  Synthetic nucleic acid
aaucgaacagccaagccaaaguca

SEQ ID NO: 95 - RNA, 24 nt, Artificial
  Synthetic nucleic acid
ccuggcucaaaucgaacagccaag

SEQ ID NO: 96 - RNA, 24 nt, Artificial
  Synthetic nucleic acid
ggagguuucccuggcucaaaucga

SEQ ID NO: 97 - RNA, 24 nt, Artificial
  Synthetic nucleic acid
guguccccuggagguuucccuggc

SEQ ID NO: 98 - RNA, 24 nt, Artificial
  Synthetic nucleic acid
cuguccguggguguccccuggagg

SEQ ID NO: 99 - RNA, 24 nt, Artificial
  Synthetic nucleic acid
agccauguaccgucucgugccuac

SEQ ID NO: 100 - RNA, 24 nt, Artificial
  Synthetic nucleic acid
caccucaggagccauguaccgucu

SEQ ID NO: 101 - RNA, 24 nt, Artificial
  Synthetic nucleic acid
ucucuggaaguugauggcucccuc

SEQ ID NO: 102 - RNA, 24 nt, Artificial
  Synthetic nucleic acid
gaaggcaucucucuggaaguugau

SEQ ID NO: 103 - RNA, 24 nt, Artificial
  Synthetic nucleic acid
caaugcgcaggaaggcaucucucu

SEQ ID NO: 104 - RNA, 24 nt, Artificial
  Synthetic nucleic acid
gcauacaugucaaugcgcaggaag

SEQ ID NO: 105 - RNA, 24 nt, Artificial
  Synthetic nucleic acid
cccacagcaccaaccccauggcau

SEQ ID NO: 106 - RNA, 24 nt, Artificial
  Synthetic nucleic acid
gacacaagcucccacagcaccaac

SEQ ID NO: 107 - RNA, 24 nt, Artificial
  Synthetic nucleic acid
cuugcagcgagacacaagcuccca

SEQ ID NO: 108 - RNA, 24 nt, Artificial
  Synthetic nucleic acid
cgucugcagccuugcagcgagaca

SEQ ID NO: 109 - RNA, 24 nt, Artificial
  Synthetic nucleic acid
gcagcauguacucauccacggguc

SEQ ID NO: 110 - RNA, 24 nt, Artificial
  Synthetic nucleic acid
uccaacgaagggugcuggccaauc

SEQ ID NO: 111 - RNA, 24 nt, Artificial
  Synthetic nucleic acid
cugcagcuccuccaacgaagggug

SEQ ID NO: 112 - protein, 14 aa, Artificial
  Synthetic peptide
KKRTLRKNDRKKRC

(19) United States
(12) Patent Application Publication
(10) Pub. No.: US 2022/0119818 A1
(43) Pub. Date: Apr. 21, 2022

(54) MYOSTATIN SIGNAL INHIBITOR

(71) Applicant: NIPPON SHINYAKU CO., LTD., Kyoto-shi, Kyoto (JP)

(72) Inventor: Shinichiro NAKAGAWA, Ibaraki (JP)

(73) Assignee: NIPPON SHINYAKU CO., LTD., Kyoto-shi, Kyoto (JP)

(21) Appl. No.: 17/417,436

(22) PCT Filed: Dec. 26, 2019
(86) PCT No.: PCT/JP2019/051651

§ 371 Date: Jun. 23, 2021

(30) Foreign Application Priority Data
Dec. 28, 2018 (GB) 1821269.6

(51) Int. Cl. C12N 15/113; A61K 47/54; A61K 47/64; A61K 31/7088; A01K 67/027

(52) U.S. Cl. CPC C12N 15/1138; A61K 47/549; A61K 47/64; A61K 31/7088; A01K 67/0276; A01K 2217/058; C12N 2320/33; C12N 2310/314; C12N 2310/3233; C12N 2310/3513; C12N 2310/11

(57) ABSTRACT

The present invention provides a new approach for inhibiting myostatin signaling by targeting ACVR2B at the mRNA level.

### BACKGROUND ART

Myostatin also known as GDF8 was discovered in 1997 as a novel cytokine belonging to the TGF-β superfamily. The tissue expression of myostatin is specific in skeletal muscle which is the main tissue responsible for motor and metabolic activities. Animals harboring myostatin deficiency mutations show significant muscular hypertrophy where the amount of skeletal muscle increases to twice the size of their wild-type counterpart (McPherron et al., Nature. 387(6628):83-90, 1997). Based on this observation, myostatin is considered to serve as an important factor in controlling skeletal muscular volume.

When myostatin transduces its signal to the interior of a cell, it undergoes a process like other TGF-β where it first binds to a type II receptor, which then associates with a type I receptor to form a ligand-receptor complex. Through this process each of the receptors undergoes phosphorylation on its intercellular domain, which results in the signal transduction through Smad-dependent or Smad-independent pathway (Chang et al., Endocrine Reviews. 23(6):787-823, 2002). Both type I and II receptors are encoded by multiple genes, respectively. Each molecule belonging to the TGF-β superfamily binds to a specific combination of receptors. Myostatin binds to a combination of ALK4 or ALK5 for type I receptor and ACVR2B for type II receptor. However, said combination is not exclusive to myostatin, and used by some other TGF-β superfamily molecules including GDF11, activin A, and so on (Wakefield and Hill. Nat Rev Cancer. 13(5):328-41, 2013, doi:10.1038/nrc3500. Thus, binding of ligands other than myostatin to ACVR2B/ALK4 or ACVR2B/ALK5 may trigger the transduction of suppressive signals to muscle volume as does myostatin binding. Indeed, it has been reported that administering a neutral antibody against ACVR2B or soluble ACVR2B wherein the transmembrane domain and downstream regions thereof are replaced with an antibody-Fc domain, to a mouse deficient of the myostatin gene further increases muscle volume in addition to the increase caused by myostatin deficiency (see Lach-Trifilieff et al., Mol Cell Biol. 34(4):606-18, 2014. doi: 10.1128/MCB.01307-13; Lee et al., Proc Natl Acad Sci USA. 102(50):18117-22, 2005). This further increase suggests there exists additional factors to myostatin which suppress muscle volume by binding to ACVR2B.

Means for reducing myostatin signalling may be useful in the treatment or prevention of particular muscle wasting and other muscle related diseases. The present invention provides a new approach for inhibiting myostatin signaling by targeting ACVR2B at the mRNA level.

### SUMMARY OF THE INVENTION

According to a first aspect of the invention there is provided a compound that is capable of allowing a target cell to produce a mutant activin receptor type-2B (ACVR2B) mRNA where a part of the mRNA sequence that encodes some or all of the intracellular region of wild-type ACVR2B is absent.

According to a variation of the first aspect of the invention there is provided a compound that is capable of causing activin receptor type-2B (ACVR2B) protein to be produced as a truncated version which lacks part of the intracellular region of wild-type ACVR2B, or a pharmaceutically acceptable salt or hydrate thereof.

In particular embodiments, the compound is an oligonucleotide that is capable of effecting exon skipping of one or more of exons, 5, 6, 7, 8, 9 and 10 of ACVR2B.

According to a second aspect of the invention there is provided a pharmaceutical composition comprising the compound or pharmaceutically acceptable salt or hydrate thereof of the first aspect of the invention.

According to a third aspect of the invention there is provided the compound or pharmaceutically acceptable salt or hydrate thereof of the first aspect of the invention or the pharmaceutical composition of the second aspect of the invention for use in therapy. In a particular embodiment the therapy is the prevention or treatment of a muscle wasting disease, a sarcopenic disease or an amyotrophic disease, such as Duchenne muscular dystrophy.

According to a fourth aspect of the invention there is provided a genetically manipulated animal that express a mutant ACVR2B mRNA which lacks part of the intracellular region of ACVR2B.

Particular aspects and embodiments of the invention include:

[1] A compound that is capable of allowing a target cell to produce a mutant activin receptor type-2B (ACVR2B) mRNA where a part of the sequence that encodes some or all of the intracellular region of wild-type ACVR2B is absent, or a pharmaceutically acceptable salt or hydrate thereof.

[2] The compound or pharmaceutically acceptable salt or hydrate thereof according to [1], wherein said intracellular region of wild-type ACVR2B is encoded by exons 5 to 11 of wild-type ACVR2B.

[3] The compound or pharmaceutically acceptable salt or hydrate thereof according to [1] or [2], which is capable of making the target cell produce a truncated ACVR2B protein that lacks part of the intracellular region of wild-type ACVR2B.

[4] The compound or pharmaceutically acceptable salt or hydrate thereof according to [3], wherein the truncated ACVR2B protein lacks all or part of the intracellular region encoded by at least one exon selected from the group consisting of exons 5, 6, 7, 8, 9 and 10 of ACVR2B.

[5] The compound according to any one of [1] to [3], which is an antisense oligomer capable of inducing the skipping of an exon coding for a part of intracellular region of ACVR2B, or a pharmaceutically acceptable salt or hydrate thereof.

[6] The compound or pharmaceutically acceptable salt or hydrate thereof according to [5], wherein said exon to be skipped is selected from the group consisting of exons 5, 6, 7, 8, 9 and 10 of ACVR2B.

[7] The compound according to [5] or [6], which comprises 10-50 nucleobases, or pharmaceutically acceptable salt or hydrate thereof.

[8] The compound according to any one of [5] to [7], comprising a sequence complementary to 10 to 50 consecutive nucleotides of an exon selected from the group consisting of exons 5, 6, 7, 8, 9 and 10 of ACVR2B, or pharmaceutically acceptable salt or hydrate thereof.

[9] The compound or pharmaceutically acceptable salt or hydrate thereof according to any one of [5] to [8], wherein the exon comprises a sequence selected from the group consisting of SEQ ID NOs: 1 to 6.

[10] The compound according to any one of [5] to [9], comprising a nucleotide sequence selected from the group consisting of SEQ ID NOs: 12 to 36 and 43 to 111, or pharmaceutically acceptable salt or hydrate thereof.

[11] The compound according to any one of [5] to [10], consisting of a nucleotide sequence selected from the group consisting of SEQ ID NOs: 12 to 36 and 43 to 111, or pharmaceutically acceptable salt or hydrate thereof.

[12] The compound or pharmaceutically acceptable salt or hydrate thereof according to any one of [5] to [11], wherein the antisense oligomer is an oligonucleotide.

[13] The compound or pharmaceutically acceptable salt or hydrate thereof according to [12], wherein at least one sugar moiety and/or at least one phosphate bond moiety in the oligonucleotide is modified.

[14] The compound or pharmaceutically acceptable salt or hydrate thereof according to [13], wherein the modified sugar moiety is a ribose in which the —OH group at the 2′-position is substituted with any group selected from the group consisting of OR, R, R′OR, SH, SR, NH₂, NHR, NR₂, N₃, CN, F, Cl, Br and I (wherein R represents alkyl or aryl, and R′ represents alkylene).

[15] The compound or pharmaceutically acceptable salt or hydrate thereof according to [13] or [14], wherein the modified phosphate bond moiety is one selected from the group consisting of a phosphorothioate bond, a phosphorodithioate bond, an alkylphosphonate bond, a phosphoroamidate bond and a boranophosphate bond.

[16] The compound or pharmaceutically acceptable salt or hydrate thereof according to any one of [5] to [11], wherein the antisense oligomer comprises at least one morpholino ring.

[17] The compound according to [16], which is a morpholino oligomer or phosphorodiamidate morpholino oligomer, or pharmaceutically acceptable salt or hydrate thereof.

[18] The compound according to [16] or [17], having any one of the groups represented by chemical formulae (1) to (3) shown below at its 5′-terminal end, or pharmaceutically acceptable salt or hydrate thereof.

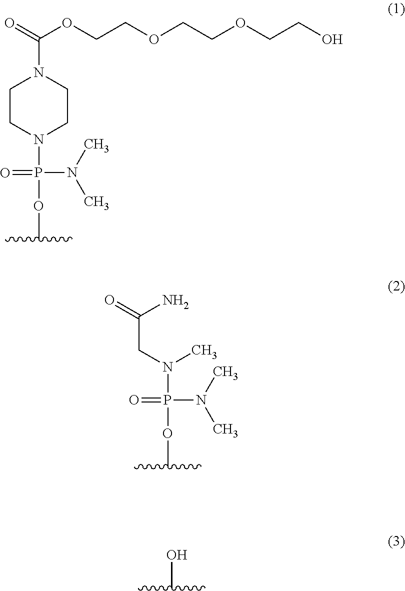

[19] A compound which is a conjugate wherein a cell penetrating peptide is bonded to the compound according to any one of [1] to [18], or pharmaceutically acceptable salt or hydrate thereof.

[20] A pharmaceutical composition comprising the compound or pharmaceutically acceptable salt or hydrate thereof according to any one of [1] to [19].

[21] The pharmaceutical composition according to [20], which further comprises at least one pharmaceutically acceptable carrier or additive.

[22] The pharmaceutical composition according to [20] or [21], which is lyophilized.

[23] The compound or pharmaceutically acceptable salt or hydrate thereof according to any one of [1] to [19] or the pharmaceutical composition according to any one of [20] to [22] for use in therapy in a subject.

[24] The compound or pharmaceutically acceptable salt or hydrate thereof for use or pharmaceutical composition for use according to [23], wherein the therapy is the prevention or treatment of an amyotrophic disease, a muscle wasting disease or a sarcopenic disease in a subject.

[25] The compound or pharmaceutically acceptable salt or hydrate for use or pharmaceutical composition for use according to [24], wherein the amyotrophic disease is Duchenne muscular dystrophy.

[26] The compound or pharmaceutically acceptable salt or hydrate for use or pharmaceutical composition for use according to any one of [23] to [25], wherein the subject is a human.

[27] A method for treating an amyotrophic disease, a muscle wasting disease or a sarcopenic disease in a subject, which comprises administering to said subject a therapeutically effective amount of the compound or pharmaceutically acceptable salt or hydrate thereof according to any one of [1] to [19] or the pharmaceutical composition according to any one of [20] to [22].

[28] The method according to [27], wherein the amyotrophic disease is Duchenne muscular dystrophy.

[29] The method according to [27] or [28], wherein the subject is a human.

[30] Use of the compound or pharmaceutically acceptable salt or hydrate thereof according to any one of [1] to [19] in the manufacture of a medicament for preventing or treating an amyotrophic disease, a muscle wasting disease or a sarcopenic disease in a subject.

[31] The use according to [30], wherein the amyotrophic disease is Duchenne muscular dystrophy.

[32] The use according to [30] or [31], wherein the subject is a human.

[33] A genetically manipulated animal that expresses a mutant activin receptor type-2B (ACVR2B) mRNA where a part of the sequence that encodes some or all of the intracellular region of wild-type ACVR2B is absent.

### BRIEF DESCRIPTION OF FIGURES

FIG. 1 shows the skipping efficiency (%) of each exon by the indicated phosphorodiamidate morpholino oligomers (PMOs) (a and b), phosphorothioate (PS) oligonucleotides (c) or a PMO-peptide conjugate (d).

FIG. 2 shows the suppression of myostatin signaling by the expression of truncated ACVR2B wherein the truncated ACVR2B is expressed dominantly over the endogenous wild-type ACVR2B.

FIG. 3 shows the suppression of SMAD7 mRNA expression which serves as an index of myostatin signal intensity.

### DETAILED DESCRIPTION OF THE INVENTION

The present invention will be described in more detail below. The following embodiments are intended to describe the present invention by way of example only and it is not intended to limit the present invention only to these embodiments. The present invention can be implemented in various modes without departing from the spirit of the present invention. Nucleotide sequences are presented in a manner where 5′ end is placed on the left end and 3′ end is placed on the right end. Amino acid sequences are presented in a manner where N terminus is placed on the left end and C terminus is placed on the right end.

Compound

The present invention provides a compound that is capable of allowing a target cell to produce a mutant activin receptor type-2B (ACVR2B) mRNA where a part of the mRNA sequence that encodes some or all of the intracellular region of wild-type ACVR2B is absent, or a pharmaceutically acceptable salt or hydrate thereof.

ACVR2B protein is also known as ActRIIB and consists of 512 amino acids. The cytogenic map location of ACVR2B is 3p22-p21.3. ACVR2B consists of three main domains, an extracellular ligand binding domain, a transmembrane domain, and an intracellular serine/threonine kinase domain. Ishikawa et al. (Journal of Human Genetics volume 43, pages 132-134 (1998)) reported that the ACVR2B gene contains 11 exons and spans approximately 30 kb. The mRNA sequence of the wild-type human ACVR2B (hereinafter referred to as “wild-type ACVR2B”) is disclosed in NCBI Reference Sequence: NM_001106.4 and herein in SEQ ID NO: 8.

A representative coding sequence (CDS) of human ACVR2B is shown in SEQ ID NO: 10. A nucleotide sequence of ACVR2B CDS is not be limited to one shown as SEQ ID NO: 10, and includes variant sequences having 90% or more, 91% or more, 92% or more, 93% or more, 94% or more, 95% or more, 96% or more, 97% or more, 98% or more, 99% or more, or 100% of the length of SEQ ID NO: 10 and a sequence identity of 90% or more, 91% or more, 92% or more, 93% or more, 94% or more, 95% or more, 96% or more, 97% or more, 98% or more, 99% or more, or 100% to SEQ ID NO: 10.

A representative amino acid sequence of human ACVR2B protein is shown in SEQ ID NO: 11. An amino acid sequence of ACVR2B protein is not be limited to one shown as SEQ ID NO: 11, and includes variant sequences having 90% or more, 91% or more, 92% or more, 93% or more, 94% or more, 95% or more, 96% or more, 97% or more, 98% or more, 99% or more, or 100% of the length of SEQ ID NO: 11 and a sequence identity of 90% or more, 91% or more, 92% or more, 93% or more, 94% or more, 95% or more, 96% or more, 97% or more, 98% or more, 99% or more, or 100% to SEQ ID NO: 11.

When the compound of the present invention is provided to a cell expressing ACVR2B, it causes the cell to produce a mutant ACVR2B mRNA where a part of the mRNA sequence that encodes some or all of the intracellular region of wild-type ACVR2B is absent. A “mutant ACVR2B mRNA where a part of the mRNA sequence that encodes some or all of the intracellular region of wild-type ACVR2B is absent” (hereinafter referred to as “the mutant ACVR2B mRNA of the present invention” means a mutant/variant ACVR2B mRNA which lacks a part of the sequence found in wild-type ACVR2B mRNA, and wherein said sequence which is absent relative to wild-type ACVR2B encodes some or all of the intracellular region of wild-type ACVR2B, or a mutant ACVR2B mRNA which lacks part of the sequence found in wild-type ACVR2B mRNA that encodes some or all of the intracellular region of wild-type ACVR2B.

The intracellular region of human ACVR2B consists of 159^{st} to 512^{nd} amino acids, inclusive, from the N-terminal side. A mRNA sequence that encodes some or all of the intracellular region of a representative wild-type ACVR2B is shown in SEQ ID NO: 9.

The mutant ACVR2B mRNA of the present invention may lack 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064 or 1065 nucleotides of SEQ ID NO:9.

As demonstrated in the Examples section herein, disruption of the intracellular region of human ACVR2B at the mRNA level efficiently reduces myostatin signalling. Thus, the intracellular region of ACVR2B is a good target for disrupting the myostatin signal.

In one embodiment, the compound of the present invention is capable of causing the target cell to produce a truncated ACVR2B protein that lacks part of the intracellular region of wild-type ACVR2B protein, e.g. such as one with the sequence in SEQ ID NO: 11.

As used herein, “truncated ACVR2B protein that lacks part of the intracellular region of wild-type ACVR2B” (hereinafter interchangeably referred to as “truncated version of ACVR2B protein” or “truncated ACVR2B protein”) refers to any truncated version of ACVR2B protein which lacks at least one amino acid in said intracellular region of wild-type ACVR2B. The part of the intracellular region of ACVR2B which is absent relative to wild-type ACVR2B refers to one or more amino acids present in wild-type ACVR2B but not in the truncated ACVR2B. For example, the truncated version of ACVR2B protein may lack 1 amino acid, or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, or 354 amino acids of the intracellular region found in wild-type ACVR2B protein, e.g. such as the one shown as SEQ ID NO: 11. As will be apparent, a truncated version/variant does not merely cover versions that have one or more amino acids removed from the carboxy or amino termini of the protein but also covers variants that lack one or more amino acids from within the ACVR2B protein.

The part of the intracellular region of ACVR2B which is absent or lacking in the truncated version is encoded by all or part of at least one exon selected from the group consisting of exons 5, 6, 7, 8, 9, 10 and 11 of wild-type ACVR2B.

As used herein “capable of causing ACVR2B protein to be produced as a truncated version” means that the compound of the present invention allows a cell to which the compound is added to synthesize or produce a truncated ACVR2B as explained more fully herein.

Because the extracellular and transmembrane regions of ACVR2B are still present, the truncated version of ACVR2B of the invention may still be capable of binding to its native ligands, but in respect of binding of the myostatin ligand, the transduction efficiency of the myostatin signal may be reduced as compared to that of wild-type ACVR2B.

Examples of the native ligands that can bind ACVR2B include activin-A, activin-B, GDF1, GDF3, NODAL, GDF11, myostatin (which is also known as GDF8), BMP2, BMP5, GDF5 (which is also known as BMP14), GDF6, GDF7, BMP5, BMP6, BMP7 and BMP8. A preferred example of the ligand is myostatin which is also known as GDF8.

As used herein “transduction” of a signal is intended to mean relaying the signal by activating downstream factors relevant to the signal, or by inactivating downstream factors relevant to the signal.

In a particular embodiment, the truncated version of ACVR2B protein of the invention is able to bind to myostatin.

The truncated ACVR2B protein of the invention transduces signals upon binding of a ligand to ACVR2B but with less intensity than wild-type ACVR2B. In an embodiment, the truncated ACVR2B protein transduces myostatin signal with less intensity than wild-type ACVR2B upon the binding of myostatin thereto. As used herein, the term “less intensity than wild-type ACVR2B” refers to a reduction in signal intensity (ability to signal) by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% compared to the signal intensity transduced by binding of the same ligand to wild-type ACVR2B. Signal intensity can be determined indirectly by quantitating the mRNA expression level of one or more genes whose expression is triggered by the transduced signal. For example, the signal intensity of myostatin triggered signaling can be gauged by measuring the mRNA expression level of SMAD7. In this case, the compound of the present invention is capable of reducing the intensity of myostatin signal to a level, which correlates in a reduction of mRNA expression level of SMAD7 by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% compared to that caused by myostatin signaling of wild-type ACVR2B.

In another embodiment, the mutant ACVR2B mRNA of the present invention may lack a part of the sequence that encodes some or all of the intracellular region of wild-type ACVR2B because of a frameshift mutation. Such a frameshift mutation may generate a different reading frame downstream therefrom than in the wild-type mRNA. Thus, in this case, the mutant ACVR2B mRNA of the present invention lacks a part of the sequence that encodes some or all of the intracellular region of wild-type ACVR2B.

Such frameshift may also cause the mutant ACVR2B mRNA of the present invention to go through nonsense-mediated mRNA decay (NMD) of ACVR2B. NMD is a mechanism for controlling the quality of mRNAs that all eukaryotic organisms have, and destroys abnormal mRNAs having a stop codon at a position upstream (towards 5′ end) to the original stop codon, typically caused through mutations. When some exons, in particular exons having sequences in the length of non-multiples of three nucleotides (i.e. not 3N where N is a given integer), become skipped, the translation frame of triplets becomes shifted, and this may generate a novel stop codon upstream to the original stop codon. For example, when exon 8 of ACVR2B is skipped, exons 7 and 9 become directly joined. The reading frame at the join of exons 7 and 9, “AG/GTAG” is composed of 2 nucleotides at the most 3′ end of exon 7, i.e. “AG”, and 4 nucleotides at the most 5′ end of exon 9, i.e. “GTAG”. The AGG TAG encodes arginine (Arg) and a stop codon, generating a nonsense mutant-like mRNA. Such a mutant mRNA may then be destroyed by NMD. On the other hand, in wild-type ACVR2B, the reading frame positioned at the joint of exons 7 and 8, “AG/GGAU” is composed of 2 nucleotides at the most 3′ end of exon 7, i.e. “AG”, and 4 nucleotides at the most 5′ end of exon 8, i.e. “GGAU”. The AGG GAU encodes arginine (Arg) and aspartic acid (Asp).

In the situation where a new stop codon is generated due to a frame shift, the mutant ACVR2B mRNA of the present invention is produced, but is then typically degraded/decayed through NMD.

As used herein a “target cell” is a cell to which the compound of the present invention is introduced and may be any cells expressing ACVR2B (e.g. wild-type ACVR2B). Example of the target cell includes myocyte, myoblast, or myotube cell. In an embodiment, the target cell is an animal cell. In another embodiment, the target cell is a mammal cell. In another embodiment, the target cell is a human cell.

The compound of the present invention is any compound that it is capable of causing ACVR2B mRNA and/or protein to be produced as a truncated version which lacks part of the intracellular region of wild-type ACVR2B. Examples of suitable compounds of the present invention include: a CRISPR-CAS9 guide RNA sequence which with the appropriate endonuclease is capable of excising/removing a part of ACVR2B gene encoding a part of the intracellular region thereof, an antisense oligomer for skipping at least one exon encoding a part of the intracellular region ACVR2B, and loxP system compounds for knocking out a part of ACVR2B gene which encodes a part of the intracellular region thereof.

As would be appreciated by a person of skill in the art, other well-known gene editing techniques such as TALENs or Zinc fingers (ZFN) could also be employed to generate a truncated version of ACVR2B of the invention.

When CRISPR-CAS9 is used to inhibit the myostatin signal, a guide RNA having a sequence complementary to a target sequence of genomic DNA which encode ACVR2B or a part of ACVR2B (e.g. the intracellular region of wild-type ACVR2B) is introduced to a target cell, whereby identifying a target sequence to be cleaved. Cas9 protein introduced to the target cell cleaves the double stranded part composed of the genomic DNA and guide RNA. Through a process of repairing the cleavage site, a mutation(s) is caused by deletion and/or insertion of nucleotides, thereby causing the knock-out of all or part of ACVR2B. Examples of the target sequence of genomic DNA includes any sequence of exons, e.g. exons 1 to 11 of ACVR2B. Suitably, the target sequence of genomic DNA includes any sequence of at least one exon selected from the group consisting of exons 5, 6, 7, 8, 9, 10 and 11 or group consisting of exons 5, 6, 7, 8, 9 and 10 of ACVR2B. In one embodiment, the target sequence of genomic DNA includes any sequence of at least one exon selected from the group consisting of exons 5, 6, 7, 9 and 10 of ACVR2B. In another embodiment, the target sequence of genomic DNA includes any sequence of at least one exon selected from the group consisting of exons 5, 6, 9 and 10 of ACVR2B. In another embodiment, the target sequence of genomic DNA includes any sequence of at least one exon selected from the group consisting of exons 5, 6 and 10 of ACVR2B. In another embodiment, the target sequence of genomic DNA includes any sequence of at least one exon selected from the group consisting of exons 5 and 6 of ACVR2B. In another embodiment, the target sequence of genomic DNA includes any sequence of at least one exon selected from the group consisting of exons 7, 8 and 9 of ACVR2B. In another embodiment, the target sequence of genomic DNA includes any sequence of at least one exon selected from the group consisting of exons 7 and 8 of ACVR2B. In another embodiment, the target sequence of genomic DNA is exon 5 of ACVR2B. In another embodiment, the target sequence of genomic DNA is exon 6 of ACVR2B. In another embodiment, the target sequence of genomic DNA is exon 7 of ACVR2B. In another embodiment, the target sequence of genomic DNA is exon 8 of ACVR2B. In another embodiment, the target sequence of genomic DNA is exon 9 of ACVR2B. In another embodiment, the target sequence of genomic DNA is exon 10 of ACVR2B. In another embodiment, the target sequence of genomic DNA is exon 11 of ACVR2B. In another embodiment, the target sequence of genomic DNA includes any sequence of at least one exon selected from the group consisting of exons 7, 8, 9 and 10, or group consisting of exons 5, 6, 7, 8, 9 and 10 of ACVR2B. Alternatively, introns may be targeted by CRISPR-CAS9. For example, introns 7 and 8 which sandwich exon 8 may be cleaved. When the cleaved sites are being repaired, exon 8 may become absent to generate exon 8-deleted mutant mRNA. Similarly, introns 4 and 5, or introns 5 and 6, or introns 6 and 7, or introns 8 and 9, or introns 9 and 10, or introns 10 and 11 may be targeted to cleave. Thus, the compound of the present invention may be a guide RNA for CRISPR-CAS9 as described above, or a DNA (such as an expression plasmid) which provides a guide RNA as its transcript, or CAS9 (or Cas9-like) protein, or a DNA (such as an expression plasmid) which encodes and provides CAS9 (or Cas9-like) protein, or a combination thereof.

When siRNA is used to inhibit the myostatin signal, an siRNA designed to target a sequence of ACVR2B mRNA is introduced to a target cell. When the guide strand of the siRNA thus introduced hybridizes to the targeted sequence, then an endogenous RISC protein in the target cell identifies the double stranded part composed of the guide strand and the targeted mRNA strand, and cleaves the targeted sequence of the mRNA. By doing so, the ACVR2B protein level is reduced. Thus, in another embodiment, the compound of the present invention may be an siRNA or a DNA (such as an expression plasmid) which provides an siRNA as its transcript.

Antisense Oligomer

A mutant ACVR2B mRNA can also be produced intracellularly by contacting the cell with an antisense oligonucleotide (AON) capable of inducing exon skipping of one or more of the ACVR2B exons that encode the intracellular region of the protein to the cell.

In one embodiment, the compound of the present invention is an antisense oligomer capable of inducing the skipping of an exon coding for a part of the intracellular region of ACVR2B (hereinafter referred to as “the antisense oligomer of the present invention” or “the antisense oligomer”). Suitably, the exon to be skipped is one selected from the group consisting of exons 5, 6, 7, 8, 9, 10 and 11 or group consisting of exons 5, 6, 7, 8, 9 and 10 of ACVR2B. In one embodiment, the exon to be skipped is one selected from the group consisting of exons 5, 6, 7, 9 and 10. In another embodiment, the exon to be skipped is exon 5. Yet in another embodiment, the exon to be skipped is exon 6. Yet in another embodiment, the exon to be skipped is exon 7. Yet in another embodiment, the exon to be skipped is exon 8. Yet in another embodiment, the exon to be skipped is exon 9. Yet in another embodiment, the exon to be skipped is exon 10. Yet in another embodiment, the exon to be skipped is exon 11. Yet in another embodiment, the exon to be skipped is one selected from the group consisting of exons 7, 8, 9 and 10, or group consisting of exons 5, 6, 7, 8, 9 and 10 of ACVR2B.

Representative nucleotide sequences of exons 5, 6, 7, 8, 9, 10 and 11 are those shown as SEQ ID NOs: 1, 2, 3, 4, 5, 6 and 7, respectively. Nucleotide sequences of exons 5, 6, 7, 8, 9, 10 and 11 are not be limited to those shown as SEQ ID NOs: 1, 2, 3, 4, 5, 6 and 7, and include variant sequences having 90% or more, 91% or more, 92% or more, 93% or more, 94% or more, 95% or more, 96% or more, 97% or more, 98% or more, 99% or more, or 100% of the length of SEQ ID NOs: 1, 2, 3, 4, 5, 6 and 7 respectively and a sequence identity of 90% or more, 91% or more, 92% or more, 93% or more, 94% or more, 95% or more, 96% or more, 97% or more, 98% or more, 99% or more, or 100% to SEQ ID NOs: 1, 2, 3, 4, 5, 6 and 7 respectively.

The term “capable of inducing the skipping of an exon coding for a part of intracellular region of ACVR2B” means that following binding of the antisense oligomer of the present invention to its target site of an exon coding for a part of the intracellular region of the transcript (e.g., pre-mRNA) of the ACVR2B gene (e.g., human ACVR2B gene), said exon is spliced out. For example, if the antisense oligomer of the present invention binds to a part of exon 6 of ACVR2B pre-mRNA, the nucleotide sequence corresponding to the 5′ end of the exon downstream to exon 6, i.e. exon 7, is spliced at the 3′ side of the nucleotide sequence corresponding to the 3′ end of the exon upstream to exon 6, i.e. exon 5. This is caused by a disruption of the normal splicing mechanism following binding of the antisense oligomer of the present invention. The ACVR2B polypeptide encoded by the mRNA would then include amino acids encoded by exon 5 joined to exon 7, with those encoded by exon 6 being omitted (absent) from the truncated ACVR2B variant.

Herein, the term “binding” means that when the antisense oligomer of the present invention is brought into contact with (e.g. mixed with) copies of transcript of ACVR2B gene (e.g. human ACVR2B gene), the complementary sequences hybridize under physiological conditions to form a double stranded nucleic acid. The term “under physiological conditions” refers to conditions that mimic the in vivo environment in terms of pH, salt composition and temperature. Suitable conditions can be any combination of the following temperature, pH and salt concentration:

### Temperature: 25° C. to 40° C., 35° C. to 38° C., 36° C. to 38° C., or 37° C.;

pH: pH 5 to 8, pH 6 to 8, pH 7 to 8, or pH 7.4; and
Salt concentration: 100 to 200 mM, 130 to 170 mM, 140 to 160 mM, or 150 mM of sodium chloride concentration.

As used herein, “sequence identity” and “homology” with respect to a nucleotide sequence refers to the percentage of nucleotide residues in a candidate target sequence that are identical with the nucleotide residues in a subject nucleotide sequence, after aligning the sequences and allowing for gaps, if necessary, to achieve the maximum percent sequence identity, and not considering any conservative substitutions as part of the sequence identity. Alignment for purposes of determining percent nucleotide sequence identity can be achieved in various ways that are within the skill of one in the art, for instance, using publicly available computer software such as BLAST, BLAST-2, ClustalW2, ALIGN or MEGALIGN™ (DNASTAR) software. Those skilled in the art can determine appropriate parameters for measuring alignment, including any algorithms needed to achieve maximal alignment over the full length of the sequences being compared. For example, sequence identity of two or more nucleotide sequences can be determined by using the algorithm of Karlin and Altschul, BLAST (Basic Local Alignment Search Tool) (Proc. Natl. Acad. Sci. USA 872264-2268, 1990; Proc Natl Acad Sci USA 90: 5873, 1993). Based on the algorithm of BLAST, programs called BLASTN and BLASTX have been developed (Altschul S F, et al: J Mol Biol 215: 403, 1990). If BLASTN is used for nucleotide sequence analysis, parameters may be set to, for example, score=100 and wordlength=12. If BLAST and Gapped BLAST programs are used, default parameters in each program may be used.

The antisense oligomer of the present invention is an oligonucleotide or modified oligonucleotide. As used herein, an “oligonucleotide” is a sequence of linked nucleotides of a length as defined below that may or may not include modifications. A modified oligonucleotide is described in detail elsewhere.

The antisense oligomer of the present invention may have a length of 10 to 70 nucleotides, such as: 11 to 70, 12 to 70, 13 to 70, 14 to 70, 15 to 70, 16 to 70, 17 to 70, 18 to 70, 19 to 70, 20 to 70, 21 to 70, 22 to 70, 23 to 70, 24 to 70, 25 to 70, 10 to 65, 11 to 65, 12 to 65, 13 to 65, 14 to 65, 15 to 65, 16 to 65, 17 to 65, 18 to 65, 19 to 65, 20 to 65, 21 to 65, 22 to 65, 23 to 65, 24 to 65, 25 to 65, 10 to 60, 11 to 60, 12 to 60, 13 to 60, 14 to 60, 15 to 60, 16 to 60, 17 to 60, 18 to 60, 19 to 60, 20 to 60, 21 to 60, 22 to 60, 23 to 60, 24 to 60, 25 to 60, 10 to 55, 11 to 55, 12 to 55, 13 to 55, 14 to 55, 15 to 55, 16 to 55, 17 to 55, 18 to 55, 19 to 55, 20 to 55, 21 to 55, 22 to 55, 23 to 55, 24 to 55, 25 to 55, 10 to 50, 11 to 50, 12 to 50, 13 to 50, 14 to 50, 15 to 50, 16 to 50, 17 to 50, 18 to 50, 19 to 50, 20 to 50, 21 to 50, 22 to 50, 23 to 50, 24 to 50, 25 to 50, 10 to 45, 11 to 45, 12 to 45, 13 to 45, 14 to 45, 15 to 45, 16 to 45, 17 to 45, 18 to 45, 19 to 45, 20 to 45, 21 to 45, 22 to 45, 23 to 45, 24 to 45, 25 to 45, 10 to 40, 11 to 40, 12 to 40, 13 to 40, 14 to 40, 15 to 40, 16 to 40, 17 to 40, 18 to 40, 19 to 40, 20 to 40, 21 to 40, 22 to 40, 23 to 40, 24 to 40, 25 to 40, 10 to 38, 11 to 38, 12 to 38, 13 to 38, 14 to 38, 15 to 38, 16 to 38, 17 to 38, 18 to 38, 19 to 38, 20 to 38, 21 to 38, 22 to 38, 23 to 38, 24 to 38, 25 to 38, 10 to 36, 11 to 36, 12 to 36, 13 to 36, 14 to 36, 15 to 36, 16 to 36, 17 to 36, 18 to 36, 19 to 36, 20 to 36, 21 to 36, 22 to 36, 23 to 36, 24 to 36, 25 to 36, 10 to 35, 11 to 35, 12 to 35, 13 to 35, 14 to 35, 15 to 35, 16 to 35, 17 to 35, 18 to 35, 19 to 35, 20 to 35, 21 to 35, 22 to 35, 23 to 35, 24 to 35, 25 to 35, 10 to 34, 11 to 34, 12 to 34, 13 to 34, 14 to 34, 15 to 34, 16 to 34, 17 to 34, 18 to 34, 19 to 34, 20 to 34, 21 to 34, 22 to 34, 23 to 34, 24 to 34, 25 to 34, 10 to 33, 11 to 33, 12 to 33, 13 to 33, 14 to 33, 15 to 33, 16 to 33, 17 to 33, 18 to 33, 19 to 33, 20 to 33, 21 to 33, 22 to 33, 23 to 33, 24 to 33, 25 to 33, 10 to 32, 11 to 32, 12 to 32, 13 to 32, 14 to 32, 15 to 32, 16 to 32, 17 to 32, 18 to 32, 19 to 32, 20 to 32, 21 to 32, 22 to 32, 23 to 32, 24 to 32, 25 to 32, 10 to 30, 11 to 30, 12 to 30, 13 to 30, 14 to 30, 15 to 30, 16 to 30, 17 to 30, 18 to 30, 19 to 30, 20 to 30, 21 to 30, 22 to 30, 23 to 30, 24 to 30, 25 to 30, 10 to 29, 11 to 29, 12 to 29, 13 to 29, 14 to 29, 15 to 29, 16 to 29, 17 to 29, 18 to 29, 19 to 29, 20 to 29, 21 to 29, 22 to 29, 23 to 29, 24 to 29, 25 to 29, 10 to 28, 11 to 28, 12 to 28, 13 to 28, 14 to 28, 15 to 28, 16 to 28, 17 to 28, 18 to 28, 19 to 28, 20 to 28, 21 to 28, 22 to 28, 23 to 28, 24 to 28, 25 to 28, 10 to 27, 11 to 27, 12 to 27, 13 to 27, 14 to 27, 15 to 27, 16 to 27, 17 to 27, 18 to 27, 19 to 27, 20 to 27, 21 to 27, 22 to 27, 23 to 27, 24 to 27, 25 to 27, 10 to 26, 11 to 26, 12 to 26, 13 to 26, 14 to 26, 15 to 26, 16 to 26, 17 to 26, 18 to 26, 19 to 26, 20 to 26, 21 to 26, 22 to 26, 23 to 26, 24 to 26, 25 to 26, 10 to 25, 11 to 25, 12 to 25, 13 to 25, 14 to 25, 15 to 25, 16 to 25, 17 to 25, 18 to 25, 19 to 25, 20 to 25, 21 to 25, 22 to 25, 23 to 25, 24 to 25, 10 to 24, 11 to 24, 12 to 24, 13 to 24, 14 to 24, 15 to 24, 16 to 24, 17 to 24, 18 to 24, 19 to 24, 20 to 24, 21 to 24, 22 to 24, 23 to 24, 10 to 23, 11 to 23, 12 to 23, 13 to 23, 14 to 23, 15 to 23, 16 to 23, 17 to 23, 18 to 23, 19 to 23, 20 to 23, 21 to 23, 22 to 23, 10 to 22, 11 to 22, 12 to 22, 13 to 22, 14 to 22, 15 to 22, 16 to 22, 17 to 22, 18 to 22, 19 to 22, 20 to 22, 21 to 22, 10 to 21, 11 to 21, 12 to 21, 13 to 21, 14 to 21, 15 to 21, 16 to 21, 17 to 21, 18 to 21, 19 to 21, 20 to 21, 10 to 20, 11 to 20, 12 to 20, 13 to 20, 14 to 20, 15 to 20, 16 to 20, 17 to 20, 18 to 20, 19 to 20, 10 to 19, 11 to 19, 12 to 19, 13 to 19, 14 to 19, 15 to 19, 16 to 19, 17 to 19, 18 to 19, 10 to 18, 11 to 18, 12 to 18, 13 to 18, 14 to 18, 15 to 18, 16 to 18, 17 to 18, 10 to 17, 11 to 17, 12 to 17, 13 to 17, 14 to 17, 15 to 17, 16 to 17, 10 to 16, 11 to 16, 12 to 16, 13 to 16, 14 to 16, 15 to 16, 10 to 15, 11 to 15, 12 to 15, 13 to 15 and 14 to 15 nucleotides from its 5′ end to the 3′ end (hereinafter referred to as “exemplary length range of the antisense oligomer of the present invention”). The antisense oligomer of the present invention may have a length of 10, 11, 12, 13, 14, 15, 16, 17, 18, 19, 20, 21, 22, 23, 24, 25, 26, 27, 28, 29, 30, 33, 32, 33, 34, 35, 36, 37, 38, 39, 40, 44, 42, 43, 44, 45, 46, 47, 48, 49, 50, 55, 52, 53, 54, 55, 56, 57, 58, 59, 60, 66, 62, 63, 64, 65, 66, 67, 68, 69, or 70 nucleotides from its 5′ end to the 3′ end (hereinafter referred to as “exemplary length of the antisense oligomer of the present invention”). Particularly suitable ranges for the length of the oligomer of the invention include: 15 to 45, 17 to 35, 15 to 24, 15 to 26, and 20 to 40 nucleotides from its 5′ end to the 3′ end.

The antisense oligomer of the present invention comprises a nucleotide sequence complementary to a part of the nucleotide sequence of an exon selected from the group consisting of exons 5, 6, 7, 8, 9, 10 and 11 of ACVR2B or the group consisting of exons 5, 6, 7, 8, 9 and 10 of ACVR2B. The part of the nucleotide sequence of an exon selected from the group consisting of exons 5, 6, 7, 8, 9, 10 and 11 of ACVR2B or the group consisting of exons 5, 6, 7, 8, 9 and 10 of ACVR2B is herein also referred to as “the target sequence”. Representative nucleotide sequences of exons 5, 6, 7, 8, 9, 10 and 11 are those shown as SEQ ID NOs: 1, 2, 3, 4, 5, 6 and 7, respectively. Nucleotide sequences of exons 5, 6, 7, 8, 9, 10 and 11 are not be limited to those shown as SEQ ID NOs: 1, 2, 3, 4, 5, 6 and 7, and include variant sequences having 90% or more, 91% or more, 92% or more, 93% or more, 94% or more, 95% or more, 96% or more, 97% or more, 98% or more, 99% or more, or 100% sequence identity to a sequence disclosed in any of SEQ ID NOs: 1, 2, 3, 4, 5, 6 and 7 respectively. Thus, according to particular embodiments of the invention, the oligomer of the invention comprises a nucleotide sequence which is complementary to a sequence with 90% or more, 91% or more, 92% or more, 93% or more, 94% or more, 95% or more, 96% or more, 97% or more, 98% or more, 99% or more, or 100% sequence identity to a sequence disclosed in any of SEQ ID NOs: 1, 2, 3, 4, 5, 6 and 7 respectively.

The target sequence may be of any length as long as it is the same or shorter than the length of the antisense oligomer of the present invention. For example, the target sequence may be in the length of 10 to 70 nucleotides, such as: 11 to 70, 12 to 70, 13 to 70, 14 to 70, 15 to 70, 16 to 70, 17 to 70, 18 to 70, 19 to 70, 20 to 70, 21 to 70, 22 to 70, 23 to 70, 24 to 70, 25 to 70, 10 to 65, 11 to 65, 12 to 65, 13 to 65, 14 to 65, 15 to 65, 16 to 65, 17 to 65, 18 to 65, 19 to 65, 20 to 65, 21 to 65, 22 to 65, 23 to 65, 24 to 65, 25 to 65, 10 to 60, 11 to 60, 12 to 60, 13 to 60, 14 to 60, 15 to 60, 16 to 60, 17 to 60, 18 to 60, 19 to 60, 20 to 60, 21 to 60, 22 to 60, 23 to 60, 24 to 60, 25 to 60, 10 to 55, 11 to 55, 12 to 55, 13 to 55, 14 to 55, 15 to 55, 16 to 55, 17 to 55, 18 to 55, 19 to 55, 20 to 55, 21 to 55, 22 to 55, 23 to 55, 24 to 55, 25 to 55, 10 to 50, 11 to 50, 12 to 50, 13 to 50, 14 to 50, 15 to 50, 16 to 50, 17 to 50, 18 to 50, 19 to 50, 20 to 50, 21 to 50, 22 to 50, 23 to 50, 24 to 50, 25 to 50, 10 to 45, 11 to 45, 12 to 45, 13 to 45, 14 to 45, 15 to 45, 16 to 45, 17 to 45, 18 to 45, 19 to 45, 20 to 45, 21 to 45, 22 to 45, 23 to 45, 24 to 45, 25 to 45, 10 to 40, 11 to 40, 12 to 40, 13 to 40, 14 to 40, 15 to 40, 16 to 40, 17 to 40, 18 to 40, 19 to 40, 20 to 40, 21 to 40, 22 to 40, 23 to 40, 24 to 40, 25 to 40, 10 to 38, 11 to 38, 12 to 38, 13 to 38, 14 to 38, 15 to 38, 16 to 38, 17 to 38, 18 to 38, 19 to 38, 20 to 38, 21 to 38, 22 to 38, 23 to 38, 24 to 38, 25 to 38, 10 to 36, 11 to 36, 12 to 36, 13 to 36, 14 to 36, 15 to 36, 16 to 36, 17 to 36, 18 to 36, 19 to 36, 20 to 36, 21 to 36, 22 to 36, 23 to 36, 24 to 36, 25 to 36, 10 to 35, 11 to 35, 12 to 35, 13 to 35, 14 to 35, 15 to 35, 16 to 35, 17 to 35, 18 to 35, 19 to 35, 20 to 35, 21 to 35, 22 to 35, 23 to 35, 24 to 35, 25 to 35, 10 to 34, 11 to 34, 12 to 34, 13 to 34, 14 to 34, 15 to 34, 16 to 34, 17 to 34, 18 to 34, 19 to 34, 20 to 34, 21 to 34, 22 to 34, 23 to 34, 24 to 34, 25 to 34, 10 to 33, 11 to 33, 12 to 33, 13 to 33, 14 to 33, 15 to 33, 16 to 33, 17 to 33, 18 to 33, 19 to 33, 20 to 33, 21 to 33, 22 to 33, 23 to 33, 24 to 33, 25 to 33, 10 to 32, 11 to 32, 12 to 32, 13 to 32, 14 to 32, 15 to 32, 16 to 32, 17 to 32, 18 to 32, 19 to 32, 20 to 32, 21 to 32, 22 to 32, 23 to 32, 24 to 32, 25 to 32, 10 to 30, 11 to 30, 12 to 30, 13 to 30, 14 to 30, 15 to 30, 16 to 30, 17 to 30, 18 to 30, 19 to 30, 20 to 30, 21 to 30, 22 to 30, 23 to 30, 24 to 30, 25 to 30, 10 to 29, 11 to 29, 12 to 29, 13 to 29, 14 to 29, 15 to 29, 16 to 29, 17 to 29, 18 to 29, 19 to 29, 20 to 29, 21 to 29, 22 to 29, 23 to 29, 24 to 29, 25 to 29, 10 to 28, 11 to 28, 12 to 28, 13 to 28, 14 to 28, 15 to 28, 16 to 28, 17 to 28, 18 to 28, 19 to 28, 20 to 28, 21 to 28, 22 to 28, 23 to 28, 24 to 28, 25 to 28, 10 to 27, 11 to 27, 12 to 27, 13 to 27, 14 to 27, 15 to 27, 16 to 27, 17 to 27, 18 to 27, 19 to 27, 20 to 27, 21 to 27, 22 to 27, 23 to 27, 24 to 27, 25 to 27, 10 to 26, 11 to 26, 12 to 26, 13 to 26, 14 to 26, 15 to 26, 16 to 26, 17 to 26, 18 to 26, 19 to 26, 20 to 26, 21 to 26, 22 to 26, 23 to 26, 24 to 26, 25 to 26, 10 to 25, 11 to 25, 12 to 25, 13 to 25, 14 to 25, 15 to 25, 16 to 25, 17 to 25, 18 to 25, 19 to 25, 20 to 25, 21 to 25, 22 to 25, 23 to 25, 24 to 25, 10 to 24, 11 to 24, 12 to 24, 13 to 24, 14 to 24, 15 to 24, 16 to 24, 17 to 24, 18 to 24, 19 to 24, 20 to 24, 21 to 24, 22 to 24, 23 to 24, 10 to 23, 11 to 23, 12 to 23, 13 to 23, 14 to 23, 15 to 23, 16 to 23, 17 to 23, 18 to 23, 19 to 23, 20 to 23, 21 to 23, 22 to 23, 10 to 22, 11 to 22, 12 to 22, 13 to 22, 14 to 22, 15 to 22, 16 to 22, 17 to 22, 18 to 22, 19 to 22, 20 to 22, 21 to 22, 10 to 21, 11 to 21, 12 to 21, 13 to 21, 14 to 21, 15 to 21, 16 to 21, 17 to 21, 18 to 21, 19 to 21, 20 to 21, 10 to 20, 11 to 20, 12 to 20, 13 to 20, 14 to 20, 15 to 20, 16 to 20, 17 to 20, 18 to 20, 19 to 20, 10 to 19, 11 to 19, 12 to 19, 13 to 19, 14 to 19, 15 to 19, 16 to 19, 17 to 19, 18 to 19, 10 to 18, 11 to 18, 12 to 18, 13 to 18, 14 to 18, 15 to 18, 16 to 18, 17 to 18, 10 to 17, 11 to 17, 12 to 17, 13 to 17, 14 to 17, 15 to 17, 16 to 17, 10 to 16, 11 to 16, 12 to 16, 13 to 16, 14 to 16, 15 to 16, 10 to 15, 11 to 15, 12 to 15, 13 to 15 and 14 to 15 nucleotides from the 5′ end to the 3′ end. The target sequence may have a length of 10, 11, 12, 13, 14, 15, 16, 17, 18, 19, 20, 21, 22, 23, 24, 25, 26, 27, 28, 29, 30, 33, 32, 33, 34, 35, 36, 37, 38, 39, 40, 44, 42, 43, 44, 45, 46, 47, 48, 49, 50, 55, 52, 53, 54, 55, 56, 57, 58, 59, 60, 66, 62, 63, 64, 65, 66, 67, 68, 69, or 70 nucleotides from its 5′ end to the 3′ end.

The nucleotide sequence complementary to the target sequence (hereinafter referred to as “hybridizing sequence”) should be the same length as the target sequence. Thus, exemplary length of the hybridizing sequence includes: 10 to 70 nucleotides, such as 11 to 70, 12 to 70, 13 to 70, 14 to 70, 15 to 70, 16 to 70, 17 to 70, 18 to 70, 19 to 70, 20 to 70, 21 to 70, 22 to 70, 23 to 70, 24 to 70, 25 to 70, 10 to 65, 11 to 65, 12 to 65, 13 to 65, 14 to 65, 15 to 65, 16 to 65, 17 to 65, 18 to 65, 19 to 65, 20 to 65, 21 to 65, 22 to 65, 23 to 65, 24 to 65, 25 to 65, 10 to 60, 11 to 60, 12 to 60, 13 to 60, 14 to 60, 15 to 60, 16 to 60, 17 to 60, 18 to 60, 19 to 60, 20 to 60, 21 to 60, 22 to 60, 23 to 60, 24 to 60, 25 to 60, 10 to 55, 11 to 55, 12 to 55, 13 to 55, 14 to 55, 15 to 55, 16 to 55, 17 to 55, 18 to 55, 19 to 55, 20 to 55, 21 to 55, 22 to 55, 23 to 55, 24 to 55, 25 to 55, 10 to 50, 11 to 50, 12 to 50, 13 to 50, 14 to 50, 15 to 50, 16 to 50, 17 to 50, 18 to 50, 19 to 50, 20 to 50, 21 to 50, 22 to 50, 23 to 50, 24 to 50, 25 to 50, 10 to 45, 11 to 45, 12 to 45, 13 to 45, 14 to 45, 15 to 45, 16 to 45, 17 to 45, 18 to 45, 19 to 45, 20 to 45, 21 to 45, 22 to 45, 23 to 45, 24 to 45, 25 to 45, 10 to 40, 11 to 40, 12 to 40, 13 to 40, 14 to 40, 15 to 40, 16 to 40, 17 to 40, 18 to 40, 19 to 40, 20 to 40, 21 to 40, 22 to 40, 23 to 40, 24 to 40, 25 to 40, 10 to 38, 11 to 38, 12 to 38, 13 to 38, 14 to 38, 15 to 38, 16 to 38, 17 to 38, 18 to 38, 19 to 38, 20 to 38, 21 to 38, 22 to 38, 23 to 38, 24 to 38, 25 to 38, 10 to 36, 11 to 36, 12 to 36, 13 to 36, 14 to 36, 15 to 36, 16 to 36, 17 to 36, 18 to 36, 19 to 36, 20 to 36, 21 to 36, 22 to 36, 23 to 36, 24 to 36, 25 to 36, 10 to 35, 11 to 35, 12 to 35, 13 to 35, 14 to 35, 15 to 35, 16 to 35, 17 to 35, 18 to 35, 19 to 35, 20 to 35, 21 to 35, 22 to 35, 23 to 35, 24 to 35, 25 to 35, 10 to 34, 11 to 34, 12 to 34, 13 to 34, 14 to 34, 15 to 34, 16 to 34, 17 to 34, 18 to 34, 19 to 34, 20 to 34, 21 to 34, 22 to 34, 23 to 34, 24 to 34, 25 to 34, 10 to 33, 11 to 33, 12 to 33, 13 to 33, 14 to 33, 15 to 33, 16 to 33, 17 to 33, 18 to 33, 19 to 33, 20 to 33, 21 to 33, 22 to 33, 23 to 33, 24 to 33, 25 to 33, 10 to 32, 11 to 32, 12 to 32, 13 to 32, 14 to 32, 15 to 32, 16 to 32, 17 to 32, 18 to 32, 19 to 32, 20 to 32, 21 to 32, 22 to 32, 23 to 32, 24 to 32, 25 to 32, 10 to 30, 11 to 30, 12 to 30, 13 to 30, 14 to 30, 15 to 30, 16 to 30, 17 to 30, 18 to 30, 19 to 30, 20 to 30, 21 to 30, 22 to 30, 23 to 30, 24 to 30, 25 to 30, 10 to 29, 11 to 29, 12 to 29, 13 to 29, 14 to 29, 15 to 29, 16 to 29, 17 to 29, 18 to 29, 19 to 29, 20 to 29, 21 to 29, 22 to 29, 23 to 29, 24 to 29, 25 to 29, 10 to 28, 11 to 28, 12 to 28, 13 to 28, 14 to 28, 15 to 28, 16 to 28, 17 to 28, 18 to 28, 19 to 28, 20 to 28, 21 to 28, 22 to 28, 23 to 28, 24 to 28, 25 to 28, 10 to 27, 11 to 27, 12 to 27, 13 to 27, 14 to 27, 15 to 27, 16 to 27, 17 to 27, 18 to 27, 19 to 27, 20 to 27, 21 to 27, 22 to 27, 23 to 27, 24 to 27, 25 to 27, 10 to 26, 11 to 26, 12 to 26, 13 to 26, 14 to 26, 15 to 26, 16 to 26, 17 to 26, 18 to 26, 19 to 26, 20 to 26, 21 to 26, 22 to 26, 23 to 26, 24 to 26, 25 to 26, 10 to 25, 11 to 25, 12 to 25, 13 to 25, 14 to 25, 15 to 25, 16 to 25, 17 to 25, 18 to 25, 19 to 25, 20 to 25, 21 to 25, 22 to 25, 23 to 25, 24 to 25, 10 to 24, 11 to 24, 12 to 24, 13 to 24, 14 to 24, 15 to 24, 16 to 24, 17 to 24, 18 to 24, 19 to 24, 20 to 24, 21 to 24, 22 to 24, 23 to 24, 10 to 23, 11 to 23, 12 to 23, 13 to 23, 14 to 23, 15 to 23, 16 to 23, 17 to 23, 18 to 23, 19 to 23, 20 to 23, 21 to 23, 22 to 23, 10 to 22, 11 to 22, 12 to 22, 13 to 22, 14 to 22, 15 to 22, 16 to 22, 17 to 22, 18 to 22, 19 to 22, 20 to 22, 21 to 22, 10 to 21, 11 to 21, 12 to 21, 13 to 21, 14 to 21, 15 to 21, 16 to 21, 17 to 21, 18 to 21, 19 to 21, 20 to 21, 10 to 20, 11 to 20, 12 to 20, 13 to 20, 14 to 20, 15 to 20, 16 to 20, 17 to 20, 18 to 20, 19 to 20, 10 to 19, 11 to 19, 12 to 19, 13 to 19, 14 to 19, 15 to 19, 16 to 19, 17 to 19, 18 to 19, 10 to 18, 11 to 18, 12 to 18, 13 to 18, 14 to 18, 15 to 18, 16 to 18, 17 to 18, 10 to 17, 11 to 17, 12 to 17, 13 to 17, 14 to 17, 15 to 17, 16 to 17, 10 to 16, 11 to 16, 12 to 16, 13 to 16, 14 to 16, 15 to 16, 10 to 15, 11 to 15, 12 to 15, 13 to 15 and 14 to 15 nucleotides from the 5′ end to the 3′ end. The hybridizing sequence may have a length of 10, 11, 12, 13, 14, 15, 16, 17, 18, 19, 20, 21, 22, 23, 24, 25, 26, 27, 28, 29, 30, 33, 32, 33, 34, 35, 36, 37, 38, 39, 40, 44, 42, 43, 44, 45, 46, 47, 48, 49, 50, 55, 52, 53, 54, 55, 56, 57, 58, 59, 60, 66, 62, 63, 64, 65, 66, 67, 68, 69, or 70 nucleotides from its 5′ end to the 3′ end.

Examples of suitable hybridizing sequence include any of SEQ ID NOs: 12 to 36 and 43 to 111. The antisense oligomer of the present invention may comprise a nucleotide sequence selected from the group consisting of any of SEQ ID NOs: 12 to 36 and 43 to 111 (hereinafter referred to as “a suitable hybridizing sequence” or “suitable hybridizing sequences”).

The antisense oligomer of the present invention may not necessarily comprise just the hybridizing sequence. As long as the antisense oligomer of the present invention retains skipping activity for at least one exon selected from the group consisting of exons 5, 6, 7, 8, 9, 10 and 11 of ACVR2B or group consisting of exons 5, 6, 7, 8, 9, and 10 of ACVR2B, the antisense oligomer of the present invention may comprise a partial sequence of the above suitable hybridizing sequences. In some embodiment, the antisense oligomer of the present invention may comprise 10, 11, 12, 13, 14, 15, 16, 17, 18, 19, 20, 21, 22, 23, 24 consecutive nucleotides of any one of the sequences disclosed in SEQ ID NOs: 12 to 36 and 43 to 111. In another embodiment, the antisense oligomer of the present invention may comprise at least 10, 11, 12, 13, 14, 15, 16, 17, 18, 19, 20, 21, 22, 23, 24 consecutive nucleotides of any one of the sequences disclosed in SEQ ID NOs: 12 to 36 and 43 to 111.

The antisense oligomer of the present invention may not necessarily comprise just the hybridizing sequence. As long as the antisense oligomer of the present invention retains skipping activity for at least one exon selected from the group consisting of exons 5, 6, 7, 8, 9, 10 and 11 of ACVR2B or group consisting of exons 5, 6, 7, 8, 9, and 10 of ACVR2B, the antisense oligomer of the present invention may comprise additional sequence (bases) that do not complement the target region.

Examples of the sequence targeted by an antisense oligomer of the present invention includes any sequence of exons, e.g. exons 1 to 11 of ACVR2B pre-mRNA. Suitably, the target sequence includes any sequence of at least one exon selected from the group consisting of exons 5, 6, 7, 8, 9, 10 and 11 or group consisting of exons 5, 6, 7, 8, 9 and 10 of ACVR2B pre-mRNA. In one embodiment, the target sequence of ACVR2B pre-mRNA includes any sequence of at least one exon selected from the group consisting of exons 5, 6, 7, 9 and 10. In another embodiment, the target sequence of ACVR2B pre-mRNA includes any sequence of at least one exon selected from the group consisting of exons 5, 6, 9 and 10. In another embodiment, the target sequence of ACVR2B pre-mRNA includes any sequence of at least one exon selected from the group consisting of exons 5, 6 and 10. In another embodiment, the target sequence of ACVR2B pre-mRNA includes any sequence of at least one exon selected from the group consisting of exons 5 and 6. In another embodiment, the target sequence of ACVR2B pre-mRNA includes any sequence of at least one exon selected from the group consisting of exons 7, 8 and 9. In another embodiment, the target sequence of ACVR2B pre-mRNA includes any sequence of at least one exon selected from the group consisting of exons 7 and 8. In another embodiment, the target sequence of ACVR2B pre-mRNA is exon 5. In another embodiment, the target sequence of ACVR2B pre-mRNA is exon 6. In another embodiment, the target sequence of ACVR2B pre-mRNA is exon 7. In another embodiment, the target sequence of ACVR2B pre-mRNA is exon 8. In another embodiment, the target sequence of ACVR2B pre-mRNA is exon 9. In another embodiment, the target sequence of ACVR2B pre-mRNA is exon 10. In another embodiment, the target sequence of ACVR2B pre-mRNA is exon 11. In another embodiment, the target sequence of ACVR2B pre-mRNA includes any sequence of at least one exon selected from the group consisting of exons 7, 8, 9 and 10, or group consisting of exons 5, 6, 7, 8, 9 and 10.

In a particular embodiment the oligomer of the invention has a hybridizing sequence that is the same length as the oligomer or shorter than the oligomer by 1, 2, 3, 4, 5, 6, 7, 8, 9 or 10 nucleotides. For example, the oligomer may be 24 nucleotides in length and have a hybridizing sequence that is the same length, i.e. all 24 nucleotides in the oligomer complement the target region, or it might have a hybridizing sequence that is 20 nucleotides in length (4 shorter than the oligomer length) and the oligomer thus also possesses 4 nucleotides that do not complement the target region (and are thus not part of the hybridizing sequence). Such an oligomer might, for example, have two nucleotides either side of the hybridizing sequence that do not complement the target region, or 3 on one side and one on the other, or 4 at one end.

In addition to the hybridizing sequence or partial hybridizing sequence, the antisense oligomer of the present invention may comprise additional sequences which may or may not contribute to hybridizing to the target sequence. Such additional sequences may be attached to the 5′ end, 3′ end or both ends of the hybridizing sequence or partial hybridizing sequence. In this case the total length of the antisense oligomer of the present invention falls within the exemplary length range of the antisense oligomer of the present invention or the exemplary length of the antisense oligomer of the present invention.

In another embodiment, the antisense oligomer of the present invention may consist of a nucleotide sequence as shown in any of SEQ ID NOs: 12 to 36 and 43 to 111. In some embodiment, the antisense oligomer of the present invention may consist of 5, 6, 7, 8, 9, 10, 11, 12, 13, 14, 15, 16, 17, 18, 19, 20, 21, 22, 23, 24 consecutive nucleotides of a sequence shown in any of SEQ ID NOs: 12 to 36 and 43 to 111.

In another embodiment, the present invention provides a conjugate wherein a functional peptide, e.g. cell penetrating peptide (CPP), is bonded to the antisense oligomer of the present invention. Publicly known functional peptide or commercially available functional peptide can be used in the present invention. The functional peptide that can be used in the present invention include, for example, the arginine-rich peptides disclosed in WO2008/036127; or the peptides targeting organs disclosed in WO2009/005793, such as RXR, RBR and the like; or the peptides comprising an amino acid subunit disclosed in WO2012/150960. The cell penetrating peptides (CPPs) represent short peptide sequences of 10 to about 30 amino acids which can cross the plasma membrane of mammalian cells and may thus improve cellular drug delivery (See, for example, Hum Mol Genet. Aug. 15, 2011; 20(16): 3151-3160; Pharmacology & Therapeutics 154 (2015) 78-86). Publicly known CPPs or commercially available CPPs can be used in the present invention. The CPPs that can be used in the present invention include, for example; the CPPs listed in Table 1 on page 80 of Pharmacology & Therapeutics 154 (2015) 78-86, such as TAT (48-60), penetratin, polyarginine, Oct4, WT1-pTj, DPV3, Transportan, MAP, VP22, Rep1, KW, KFGF, FGF12, Intefrin β3 peptide, C105Y, TP2; the CPPs listed in paragraph [0085], Table 1 of JP-A-2017-500856 (WO2015/089487), such as DPV10/6, DPV15b, YM-3, Tat, LR11, C45D18, Lyp-1, Lyp-2, BMV GAG, hLF1-22, C45D18, LR20; and the like. CPPs are commercially available from, for example, Funakoshi, Co., Ltd. The commercially available CPPs such as TAT (Funakoshi, Co., Ltd.), penetratin (Funakoshi, Co., Ltd.) and the like, or the publicly known CPPs such as R8 and the like can be used in the present invention. Preferred CPPs which can be used in the present invention include, for example, hLIMK, TAT, penetratin, R8 and the like (see WO2016/187425, WO2018/118662, WO2018/118599, WO2018/118627, EBioMedicine 45 (2019) 630-645 etc.). The CPP can be directly bonded to the antisense oligomer of the present invention, or can be bonded through a linker which can bind a CPP to an antisense oligomer. Publicly known linkers can be used in the present invention. Such linkers include, for example, those described in JP-A-2017-500856 (WO2015/089487), WO2015/089487, WO2009/073809, WO2013/075035, WO2015/105083, WO2014/179620, WO2015/006740, WO2017/010575, and the like. Preferred linkers which can be used in the present invention include, for example, 4-maleimidobutyric acid, a linker that can attach to the functional peptide or the antisense oligomer of the present invention via disulfide bond, and the like. The conjugates of the present invention may be prepared by a method publicly known to a person having an ordinary skill in the art.

Skipping Efficiency:

Whether or not a particular oligomer can effect skipping of an exon or exons in the ACVR2B gene can be assessed or confirmed by introducing the antisense oligomer of the present invention into a cell expressing ACVR2B (e.g., a human rhabdomyosarcoma cell), amplifying the region of ACVR2B mRNA coding for the intracellular region of ACVR2B from the total RNA of the ACVR2B expression cell by RT-PCR or sequence analysis on the PCR amplified product.

The efficiency of skipping may be determined as follows: The RT-PCR reaction solution is measured for the polynucleotide level “A” in the PCR amplified product with exon skipping (e.g. the mRNA amount of truncated ACVR2B) and the polynucleotide level “B” in the PCR amplified product without exon skipping (e.g. the mRNA amount of full-length ACVR2B), followed by calculation based on these measured values of “A” and “B” according to the following equation.

$$Skipping efficiency ( \% ) = \{ A / ( A + B ) \} ⨯ 100$$

In a preferred embodiment, the antisense oligomer of the present invention causes exon skipping with an efficiency of 10% or more, 15% or more, 20% or more, 25% or more, 30% or more, 35% or more, 40% or more, 45% or more, 50% or more, 55% or more, 60% or more, 62.5% or more, 65% or more, 67.5% or more, 70% or more, 72.5% or more, 75% or more, 77.5% or more, 80% or more, 82.5% or more, 85% or more, 87.5% or more, 90% or more, 92.5% or more, 95% or more, 97.5% or more, 98% or more or 99% or more.

Once an effective antisense oligomer is identified, a skilled person may seek to identify a more optimal sequence by designing a variety of antisense oligomers having sequences which overlap with the sequence of the effective antisense oligomer, and testing them using procedures as described herein.

Oligonucleotide, Morpholino Oligomer or Peptide Nucleic Acid Oligomer:

The antisense oligomer of the present invention may be an oligonucleotide, a morpholino oligomer or a peptide nucleic acid (PNA) oligomer, each being in the exemplary length range of the antisense oligomer of the present invention or the exemplary length of the antisense oligomer of the present invention.

The above oligonucleotide (hereinafter referred to as “the oligonucleotide of the present invention”) is an antisense oligomer of the present invention, whose constituent unit is a nucleotide, and such a nucleotide may be any of a ribonucleotide, a deoxyribonucleotide or a modified nucleotide.

The antisense oligonucleotide is typically single stranded.

A modified nucleotide refers to a ribonucleotide or deoxyribonucleotide whose nucleobase, sugar moiety and phosphate bond moiety are all or partly modified.

In the present invention, examples of a nucleobase include adenine, guanine, hypoxanthine, cytosine, thymine, uracil, or modified bases thereof. Such modified bases may be exemplified by pseudouracil, 3-methyluracil, dihydrouracil, 5-alkylcytosines (e.g., 5-methylcytosine), 5-alkyluracils (e.g., 5-ethyluracil), 5-halouracils (e.g., 5-bromouracil), 6-azapyrimidine, 6-alkylpyrimidines (e.g., 6-methyluracil), 2-thiouracil, 4-thiouracil, 4-acetylcytosine, 5-(carboxyhydroxymethyl)uracil, 5-carboxymethylaminomethyl-2-thiouracil, 5-carboxymethylaminomethyluracil, 1-methyladenine, 1-methylhypoxanthine, 2,2-dimethylguanine, 3-methylcytosine, 2-methyladenine, 2-methylguanine, N6-methyladenine, 7-methylguanine, 5-methoxyaminomethyl-2-thiouracil, 5-methylaminomethyluracil, 5-methylcarbonylmethyluracil, 5-methyloxyuracil, 5-methyl-2-thiouracil, 2-methylthio-N6-isopentenyladenine, uracil-5-oxyacetic acid, 2-thiocytosine, purine, 2,6-diaminopurine, 2-aminopurine, isoguanine, indole, imidazole, xanthine and so on, but are not limited thereto.

Modifications to the sugar moiety may be exemplified by modifications at the 2′-position of ribose and modifications at the other positions of sugar. Examples of modifications at the 2′-position of ribose include modifications intended to replace the —OH group at the 2′-position of ribose with OR, R, R′OR, SH, SR, NH₂, NHR, NR₂, N₃, CN, F, Cl, Br or I, wherein R represents alkyl or aryl, and R′ represents alkylene.

Examples of modifications at the other positions of sugar include replacement of 0 with S at the 4′-position of ribose or deoxyribose, and bridging between 2′- and 4′-positions of sugar, as exemplified by LNAs (locked nucleic acids) or ENAs (2′-0,4′-C-ethylene-bridged nucleic acids), but are not limited thereto.

Modifications to the phosphate bond moiety may be exemplified by modifications intended to replace the phosphodiester bond with a phosphorothioate bond, a phosphorodithioate bond, an alkylphosphonate bond, a phosphoroamidate bond or a boranophosphate bond (Enya et al: Bioorganic & Medicinal Chemistry, 2008, 18, 9154-9160) (see, e.g., JP WO2006/129594 and JP WO2006/038608).

In the present invention, alkyl is preferably a linear or branched alkyl containing 1 to 6 carbon atoms. More specifically, examples include methyl, ethyl, n-propyl, isopropyl, n-butyl, isobutyl, sec-butyl, tert-butyl, n-pentyl, isopentyl, neopentyl, tert-pentyl, n-hexyl and isohexyl. Such an alkyl may be substituted with 1 to 3 substituents including halogen, alkoxy, cyano, nitro, etc.

In the present invention, cycloalkyl is preferably a cycloalkyl containing 5 to 12 carbon atoms. More specifically, examples include cyclopentyl, cyclohexyl, cycloheptyl, cyclooctyl, cyclodecyl and cyclododecyl.

In the present invention, halogens include fluorine, chlorine, bromine and iodine.

Alkoxy may be a linear or branched alkoxy containing 1 to 6 carbon atoms, as exemplified by methoxy, ethoxy, n-propoxy, isopropoxy, n-butoxy, isobutoxy, sec-butoxy, tert-butoxy, n-pentyloxy, isopentyloxy, n-hexyloxy, isohexyloxy and so on. Particularly preferred is an alkoxy containing 1 to 3 carbon atoms.

In the present invention, aryl is preferably an aryl containing 6 to 10 carbon atoms. More specifically, examples include phenyl, α-naphthyl and β-naphthyl. Particularly preferred is phenyl. Such an aryl may be substituted with 1 to 3 substituents including alkyl, halogen, alkoxy, cyano, nitro, etc.

In the present invention, alkylene is preferably a linear or branched alkylene containing 1 to 6 carbon atoms. More specifically, examples include methylene, ethylene, trimethylene, tetramethylene, pentamethylene, hexamethylene, 2-(ethyl)trimethylene and 1-(methyl)tetramethylene.

In the present invention, acyl may be a linear or branched alkanoyl or an aroyl. Examples of such an alkanoyl include formyl, acetyl, 2-methylacetyl, 2,2-dimethylacetyl, propionyl, butyryl, isobutyryl, pentanoyl, 2,2-dimethylpropionyl, hexanoyl and so on. Examples of an aroyl include benzoyl, toluoyl and naphthoyl. Such an aroyl may be substituted at any substitutable position and may be substituted with alkyl(s).

The oligonucleotide of the present invention is preferably an antisense oligomer according to the present invention, whose constituent unit is a group represented by the following general formula, in which the —OH group at the 2′-position of ribose is substituted with methoxy and the phosphate bond moiety is a phosphorothioate bond:

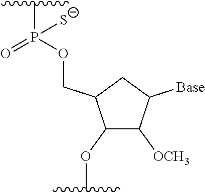

(wherein Base represents a nucleobase).

The oligonucleotide of the present invention may be readily synthesized with various automatic synthesizers (e.g., FOCUS (Aapptec), AKTA oligopilot plus 10/100 (GE Healthcare)), or alternatively, its synthesis may be entrusted to a third party (e.g., Promega, Takara, or Japan Bio Services), etc.

The morpholino oligomer of the present invention is an antisense oligomer according to the present invention, whose constituent unit is a group represented by the following general formula:

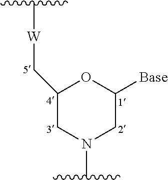

(wherein Base is the same as defined above; and W represents a group represented by any of the following formulae:

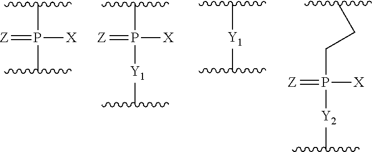

(wherein X represents —CH₂R¹, —O—CH₂R¹, —S—CH₂R¹, —NR²R³ or F;

R¹ represents H or alkyl;

R² and R³, which may be the same or different, each represent H, alkyl, cycloalkyl or aryl;

Y₁ represents 0, S, CH₂ or NR¹;

Y₂ represents 0, S or NR¹; and

Z represents 0 or S)).

The morpholino oligomer is preferably an oligomer whose constituent unit is a group represented by the following formula (i.e., a phosphorodiamidate morpholino oligomer (hereinafter referred to as “PMO”)):

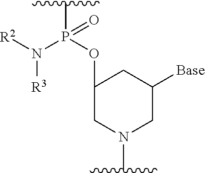

(wherein Base, R² and R³ are the same as defined above).

For example, the morpholino oligomer may be prepared in accordance with WO1991/009033 or WO2009/064471. In particular, PMO may be prepared in accordance with the procedures described in WO2009/064471 or may be prepared in accordance with the procedures shown below.

[Process for PMO Preparation]

As one embodiment of a PMO, a compound represented by the following general formula (I) (hereinafter referred to as PMO (I)) may be given by way of example:

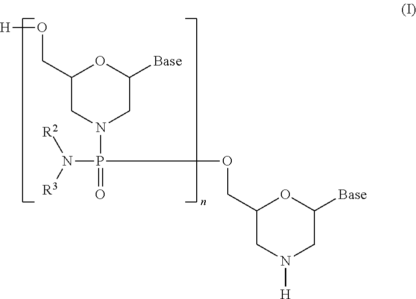

[wherein each Base, R² and R³ are the same as defined above; and n is any integer in the range of 1 to 99, suitably any integer in the range of 13 to 29, 14 to 28 or 15 to 27, 16 to 26, 17 to 25].

PMO (I) may be prepared in accordance with known procedures, for example, by conducting the operations shown in the following steps.

Compounds and reagents used in the following steps are not limited in any way as long as they are commonly used for PMO preparation. Moreover, all the following steps may be accomplished by the liquid phase method or the solid phase method (in accordance with instruction manuals or using a commercially available solid phase automatic synthesizer). When PMO is prepared by the solid phase method, it is desirable to use an automatic synthesizer in terms of simple operation and accurate synthesis.

(1) Step A:

This is a step where a compound represented by the following general formula (II) (hereinafter referred to as compound (II)) is treated with an acid to prepare a compound represented by the following general formula (III) (hereinafter referred to as compound (III)):

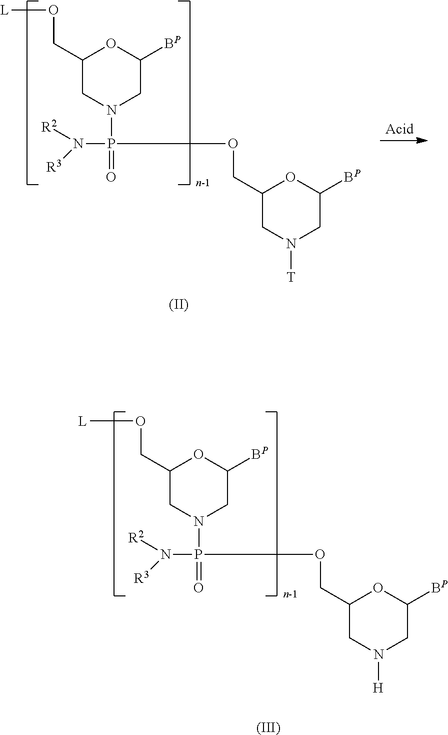

[wherein n, R² and R³ are the same as defined above;

each B^{P} independently represents a nucleobase which may be protected;

T represents a trityl group, a monomethoxytrityl group or a dimethoxytrityl group; and

L represents hydrogen, acyl or a group represented by the following general formula (IV) (hereinafter referred to as group (IV))]:

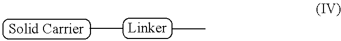

“Nucleobases” possible for B^{P} may be exemplified by the same “nucleobases” as listed for Base, provided that amino groups or hydroxyl groups in these nucleobases for B^{P} may be protected.

Protecting groups for these amino groups are not limited in any way as long as they are used as protecting groups for nucleic acids. More specifically, examples include benzoyl, 4-methoxybenzoyl, acetyl, propionyl, butyryl, isobutyryl, phenylacetyl, phenoxyacetyl, 4-tert-butylphenoxyacetyl, 4-isopropylphenoxyacetyl, and (dimethylamino)methylene.

Protecting groups for hydroxyl groups include, for example, 2-cyanoethyl, 4-nitrophenethyl, phenylsulfonylethyl, methylsulfonylethyl, trimethylsilylethyl, phenyl which may be substituted with 1 to 5 electron withdrawing groups at any substitutable position(s), diphenylcarbamoyl, dimethylcarbamoyl, diethylcarbamoyl, methylphenylcarbamoyl, 1-pyrrolidinylcarbamoyl, morpholinocarbamoyl, 4-(tert-butylcarboxy)benzyl, 4-[(dimethylamino)carboxy]benzyl, and 4-(phenylcarboxy)benzyl (see, e.g., WO2009/064471).

The “solid carrier” is not limited in any way as long as it is a carrier available for use in the solid phase reaction of nucleic acids, but it is desirable to use, for example, a carrier which (i) is sparingly soluble in reagents available for use in the synthesis of morpholino nucleic acid derivatives (e.g., dichloromethane, acetonitrile, tetrazole, N-methylimidazole, pyridine, acetic anhydride, lutidine, trifluoroacetic acid), (ii) is chemically stable against the reagents available for use in the synthesis of morpholino nucleic acid derivatives, (iii) can be chemically modified, (iv) can be loaded with desired morpholino nucleic acid derivatives, (v) has strength sufficient to withstand high pressure during processing, and (vi) has a certain range of particle size and distribution. More specifically, examples include swelling polystyrenes (e.g., aminomethyl polystyrene resin crosslinked with 1% divinylbenzene (200 to 400 mesh) (2.4 to 3.0 mmol/g) (Tokyo Chemical Industry Co., Ltd., Japan), Aminomethylated Polystyrene Resin HCl [divinylbenzene 1%, 100 to 200 mesh] (Peptide Institute, Inc., Japan)), non-swelling polystyrenes (e.g., Primer Support (GE Healthcare)), PEG chain-liked polystyrenes (e.g., NH₂-PEG resin (Watanabe Chemical Industries, Ltd., Japan), TentaGel resin), controlled pore glass (CPG) (e.g., a product of CPG Inc.), oxalylated controlled pore glass (see, e.g., Alul et al., Nucleic Acids Research, Vol. 19, 1527 (1991)), TentaGel support-aminopolyethylene glycol-derivatized support (see, e.g., Wright et al., Tetrahedron Letters, Vol. 34, 3373 (1993)), and a Poros-polystyrene/divinylbenzene copolymer.

As a “linker,” it is possible to use a known linker which is commonly used to link a nucleic acid or a morpholino nucleic acid derivative. Suitable examples include 3-aminopropyl, succinyl, 2,2′-diethanol sulfonyl, and a long-chain alkylamino (LCAA).

Examples of an “acid” available for use in this step include trifluoroacetic acid, dichloroacetic acid or trichloroacetic acid. The amount of an acid to be used is, for example, in the range of 0.1 molar equivalents to 1000 molar equivalents, such as in the range of 1 molar equivalent to 100 molar equivalents, relative to 1 mole of compound (II).

Moreover, it is possible to use an organic amine together with the above acid. Any organic amine may be used for this purpose, and examples include triethylamine. The amount of an organic amine to be used is, for example, reasonably in the range of 0.01 molar equivalents to 10 molar equivalents, such as in the range of 0.1 molar equivalents to 2 molar equivalents, relative to 1 mole of the acid.

In a case where an acid and an organic amine are used as a salt or mixture in this step, examples include a salt or mixture of trifluoroacetic acid and triethylamine, more specifically a mixture containing 2 equivalents of trifluoroacetic acid and 1 equivalent of triethylamine.

An acid available for use in this step may be used by being diluted with an appropriate solvent to give a concentration in the range of 0.1% to 30%. Any solvent may be used for this purpose as long as it is inert to the reaction, and examples include dichloromethane, acetonitrile, alcohols (e.g., ethanol, isopropanol, trifluoroethanol), water, or mixtures thereof.

The reaction temperature in the above reaction is, for example, in the range of 10° C. to 50° C., such as in the range of 20° C. to 40° C. or in the range of 25° C. to 35° C.

The reaction time will vary depending on the type of acid to be used and/or the reaction temperature, but it is generally in the range of 0.1 minutes to 24 hours, and suitably in the range of 1 minute to 5 hours.

Moreover, after completion of this step, a base may optionally be added to neutralize the acid remaining in the system. Any “base” may be used for this purpose and examples include diisopropylethylamine. Such a base may be used by being diluted with an appropriate solvent to give a concentration in the range of 0.1% (v/v) to 30% (v/v).

Any solvent may be used in this step as long as it is inert to the reaction, and examples include dichloromethane, acetonitrile, alcohols (e.g., ethanol, isopropanol, trifluoroethanol), water, or mixtures thereof. The reaction temperature is, for example, in the range of 10° C. to 50° C., such as in the range of 20° C. to 40° C., and suitably in the range of 25° C. to 35° C.

The reaction time will vary depending on the type of base to be used and/or the reaction temperature, but it is generally in the range of 0.1 minutes to 24 hours, and suitably in the range of 1 minute to 5 hours.

It should be noted that compound (II) in which n=1 and L is group (IV), i.e., a compound represented by the following general formula (IIa) (hereinafter referred to as compound (IIa)) may be prepared in accordance with the following procedures:

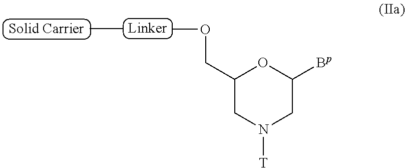

[wherein B^{P}, T, Linker and Solid carrier are the same as defined above].

Step 1:

This is a step where a compound represented by the following general formula (V) is treated with an acylating agent to prepare a compound represented by the following general formula (VI) (hereinafter referred to as compound (VI)):

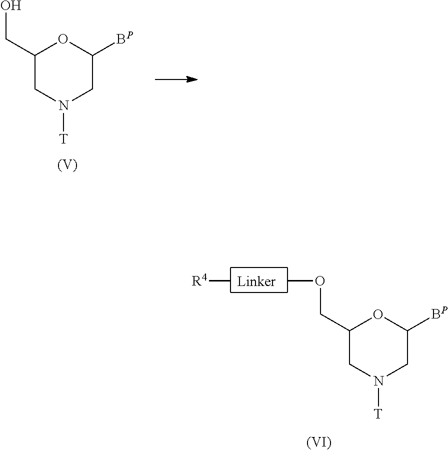

[wherein B^{P}, T and Linker are the same as defined above; and

R⁴ represents a hydroxyl group, halogen, a carboxyl group or amino].

This step may be accomplished starting from compound (V) by any known reaction for linker introduction.

In particular, a compound represented by the following general formula (VIa) may be prepared by any process known as esterification reaction with the use of compound (V) and succinic anhydride:

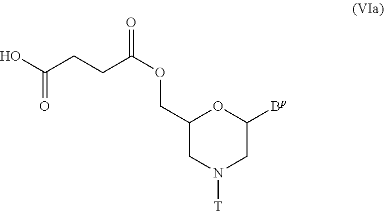

[wherein B^{P} and T are the same as defined above].

Step 2:

This is a step where compound (VI) is reacted with a solid carrier by being treated with a condensing agent or the like to prepare compound (Ha):

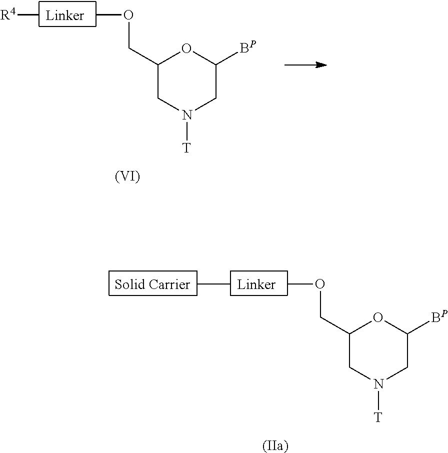

[wherein B^{P}, R⁴, T, Linker and Solid carrier are the same as defined above].

This step may be accomplished by any process known as condensation reaction with the use of compound (VI) and a solid carrier.

Compound (II) in which n=2 to 99 (suitably any integer in the range of 13 to 29, 14 to 28, 15 to 27, 16 to 26, or 17 to 25) and L is group (IV), i.e., a compound represented by the following general formula (IIa2) may be prepared starting from compound (IIa) by repeating desired times Steps A and B of the process for PMO preparation disclosed herein:

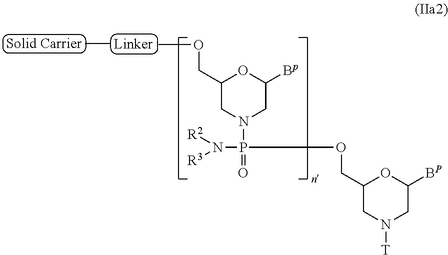

[wherein B^{P}, R², R³, T, Linker and Solid carrier are the same as defined above; and

n′ represents 1 to 98 (in particular embodiments, n′ represents 1 to 28, 1 to 27, 1 to 26, 1 to 25, or 1 to 24)].

(2) Step B:

This is a step where compound (III) is treated with a morpholino monomer compound in the presence of a base to prepare a compound represented by the following general formula (VII) (hereinafter referred to as compound (VII)):

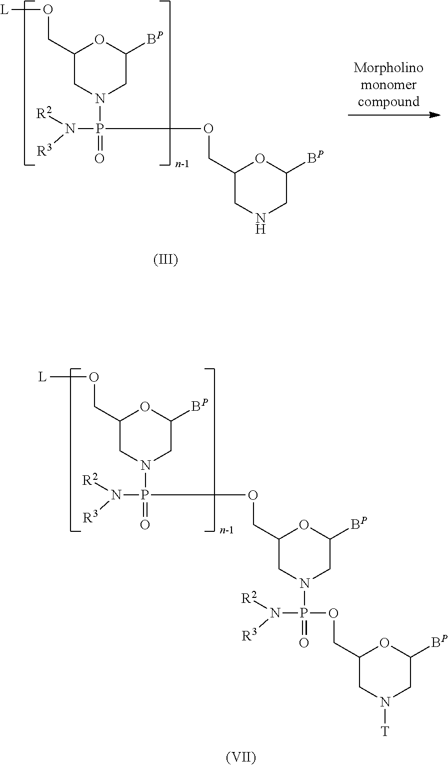

[wherein each B^{P}, L, n, R², R³ and T are the same as defined above].

This step may be accomplished by treating compound (III) with a morpholino monomer compound in the presence of a base.

Such a morpholino monomer compound may be exemplified by a compound represented by the following general formula (VIII):

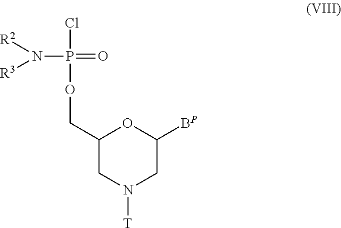

[wherein B^{P}, R², R³ and T are the same as defined above].

Examples of a “base” available for use in this step include diisopropylethylamine, triethylamine or N-ethylmorpholine. The amount of a base to be used is, for example, in the range of 1 molar equivalent to 1000 molar equivalents, suitably in the range of 10 molar equivalents to 100 molar equivalents, relative to 1 mole of compound (III).

Such a morpholino monomer compound and a base available for use in this step may be used by being diluted with an appropriate solvent to give a concentration of 0.1% to 30%. Any solvent may be used for this purpose as long as it is inert to the reaction, and examples include N,N-dimethylimidazolidinone, N-methylpiperidone, DMF, dichloromethane, acetonitrile, tetrahydrofuran, or mixtures thereof.

The reaction temperature is, for example, in the range of 0° C. to 100° C., and suitably in the range of 10° C. to 50° C.

The reaction time will vary depending on the type of base to be used and/or the reaction temperature, but it is generally in the range of 1 minute to 48 hours, and suitably in the range of 30 minutes to 24 hours.

Moreover, after completion of this step, an acylating agent may optionally be added. Examples of an “acylating agent” include acetic anhydride, acetic acid chloride and phenoxyacetic anhydride. Such an acylating agent may be used by being diluted with an appropriate solvent to give a concentration in the range of 0.1% to 30%, by way of example. Any solvent may be used for this purpose as long as it is inert to the reaction, and examples include dichloromethane, acetonitrile, tetrahydrofuran, alcohols (e.g., ethanol, isopropanol, trifluoroethanol), water, or mixtures thereof.

If necessary, it is possible to use a base (e.g., pyridine, lutidine, collidine, triethylamine, diisopropylethylamine, N-ethylmorpholine) together with an acylating agent. The amount of an acylating agent to be used is suitably in the range of 0.1 molar equivalents to 10000 molar equivalents, and more suitably in the range of 1 molar equivalent to 1000 molar equivalents. The amount of a base to be used is, for example, in the range of 0.1 molar equivalents to 100 molar equivalents, suitably in the range of 1 molar equivalent to 10 molar equivalents, relative to 1 mole of an acylating agent.

The reaction temperature in this reaction is suitably in the range of 10° C. to 50° C., such as in the range of 20° C. to 40° C., and suitably in the range of 25° C. to 35° C. The reaction time will vary, e.g., depending on the type of acylating agent to be used and/or the reaction temperature, but it is generally in the range of 0.1 minutes to 24 hours, and suitably in the range of 1 minute to 5 hours.

(3) Step C:

This is a step where a deprotecting agent is used to remove the protecting groups from compound (VII) prepared in Step B, thereby preparing a compound represented by general formula (IX):

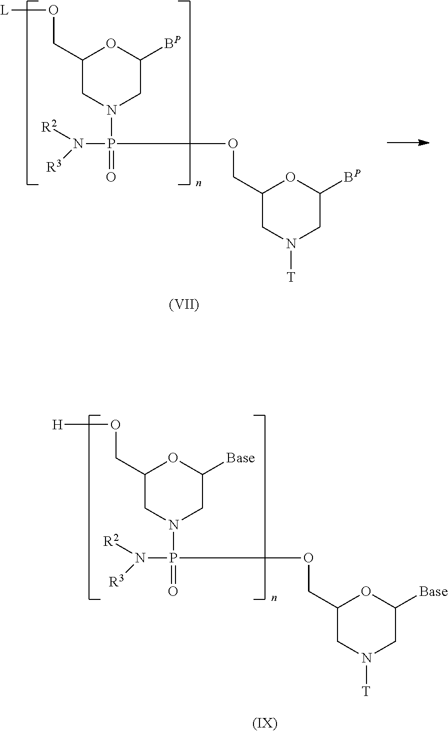

[wherein Base, B^{P}, L, n, R², R³ and T are the same as defined above].

This step may be accomplished by treating compound (VII) with a deprotecting agent.

Examples of a “deprotecting agent” include concentrated aqueous ammonia and methylamine. Such a “deprotecting agent” available for use in this step may be used by being diluted with water, methanol, ethanol, isopropyl alcohol, acetonitrile, tetrahydrofuran, DMF, N,N-dimethylimidazolidinone, N-methylpiperidone, or a mixed solvent thereof. Among them, preferred is ethanol. The amount of a deprotecting agent to be used is, for example, in the range of 1 molar equivalent to 100000 molar equivalents, suitably in the range of 10 molar equivalents to 1000 molar equivalents, relative to 1 mole of compound (VII), by way of example.

The reaction temperature is, for example, in the range of 15° C. to 75° C., suitably in the range of 40° C. to 60° C. or in the range of 50° C. to 60° C. The reaction time for deprotection will vary depending on the type of compound (VII) and/or the reaction temperature, etc., but it is reasonably in the range of 10 minutes to 30 hours, suitably in the range of 30 minutes to 24 hours, and more suitably in the range of 5 hours to 20 hours.

(4) Step D:

This is a step where compound (IX) prepared in Step C is treated with an acid to prepare PMO (I):

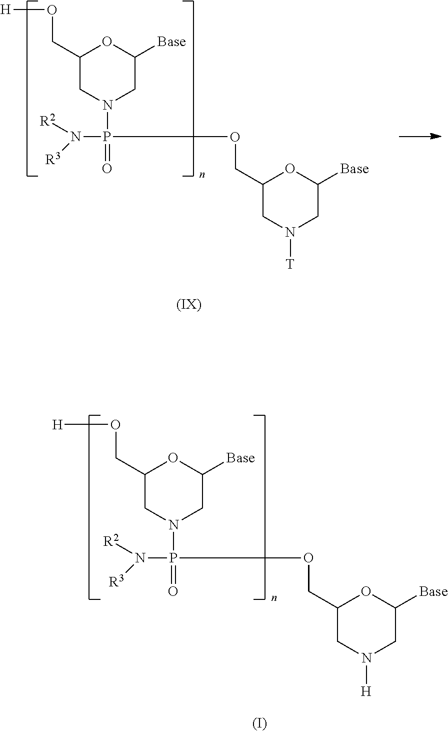

[wherein Base, n, R², R³ and T are the same as defined above].

This step may be accomplished by adding an acid to compound (IX).

Examples of an “acid” available for use in this step include trichloroacetic acid, dichloroacetic acid, acetic acid, phosphoric acid and hydrochloric acid, etc. As to the amount of an acid to be used, it is reasonable to use the acid in an amount to give a solution pH, for example, in the range of 0.1 to 4.0, suitably in the range of 1.0 to 3.0. Any solvent may be used in this step as long as it is inert to the reaction, and examples include acetonitrile, water, or mixed solvents thereof.

The reaction temperature is suitably in the range of 10° C. to 50° C., such as in the range of 20° C. to 40° C. or in the range of 25° C. to 35° C. The reaction time for deprotection will vary depending on the type of compound (IX) and/or the reaction temperature, etc., but it is suitably in the range of 0.1 minutes to 5 hours, such as in the range of 1 minute to 1 hour, and more suitably in the range of 1 minute to 30 minutes.

PMO (I) may be obtained from the reaction mixture obtained in this step by commonly used separation and purification means including extraction, concentration, neutralization, filtration, centrifugation, recrystallization, C₈ to C₁₈ reversed-phase column chromatography, cation exchange column chromatography, anion exchange column chromatography, gel filtration column chromatography, high performance liquid chromatography, dialysis, ultrafiltration and other means, which may be used either alone or in combination, whereby desired PMO (I) can be isolated and purified (see, e.g., WO1991/09033).

In the case of using reversed-phase chromatography for purification of PMO (I), a mixed solution of 20 mM triethylamine/acetate buffer and acetonitrile may be used as an elution solvent, by way of example.

Likewise, in the case of using ion exchange chromatography for purification of PMO (I), a mixed solution of 1 M aqueous sodium chloride and 10 mM aqueous sodium hydroxide may be used, by way of example.

The peptide nucleic acid oligomer is an antisense oligomer according to the present invention, whose constituent unit is a group represented by the following general formula:

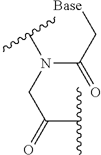

(wherein Base is the same as defined above).

Peptide nucleic acids may be prepared, for example, in accordance with the documents listed below.
1) P. E. Nielsen, M. Egholm, R. H. Berg, O. Buchardt, Science, 254, 1497 (1991)
2) M. Egholm, O. Buchardt, P. E. Nielsen, R. H. Berg, Jacs., 114, 1895 (1992)
3) K. L. Dueholm, M. Egholm, C. Behrens, L. Christensen, H. F. Hansen, T. Vulpius, K. H. Petersen, R. H. Berg, P. E. Nielsen, O. Buchardt, J. Org. Chem., 59, 5767 (1994)
4) L. Christensen, R. Fitzpatrick, B. Gildea, K. H. Petersen, H. F. Hansen, T. Koch, M. Egholm, O. Buchardt, P. E. Nielsen, J. Coull, R. H. Berg, J. Pept. Sci., 1, 175 (1995)
5) T. Koch, H. F. Hansen, P. Andersen, T. Larsen, H. G. Batz, K. Otteson, H. Orum, J. Pept. Res., 49, 80 (1997)

The antisense oligomer of the present invention may be configured such that its 5′-terminal end is any one of the groups represented by chemical formulae (1) to (3) shown below, with (3) —OH being preferred.

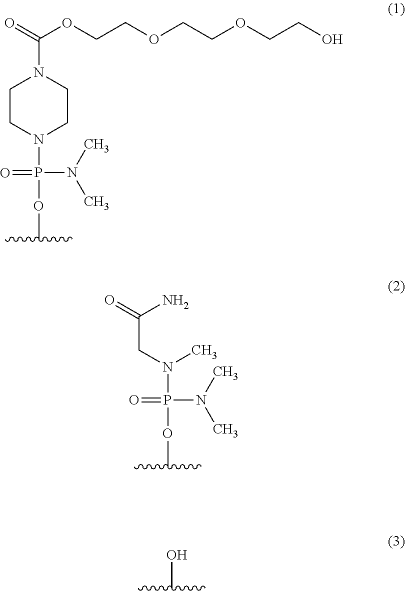

As mentioned above, a functional peptide (e.g., CPP (cell penetrating peptide)) may be bonded to the antisense oligomer without or through a linker. When the functional peptide is bonded to the antisense oligomer through a linker, an additional amino acid may be attached to the functional peptide. In a preferred embodiment, the functional peptide may be bonded to a phosphorodiamidate morpholino oligomer (“PMO”) at 5′-terminal end or 3′-terminal end with bonding to 3′-terminal end being preferred. Also, in a preferred embodiment, the C-terminus of the functional peptide may be bonded to the PMO.

Pharmaceutically Acceptable Salts and Hydrates

Examples of a pharmaceutically acceptable salt of the compound (e.g. antisense oligomer) of the present invention include alkali metal salts (e.g., sodium salt, potassium salt, lithium salt); alkaline earth metal salts (e.g., calcium salt, magnesium salt); metal salts (e.g., aluminum salt, iron salt, zinc salt, copper salt, nickel salt, cobalt salt); ammonium salt; organic amine salts (e.g., t-octylamine salt, dibenzylamine salt, morpholine salt, glucosamine salt, phenylglycine alkyl ester salt, ethylenediamine salt, N-methylglucamine salt, guanidine salt, diethylamine salt, triethylamine salt, dicyclohexylamine salt, N,N′-dibenzylethylenediamine salt, chloroprocaine salt, procaine salt, diethanolamine salt, N-benzyl-phenethylamine salt, piperazine salt, tetramethylammonium salt, tris(hydroxymethyl)aminomethane salt); halogenated hydroacid salts (e.g., hydrofluoride salt, hydrochloride salt, hydrobromide salt, hydroiodide salt); inorganic acid salts (i.e., nitrate salt, perchlorate salt, sulfate salt, phosphate salt); lower alkanesulfonic acid salts (e.g., methanesulfonate salt, trifluoromethanesulfonate salt, ethanesulfonate salt); arylsulfonic acid salts (e.g., benzenesulfonate salt, p-toluenesulfonate salt); organic acid salts (e.g., acetate salt, malate salt, fumarate salt, succinate salt, citrate salt, tartrate salt, oxalate salt, maleate salt); amino acid salts (e.g., glycine salt, lysine salt, arginine salt, ornithine salt, glutamate salt, aspartate salt), etc. These salts may be prepared in any known manner.

A hydrate of the compound (e.g. antisense oligomer) of the present invention may be prepared in any known manner.

### Particular Embodiments

The antisense oligomer of the present invention includes, for example, an oligonucleotide, morpholino oligomer or PNA having a length of the exemplary length range of the antisense oligomer of the present invention or the exemplary length of the antisense oligomer of the present invention. In a particular embodiment the oligonucleotide is an antisense oligomer wherein at least one sugar moiety and/or at least one phosphate bond moiety in the oligonucleotide is modified.

In a particular embodiment, the antisense oligonucleotide of the present invention includes at least one modified sugar moiety that is a ribose in which the —OH group at the 2′-position is substituted with any group selected from the group consisting of OR, R, R′OR, SH, SR, NH₂, NHR, NR₂, N₃, CN, F, Cl, Br and I (wherein R represents alkyl or aryl, and R′ represents alkylene).

In a particular embodiment, the oligonucleotide includes at least one modified phosphate bond moiety selected from the group consisting of a phosphorothioate bond, a phosphorodithioate bond, an alkylphosphonate bond, a phosphoroamidate bond and a boranophosphate bond.

In a particular embodiment, the oligonucleotide is an antisense oligomer that comprises at least one morpholino ring. In a particular embodiment, the antisense is a morpholino oligomer or phosphorodiamidate morpholino oligomer.

In a further embodiment, the antisense oligomer has any one of the groups represented by chemical formulae (1) to (3) shown below at its 5′-terminal end.

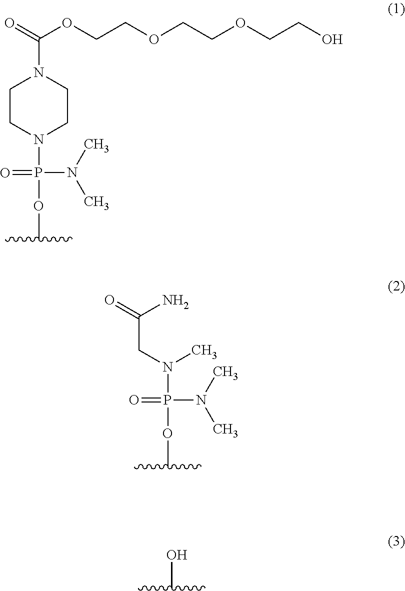

All antisense oligomers tested in the example section may employ any chemical modification as described above.

As mentioned above, a functional peptide (e.g., CPP (cell penetrating peptide)) may be bonded to the antisense oligomer without or through a linker. When the functional peptide is bonded to the antisense oligomer through a linker, an additional amino acid may be attached to the functional peptide. In a preferred embodiment, the functional peptide may be bonded to a phosphorodiamidate morpholino oligomer (“PMO”) at 5′-terminal end or 3′-terminal end.

Pharmaceutical Compositions

According to the second aspect of the invention there is provided a pharmaceutical composition comprising the compound of the present invention (e.g. the antisense oligomer of the present invention) or a pharmaceutically acceptable salt or hydrate thereof as an active ingredient (hereinafter referred to as “the pharmaceutical composition of the present invention”).

In particular embodiments, the pharmaceutical composition comprises any of the aforementioned oligomers or oligonucleotides, or pharmaceutical salts or hydrates thereof, and at least one pharmaceutically acceptable additive and/or carrier.

Suitable formulations for use in the present invention are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

The carrier may serve to promote the delivery of the oligomer to muscle tissue. Such a carrier is not limited in any way as long as it is pharmaceutically acceptable. Suitable examples include cationic carriers (e.g., cationic liposomes, cationic polymers) or viral envelope-based carriers. Examples of cationic liposomes include liposomes formed from 2-O-(2-diethylaminoethyl)carbamoyl-1,3-O-dioleoyl glycerol and a phospholipid as essential constituent members (hereinafter referred to as “liposome A”), Oligofectamine® (Invitrogen), Lipofectin® (Invitrogen), Lipofectamine® (Invitrogen), Lipofectamine 2000® (Invitrogen), DMRIE-C® (Invitrogen), GeneSilencer® (Gene Therapy Systems), TransMessenger® (QIAGEN), TransIT TKO® (Mirus) and Nucleofector II (Lonza).

Among them, preferred is liposome A. Examples of cationic polymers include JetSI® (Qbiogene) and Jet-PEI® (polyethyleneimine, Qbiogene). Examples of viral envelope-based carriers include GenomeOne® (HVJ-E liposomes, Ishihara Sangyo Kaisha, Ltd., Japan). Alternatively, it is also possible to use the pharmaceutical device shown in Japanese Patent No. 2924179 or the cationic carriers shown in JP WO2006/129594 and JP WO2008/096690.

For more details, reference may be made to U.S. Pat. Nos. 4,235,871 and 4,737,323, WO96/14057, “New RRC, Liposomes: A practical approach, IRL Press, Oxford (1990) pages 33-104,” etc.

The pharmaceutical composition of the present invention may optionally comprise a pharmaceutically acceptable additive, in addition to the antisense oligomer of the present invention or a pharmaceutically acceptable salt or hydrate thereof and/or a carrier as described above. Examples of such an additive include an emulsifier aid (e.g., a fatty acid containing 6 to 22 carbon atoms or a pharmaceutically acceptable salt thereof, albumin, dextran), a stabilizing agent (e.g., cholesterol, phosphatidic acid), an isotonizing agent (e.g., sodium chloride, glucose, maltose, lactose, sucrose, trehalose), and a pH adjuster (e.g., hydrochloric acid, sulfuric acid, phosphoric acid, acetic acid, sodium hydroxide, potassium hydroxide, triethanolamine). These additives may be used either alone or in combination. The content of the additive(s) in the pharmaceutical composition of the present invention is reasonably 90% by weight or less, such as 60% by weight or less, and suitably 50% by weight or less.

The pharmaceutical composition of the present invention may be prepared by adding the compound (e.g. antisense oligomer) of the present invention or a pharmaceutically acceptable salt or hydrate thereof to a dispersion of a carrier, followed by adequate stirring. The additive(s) may be added at any appropriate stage, either before or after adding the compound of the present invention or a pharmaceutically acceptable salt or hydrate thereof. Any aqueous solvent may be used for adding the compound of the present invention or a pharmaceutically acceptable salt or hydrate thereof as long as it is pharmaceutically acceptable, and examples include injectable water, injectable distilled water, electrolytic solutions (e.g., physiological saline), and sugar solutions (e.g., glucose solution, maltose solution). Moreover, in this case, conditions including pH and temperature may be selected as appropriate by those skilled in the art.

The pharmaceutical composition of the present invention may be formulated into a solution or a lyophilized formulation thereof. Such a lyophilized formulation may be prepared in a standard manner by freeze-drying the pharmaceutical composition of the present invention in a solution form. For example, the pharmaceutical composition of the present invention in a solution form may be sterilized as appropriate and then dispensed in given amounts into vial bottles, followed by preliminary freezing under conditions of about −40° C. to −20° C. for about 2 hours, primary drying at about 0° C. to 10° C. under reduced pressure and then secondary drying at about 15° C. to 25° C. under reduced pressure. Moreover, in most cases, the vials may be purged with a nitrogen gas and then capped, thereby giving a lyophilized formulation of the pharmaceutical composition of the present invention.

Such a lyophilized formulation of the pharmaceutical composition of the present invention may generally be used after being reconstituted by addition of any appropriate solution (i.e., a reconstituting solution). Examples of such a reconstituting solution include injectable water, physiological saline, and other commonly used infusion solutions. The volume of such a reconstituting solution will vary, e.g., depending on the intended use and is not limited in any way, but it is reasonably 0.5- to 2-fold greater than the solution volume before freeze-drying, or 500 mL or less.

The compound (e.g. antisense oligomer) of the present invention or a pharmaceutically acceptable salt or hydrate thereof contained in the pharmaceutical composition of the present invention may be in the form of a hydrate thereof. Such a hydrate may be prepared in any known manner.

Compositions and methods for the formulation of pharmaceutical compositions are dependent upon a number of criteria, including, but not limited to, route of administration, extent of disease, or dose to be administered.

These compositions may be sterilized by conventional sterilization techniques or may be sterile filtered. The resulting aqueous solutions may be packaged for use as is, or lyophilized, the lyophilized preparation being combined with a sterile aqueous carrier prior to administration. The pH of the preparations typically will be between 3 and 11, suitably between 5 and 9 or between 6 and 8, and most suitably between 7 and 8, such as 7 to 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

The pharmaceutical composition of the present invention may be administered in any pharmaceutically acceptable mode, which may be selected as appropriate for the intended therapeutic method. However, in terms of easy delivery to muscle tissue, preferred are intravenous administration, intraarterial administration, intramuscular administration, subcutaneous administration, oral administration, interstitial administration, percutaneous administration and so on. Moreover, the composition of the present invention may be in any dosage form, and examples include various types of injections, oral formulations, drops, inhalants, ointments, lotions, etc.

The concentration of the compound (e.g. antisense oligomer) of the present invention or a pharmaceutically acceptable salt or hydrate thereof contained in the pharmaceutical composition of the present invention will vary, e.g., depending on the type of carrier, but it is reasonably in the range of 0.1 nM to 100 μM, and suitably in the range of 100 nM to 10 μM. Likewise, the weight ratio of the carrier to the antisense oligomer of the present invention or a pharmaceutically acceptable salt or hydrate thereof contained in the pharmaceutical composition of the present invention (i.e., the carrier/antisense oligomer or pharmaceutically acceptable salt or hydrate thereof ratio) will vary, e.g., depending on the properties of the oligomer and the type of the carrier, but it is reasonably in the range of 0.1 to 100, and suitably in the range of 0.1 to 10.

The compound of the invention, a pharmaceutically acceptable salt or hydrate thereof can be included in a unit formulation such as in a pharmaceutically acceptable carrier or diluent in an amount sufficient to deliver to a subject a therapeutically acceptable amount without causing serious side effects in the subject.

The dose for administration of the pharmaceutical composition of the present invention is desirably adjusted in consideration of the type of the antisense oligomer of the present invention or a pharmaceutically acceptable salt or hydrate thereof contained therein, the intended dosage form, the condition of a subject such as age and body weight, the route of administration, and the nature and severity of a disease. If the subject is a human subject, the daily dose for adults is generally in the range of 0.1 mg to 10 g/kg of bodyweight, such as in the range of 1 mg to 1 g/kg of bodyweight, 20 mg to 120 mg/kg of bodyweight, 30 mg to 100 mg/kg of bodyweight, or 40 mg to 80 mg/kg of bodyweight, calculated as the amount of the antisense oligomer of the present invention or a pharmaceutically acceptable salt or hydrate thereof. This numerical range may vary depending on the type of disease to be targeted, the mode of administration, and/or the type of target molecule. Thus, a dose lower than this range may be sufficient in some cases, or conversely, a dose higher than this range should be required in some cases. Moreover, the pharmaceutical composition of the present invention may be administered once to several times a day or at intervals of one to several days.

In another embodiment, the pharmaceutical composition of the present invention may be a pharmaceutical composition comprising a vector capable of expressing the compound of the present invention (e.g. the antisense oligonucleotide of the present invention) and a carrier as described above. Such an expression vector may be capable of expressing a plurality of antisense oligonucleotides according to the present invention. Such a pharmaceutical composition may optionally comprise a pharmaceutically acceptable additive, as described above. The concentration of the expression vector contained in this pharmaceutical composition will vary, e.g., depending on the type of carrier, but it is reasonably in the range of 0.1 nM to 100 μM, and suitably in the range of 100 nM to 10 μM. The weight ratio of the carrier to the expression vector contained in this pharmaceutical composition (i.e., the carrier/expression vector ratio) will vary, e.g., depending on the properties of the expression vector and the type of the carrier, but it is reasonably in the range of 0.1 to 100, and suitably in the range of 0.1 to 10. Moreover, the content of the carrier contained in this pharmaceutical composition is the same as described above, and procedures for preparation are also the same as described above.

Therapeutic Uses

The compound of the invention, or a pharmaceutically acceptable salt or hydrate thereof or the pharmaceutical composition of the invention comprising these (hereinafter referred to as “Therapeutic agent of the present invention”) may be used in therapy, such as in the treatment of a human.

The Therapeutic agent of the present invention may be provided for use in the prevention or treatment of a metabolic disorder (e.g., obesity, metabolic syndrome, diabetes), an amyotrophic disease, or a muscle wasting disease or a sarcopenic disease. Examples of an amyotrophic disease or a muscle wasting disease include myogenic amyotrophy (e.g., muscular dystrophy (e.g., Duchenne muscular dystrophy, Fukuyama muscular dystrophy, myotonic dystrophy), congenital myopathy, inclusion body myositis), neurogenic amyotrophy (e.g., amyotrophic lateral sclerosis, spinal muscular atrophy, spinal and bulbar muscular atrophy), disuse amyotrophy (e.g., apoplexy-induced disuse syndrome), muscle wasting diseases (e.g., cancer cachexia, sepsis-related amyotrophy) and various types of sarcopenic diseases including age-related skeletal muscle loss (age-related sarcopenia), with muscular dystrophy being particularly suitable.

In some embodiments of the present invention, there is provided a method for prevention or treatment of an amyotrophic disease, a muscle wasting disease or a sarcopenic disease, which comprises administering to a subject in need of prevention or treatment of an amyotrophic disease or a muscle wasting disease with a therapeutically effective amount of the Therapeutic agent of the present invention. In a particular embodiment, the compound (e.g. antisense oligomer) of the present invention or a pharmaceutically acceptable salt or hydrate thereof may be administered to the subject in the form of the pharmaceutical composition of the present invention.

In the context of the present invention, the term “subject” is intended to mean a human subject or a non-human warm-blooded animal, as exemplified by birds and non-human mammals (e.g., cow, monkey, cat, mouse, rat, guinea pig, hamster, pig, dog, rabbit, sheep, horse). The “subject” is preferably a human subject.

In particular embodiments of the present invention, there is provided use of the Therapeutic agent of the present invention in the manufacture of a pharmaceutical composition for the prevention or treatment of an amyotrophic disease or a muscle wasting disease.

In particular embodiments of the present invention, there is provided use of the compound (e.g. antisense oligomer) of the present invention or a pharmaceutically acceptable salt or hydrate thereof in the manufacture of a pharmaceutical composition for the prevention or treatment of an amyotrophic disease or a muscle wasting disease.

In other embodiments of the present invention, there is provided the Therapeutic agent of the present invention for use in the treatment of an amyotrophic disease or a muscle wasting disease.

As described above, the compound of the present invention allows inhibition of myostatin signal transduction at the mRNA level through, for example, induction of exon skipping or mRNA degradation. In one embodiment, the compound of the present invention may be the antisense oligomer of the present invention.

The inhibition of myostatin signal in muscle tissues may be applied to the prevention or treatment of an amyotrophic disease, a muscle wasting disease or a sarcopenic disease. The atrophy of skeletal muscle not only lowers the quality of life of subjects but also accompanies serious systemic complication including malnutrition and respiratory failure and so agents and treatments capable of addressing this medical need are required.

Thus, an amyotrophic disease, a muscle wasting disease or a sarcopenic disease can be prevented or treated when the Therapeutic agent of the present invention is administered to a subject in need of prevention or treatment of an amyotrophic disease, a muscle wasting disease or a sarcopenic disease.

The present invention also provides the Therapeutic agent of the present invention for use in therapy in a subject.

Said therapy may be the prevention or treatment of the disease listed above.

In one embodiment, the therapy may be the prevention or treatment of an amyotrophic disease, a muscle wasting disease or a sarcopenic disease in a subject. In one embodiment, the amyotrophic disease may be Duchenne muscular dystrophy. The subject may be a human subject.

The present invention also provides use of the compound of the present invention or pharmaceutically acceptable salt or hydrate thereof in the manufacture of a medicament (such as a pharmaceutical composition) for preventing or treating an amyotrophic disease, a muscle wasting disease or a sarcopenic disease in a subject. In one embodiment, the amyotrophic disease is Duchenne muscular dystrophy. In one embodiment, the subject is a human.

The dose and administration route may be the same as those listed for the pharmaceutical composition of the present invention.

The present invention also provides a method for treating a disease in a subject, which comprises administering to said subject a therapeutically effective amount of the Therapeutic agent of the present invention. The disease may be a metabolic disorder (e.g., obesity, metabolic syndrome, diabetes), an amyotrophic disease, a muscle wasting disease or a sarcopenic disease. Examples of an amyotrophic disease or a muscle wasting disease include myogenic amyotrophy (e.g., muscular dystrophy (e.g., Duchenne muscular dystrophy, Fukuyama muscular dystrophy, myotonic dystrophy), congenital myopathy, inclusion body myositis), neurogenic amyotrophy (e.g., amyotrophic lateral sclerosis, spinal muscular atrophy, spinal and bulbar muscular atrophy), disuse amyotrophy (e.g., apoplexy-induced disuse syndrome), muscle wasting diseases (e.g., cancer cachexia, sepsis-related amyotrophy) and various types of sarcopenic diseases including age-related skeletal muscle loss (age-related sarcopenia), with muscular dystrophy being particularly suitable. In one embodiment, the therapy may be the prevention or treatment of an amyotrophic disease, a muscle wasting disease or a sarcopenic disease in a subject. In one embodiment, the amyotrophic disease may be Duchenne muscular dystrophy. The subject may be a human subject. The dose and administration route may be the same as those listed for the pharmaceutical composition of the present invention.

Animals

The present invention also provides a genetically manipulated animal that produces a truncated version of ACVR2B protein which lacks part of the intracellular region of ACVR2B (hereinafter referred to as “the GM animal of the present invention”). As used herein, animals may be of any species except for human. Particularly suitable animals are domestic animals such as fish (e.g. tuna), cattle, sheep, goats or pigs.

The person of skill in the art is able to generate such a genetically manipulated animal using standard techniques. For example, the GM animal of the present invention can be produced by administering the compound of the present invention or a pharmaceutical composition of the present invention. For example, the GM animal of the present invention can be produced by CRISPR-CAS9, siRNA, loxP knockout system, TALENs, Zinc fingers (ZFN) or antisense oligomer.

When CRISPR-CAS9 is used, a guide RNA having a sequence complementary to a target sequence of genomic DNA which encode ACVR2B or a part of ACVR2B (e.g. the intracellular region of wild-type ACVR2B) is introduced to a target cell or a host animal, whereby identifying a target sequence to be cleaved. Cas9 (or Cas9-like) protein introduced to the target cell cleaves the double stranded part composed of the genomic DNA and guide RNA. Through a process of repairing the cleavage site, a mutation(s) is caused by deficiency and/or insertion of nucleotides, thereby causing the knock-out of ACVR2B. Examples of the target sequence of genomic DNA includes any sequence of exons, e.g. exons 1 to 11 of ACVR2B. Suitably, the target sequence of genomic DNA includes any sequence of at least one exon selected from the group consisting of exons 5, 6, 7, 8, 9, 10 and 11 or group consisting of exons 5, 6, 7, 8, 9 and 10 of ACVR2B. In one embodiment, the target sequence of genomic DNA includes any sequence of at least one exon selected from the group consisting of exons 5, 6, 7, 9 and 10 of ACVR2B. In another embodiment, the target sequence of genomic DNA includes any sequence of at least one exon selected from the group consisting of exons 5, 6, 9 and 10 of ACVR2B. In another embodiment, the target sequence of genomic DNA includes any sequence of at least one exon selected from the group consisting of exons 5, 6 and 10 of ACVR2B. In another embodiment, the target sequence of genomic DNA includes any sequence of at least one exon selected from the group consisting of exons 5 and 6 of ACVR2B. In another embodiment, the target sequence of genomic DNA includes any sequence of at least one exon selected from the group consisting of exons 7, 8 and 9 of ACVR2B. In another embodiment, the target sequence of genomic DNA includes any sequence of at least one exon selected from the group consisting of exons 7 and 8 of ACVR2B. In another embodiment, the target sequence of genomic DNA is exon 5 of ACVR2B. Yet in another embodiment, the target sequence of genomic DNA is exon 6 of ACVR2B. Yet in another embodiment, the target sequence of genomic DNA is exon 7 of ACVR2B. Yet in another embodiment, the target sequence of genomic DNA is exon 8 of ACVR2B. Yet in another embodiment, the target sequence of genomic DNA is exon 9 of ACVR2B. Yet in another embodiment, the target sequence of genomic DNA is exon 10 of ACVR2B. Yet in another embodiment, the target sequence of genomic DNA is exon 11 of ACVR2B. Yet in another embodiment, the target sequence of genomic DNA includes any sequence of at least one exon selected from the group consisting of exons 7, 8, 9 and 10, or group consisting of exons 5, 6, 7, 8, 9 and 10 of ACVR2B.

Alternatively, introns may be targeted by CRISPR-CAS9. For example, introns 7 and 8 which sandwich exon 8 may be cleaved. When the cleaved sites are being repaired, exon 8 may become absent to generate exon 8-deficient mutant mRNA. Similarly, introns 4 and 5, or introns 5 and 6, or introns 6 and 7, or introns 8 and 9, or introns 9 and 10, or introns 10 and 11 may be targeted to cleave.

When siRNA is used to inhibit the myostatin signal, an siRNA designed to target a sequence of ACVR2B mRNA is introduced to a target cell. When the guide strand of the siRNA thus introduced hybridizes to the targeted sequence, then an endogenous RISC protein in the target cell identifies the double stranded part composed of the guide strand and the targeted mRNA strand and cleaves the targeted sequence of the mRNA. By doing so, the ACVR2B protein level in the GM animal of the present invention is reduced.

It should be noted that all publications cited herein, including prior art documents, patent gazettes and other patent documents, are incorporated herein by reference. The present invention will be further described in more detail below by way of the following illustrative examples, although the present invention is not limited thereto.

### EXAMPLE

### Reference Example 1

### 4-{[(2S,6R)-6-(5-Methyl-2,4-dioxopyrimidin-1-yl)-4-tritylmorpholin-2-yl]methoxy}-4-Oxobutanoic Acid Loaded on Aminoplystyrene Resin

### Step 1: Preparation of 4-{[(2S,6R)-6-(5-methyl-2,4-dioxopyrimidin-1-yl)-4-tritylmorpholin-2-yl]methoxy}-4-oxobutanoic Acid

Under an argon atmosphere, 1-[(2R,6S)-6-(hydroxymethyl)-4-tritylmorpholin-2-yl]-5-methylpyrimidine-2,4-dione (41.11 g) and 4-dimethylaminopyridine (4-DMAP) (15.58 g) were suspended in dichloromethane (850 mL), and succinic anhydride (12.76 g) was then added thereto, followed by stirring at room temperature for 3.5 hours. The reaction solution was extracted with dichloromethane and 1 M aqueous sodium dihydrogen phosphate. The resulting organic layer was washed sequentially with 1 M aqueous sodium dihydrogen phosphate and saturated aqueous sodium chloride. The resulting organic layer was dried over sodium sulfate and concentrated under reduced pressure. To the resulting solid, dichloromethane (600 mL) was added to effect crystallization, followed by filtration. After additional dichloromethane (300 mL) was added, the crystals were stirred for 5 minutes, and then filtered and dried overnight under reduced pressure to obtain the desired product (50.2 g).

### Step 2: Preparation of 4-{[(2S,6R)-6-(5-methyl-2,4-dioxopyrimidin-1-yl)-4-tritylmorpholin-2-yl]methoxy}-4-oxobutanoic Acid Loaded on Aminopolystyrene Resin

4-{[(2S,6R)-6-(5-Methyl-2,4-dioxopyrimidin-1-yl)-4-tritylmorpholin-2-yl]methoxy}-4-oxobutanoic acid (50.2 g) was dissolved in pyridine (dehydrated) (600 mL), followed by addition of 4-DMAP (12.4 g) and 1-(3-dimethylaminopropyl)-3-ethylcarbodiimide hydrochloride (77.6 g). The aminopolystyrene resin Aminomethyl resin (a product of Watanabe Chemical Industries, Ltd., Japan, A00673, 200 to 400 mesh, 1 mmol/g, 1% DVB) (40.5 g) and triethylamine (69.6 mL) were then added to this mixture, followed by shaking at room temperature for 4 days. After the reaction, the resin was collected by filtration. The resulting resin was washed sequentially with pyridine, methanol and dichloromethane, and then dried under reduced pressure. To the resulting resin, tetrahydrofuran (dehydrated) (500 mL), acetic anhydride (104 mL) and 2,6-lutidine (128 mL) were added, followed by shaking at room temperature for 4 hours. The resin was collected by filtration, washed sequentially with pyridine, methanol and dichloromethane, and then dried under reduced pressure to obtain 59.0 g of the desired product.

To determine the loading amount of the desired product, the molar amount of trityl per gram of the resin was measured in a known manner as UV absorbance at 409 nm. The loading amount on the resin was found to be 467.83 μmol/g.
Conditions for UV measurement
Instrument: U-2910 (Hitachi, Ltd., Japan)
Solvent: methanesulfonic acid
Wavelength: 409 nm
ε value: 45000

### Reference Example 2

### 4-{[(2S,6R)-6-(4-Benzamido-2-oxopyrimidin-1-yl)-4-tritylmorpholine-2-yl]methoxy}-4-Oxobutanoic Acid Loaded on Aminopolystyrene Resin

The same procedures as shown in Reference Example 1 were repeated to prepare the titled compound, except that 1-[(2R,6S)-6-(hydroxymethyl)-4-tritylmorpholin-2-yl]-5-methylpyrimidine-2,4-dione used in Step 1 of Reference Example 1 was replaced in this step with N-{1-[(2R,6S)-6-(hydroxymethyl)-4-tritylmorpholin-2-yl]-2-oxo-1,2-dihydropyrimidin-4-yl}benzamide.

To determine the loading amount of the desired product, the molar amount of trityl per gram of the resin was measured in a known manner as UV absorbance at 409 nm. The loading amount on the resin was found to be 460.28 μmol/g.

### Reference Example 3

### 4-{[(2S,6R)-6-(6-Benzamidopurin-9-yl)-4-tritylmorpholine-2-yl]methoxy}-4-oxobutanoic Acid Loaded on Aminopolystyrene Resin

The same procedures as shown in Reference Example 1 were repeated to prepare the titled compound, except that 1-[(2R,6S)-6-(hydroxymethyl)-4-tritylmorpholin-2-yl]-5-methylpyrimidine-2,4-dione used in Step 1 of Reference Example 1 was replaced in this step with N-{9-[(2R,6S)-6-(hydroxymethyl)-4-tritylmorpholin-2-yl]purin-6-yl}benzamide.

To determine the loading amount of the desired product, the molar amount of trityl per gram of the resin was measured in a known manner as UV absorbance at 409 nm. The loading amount on the resin was found to be 425.13 μmol/g.

### Reference Example 4

### 4-{{(2S,6R)-6-{6-(2-Cyanoethoxy)-2-[(2-phenoxyacetyl)amino]purin-9-yl}-4-tritylmorpholin-2-yl}methoxy}-4-oxobutanoic Acid Loaded on Aminopolystyrene Resin

The same procedures as shown in Reference Example 1 were repeated to prepare the titled compound, except that 1-[(2R,6S)-6-(hydroxymethyl)-4-tritylmorpholin-2-yl]-5-methylpyrimidine-2,4-dione used in Step 1 of Reference Example 1 was replaced in this step with N-{6-(2-cyanoethoxy)-9-[(2R,6S)-6-(hydroxymethyl)-4-tritylmorpholin-2-yl]purin-2-yl}-2-phenoxyacetamide.

To determine the loading amount of the desired product, the molar amount of trityl per gram of the resin was measured in a known manner as UV absorbance at 409 nm. The loading amount on the resin was found to be 341.09 μmol/g.

In accordance with the descriptions in Example 1 shown below or in accordance with the procedures described in PCT/JP2015/57180 using a nucleic acid synthesizer (AKTA Oligopilot 10 plus), PMOs were synthesized (the 5′-terminal end is group (3)). Among the synthesized PMOs, those showing skipping activity were listed in Table 1.

TABLE 1

| | | | Molecular | Molecular | |
|---|---|---|---|---|---|
| PMO | | | weight | weight | SEQ ID |
| No. | Sequence name | Nucleotide sequence (5′ to 3′) | (calculated) | (measured) | NO: |
| 1 | ACVR2B_H5_48-71 | CGAGCCTTGATCTCCAGCAGCTGC | 7884.72 | 7884.73 | 12 |
| 2 | ACVR2B_H5_51-74 | CCCCGAGCCTTGATCTCCAGCAGC | 7829.72 | 7829.16 | 13 |
| 3 | ACVR2B_H5_86-109 | TCATGAGCTGGGCCTTCCAGACAC | 7908.75 | 7908.84 | 14 |
| 4 | ACVR2B_H5_119-142 | GGAGTGGGAAGATCTTGACAGCTA | 8076.80 | 8077.00 | 15 |
| 5 | ACVR2B_H5_121-144 | CTGGAGTGGGAAGATCTTGACAGC | 8052.79 | 8052.66 | 16 |
| 6 | ACVR2B_H5_123-146 | ACCTGGAGTGGGAAGATCTTGACA | 8036.79 | 8036.89 | 17 |
| 7 | ACVR2B_H6_126-149 | CTCACCTTGTCATGGAAGGCCGTG | 7939.74 | 7940.09 | 18 |
| 8 | ACVR2B_H7_10-33 | GATGTTCCCCTTGAGGTAATCCGT | 7929.74 | 7929.60 | 19 |
| 9 | ACVR2B_H7_28-51 | CAGTTCGTTCCATGTGATGATGTT | 7959.74 | 7959.31 | 20 |
| 10 | ACVR2B_H7_38-61 | CTACATGACACAGTTCGTTCCATG | 7882.73 | 7882.07 | 21 |
| 11 | ACVR2B_H7_66-89 | TATGAGAGGCCTCGTGACATCGTC | 7963.76 | 7964.32 | 22 |
| 12 | ACVR2B_H7_76-99 | CTCATGCAGGTATGAGAGGCCTCG | 7988.77 | 7988.06 | 23 |
| 13 | ACVR2B_H7_86-109 | AGGGCACATCCTCATGCAGGTATG | 7972.77 | 7972.02 | 24 |
| 14 | ACVR2B_H8_19-42 | GTGAGGTCGCTCTTCAGCAATACA | 7947.75 | 7947.21 | 25 |
| 15 | ACVR2B_H8_21-44 | CTGTGAGGTCGCTCTTCAGCAATA | 7938.74 | 7938.21 | 26 |
| 16 | ACVR2B_H8_26-49 | CACGGCTGTGAGGTCGCTCTTCAG | 7955.74 | 7955.23 | 27 |
| 17 | ACVR2B_H8_45-68 | CCAAGCCAAAGTCAGCCAGCACGG | 7920.77 | 7920.76 | 28 |
| 18 | ACVR2B_H8_73-96 | GGAGGTTTCCCTGGCTCAAATCGA | 7963.76 | 7963.38 | 29 |
| 19 | ACVR2B_H8_78-101 | CCCCTGGAGGTTTCCCTGGCTCAA | 7875.73 | 7875.76 | 30 |
| 20 | ACVR2B_H8_87-110 | CGTGGGTGTCCCCTGGAGGTTTCC | 7962.74 | 7962.81 | 31 |
| 21 | ACVR2B_H8_93-116 | CCTGTCCGTGGGTGTCCCCTGGAG | 7947.73 | 7947.60 | 32 |
| 22 | ACVR2B_H9_56-79 | CAATGCGCAGGAAGGCATCTCTCT | 7932.76 | 7933.08 | 33 |
| 23 | ACVR2B_H9_66-89 | GCATACATGTCAATGCGCAGGAAG | 8005.79 | 8005.48 | 34 |
| 24 | ACVR2B_H10_−1-23 | CAGCATGTACTCATCCACGGGTCC | 7868.74 | 7868.93 | 35 |
| 25 | ACVR2B_H10_49-72 | CCTGCAGCTCCTCCAACGAAGGGT | 7893.75 | 7893.29 | 36 |
| 26 | ACVR2B_H6_16-39 | GCTGAAGATCTCCCGTTCACTCTG | 7874.73 | 7874.32 | 43 |
| 27 | ACVR2B_H6_46-69 | TAGCAGGTTCTCGTGCTTCATGCC | 7905.72 | 7906.34 | 44 |
| 28 | ACVR2B_H7_96-119 | CCACGGCACCAGGGCACATCCTCA | 7847.75 | 7848.04 | 45 |
| 29 | ACVR2B_H8_34-57 | TCAGCCAGCACGGCTGTGAGGTCG | 7989.76 | 7989.50 | 46 |
| 30 | ACVR2B_H8_38-61 | AAAGTCAGCCAGCACGGCTGTGAG | 8006.78 | 8007.05 | 47 |
| 31 | ACVR2B_H8_30-53 | CCAGCACGGCTGTGAGGTCGCTCT | 7940.74 | 7940.81 | 48 |
| 32 | ACVR2B_H8_69-92 | GTTTCCCTGGCTCAAATCGAACAG | 7907.74 | 7907.61 | 49 |
| 33 | ACVR2B_H8_12-35 | CGCTCTTCAGCAATACATTCTTAC | 7817.71 | 7817.90 | 50 |
| 34 | ACVR2B_H8_16-39 | AGGTCGCTCTTCAGCAATACATTC | 7882.73 | 7882.72 | 51 |
| 35 | ACVR2B_H8_42-65 | AGCCAAAGTCAGCCAGCACGGCTG | 7951.77 | 7951.66 | 52 |
| 36 | ACVR2B_H8_24-47 | CGGCTGTGAGGTCGCTCTTCAGCA | 7955.74 | 7955.10 | 53 |
| 37 | ACVR2B_H8_46-69 | GCCAAGCCAAAGTCAGCCAGCACG | 7920.77 | 7920.54 | 54 |
| 38 | ACVR2B_H8_53-76 | TCGAACAGCCAAGCCAAAGTCAGC | 7919.77 | 7919.50 | 55 |
| 39 | ACVR2B_H8_82-105 | GTGTCCCCTGGAGGTTTCCCTGGC | 7922.72 | 7922.98 | 56 |
| 40 | ACVR2B_H8_45-67 | CAAGCCAAAGTCAGCCAGCACGG | 7605.66 | 7605.47 | 57 |
| 41 | ACVR2B_H8_97-120 | GTTACCTGTCCGTGGGTGTCCCCT | 7897.71 | 7897.41 | 58 |
| 42 | ACVR2B_H8_61-84 | GGCTCAAATCGAACAGCCAAGCCA | 7919.77 | 7919.58 | 59 |
| 43 | ACVR2B_H8_89-112 | TCCGTGGGTGTCCCCTGGAGGTTT | 7977.73 | 7977.47 | 60 |
| 44 | ACVR2B_H8_15-38 | GGTCGCTCTTCAGCAATACATTCT | 7873.72 | 7873.52 | 61 |
| 45 | ACVR2B_H8_−5-19 | ATTCTTACTTTTAAAGTCCCTGTG | 7878.72 | 7878.48 | 62 |
| 46 | ACVR2B_H8_−2-22 | TACATTCTTACTTTTAAAGTCCCT | 7822.70 | 7822.60 | 63 |
| 47 | ACVR2B_H8_18-41 | TGAGGTCGCTCTTCAGCAATACAT | 7922.74 | 7922.70 | 64 |
| 48 | ACVR2B_H8_14-37 | GTCGCTCTTCAGCAATACATTCTT | 7848.71 | 7848.54 | 65 |
| 49 | ACVR2B_H8_22-45 | GCTGTGAGGTCGCTCTTCAGCAAT | 7954.74 | 7954.19 | 66 |
| 50 | ACVR2B_H8_23-46 | GGCTGTGAGGTCGCTCTTCAGCAA | 7979.75 | 7979.16 | 67 |
| 51 | ACVR2B_H8_13-36 | TCGCTCTTCAGCAATACATTCTTA | 7832.71 | 7832.30 | 68 |
| 52 | ACVR2B_H8_20-43 | TGTGAGGTCGCTCTTCAGCAATAC | 7938.74 | 7938.70 | 69 |
| 53 | ACVR2B_H8_17-40 | GAGGTCGCTCTTCAGCAATACATT | 7922.74 | 7922.78 | 70 |

### Example 1

4-{[(2S,6R)-6-(5-Methyl-2,4-dioxopyrimidin-1-yl)-4-tritylmorpholin-2-yl]methoxy}-4-oxobutanoic acid loaded on aminopolystyrene resin (Reference Example 1) or 4-{[(2S,6R)-6-(4-benzamido-2-oxopyrimidin-1-yl)-4-tritylmorpholin-2-yl]methoxy}-4-oxobutanoic acid loaded on aminopolystyrene resin (Reference Example 2) or 4-{[(2S,6R)-6-(6-benzamidopurin-9-yl)-4-tritylmorpholin-2-yl]methoxy}-4-oxobutanoic acid loaded on aminopolystyrene resin (Reference Example 3) or 4-{{(2S,6R)-6-{6-(2-cyanoethoxy)-2-[(2-phenoxyacetyl)amino]purin-9-yl}-4-tritylmorpholin-2-yl}methoxy}-4-oxobutanoic acid loaded on aminopolystyrene resin (Reference Example 4), each corresponding to the 5′-terminal base, was filled in an amount of 0.1 g into a reaction vessel equipped with a filter to initiate the following synthesis cycles using a peptide synthesizer (FOCUS). To give the nucleotide sequence of each compound indicated in Table 1, a desired morpholino monomer compound was added in each coupling cycle (see Table 2 below).

TABLE 2

| | | Volume | Time | Number |
|---|---|---|---|---|
| Step | Reagent | (mL/run) | (min/run) | of runs |
| 1 | Deblocking solution | 1.8 to 3 | 0.1 to 2 | 3 to 8 |
| 2 | Neutralizing solution | 2 to 10 | 1 | 3 |
| 3 | Dichloromethane | 2 to 10 | — | 5 |
| 4 | Activator solution | 1.8 to 3 | — | 1 |
| 5 | Monomer solution | 1 to 1.5 | — | 1 |
| 6 | Activator solution | 0.9 to 1.4 | — | 1 |
| 7 | Coupling reaction with | | 120 to 180 | |
| | the reagents charged | | | |
| | in Steps 5 and 6 | | | |
| 8 | Dichloromethane | 2 to 10 | — | 5 |
| 9 | Capping solution | 2 to 3 | 2 | 2 |
| 10 | Dichloromethane | 2 to 10 | — | 5 |

It should be noted that the deblocking solution used was prepared by dissolving a mixture of trifluoroacetic acid (2 equivalents) and triethylamine (1 equivalent) at a concentration of 3% (w/v) in a dichloromethane solution containing 1% (v/v) ethanol and 10/a (v/v) 2,2,2-trifluoroethanol. The neutralizing solution used was prepared by dissolving N,N-diisopropylethylamine at a concentration of 5% (v/v) in a dichloromethane solution containing 25% (v/v) 2-propanol. The activator solution used was a 1,3-dimethyl-2-imidazolidinone solution containing 20% (v/v) N,N-diisopropylethylamine. The monomer solution used was prepared by dissolving a morpholino monomer compound at a concentration of 0.20 M in tetrahydrofuran. The capping solution used was prepared by dissolving acetic anhydride at 10% (v/v) and 2,6-lutidine at 15% (v/v) in dichloromethane.

The aminopolystyrene resin loaded with PMO synthesized as above was collected from the reaction vessel and dried at 30° C. for 2 hours or longer under reduced pressure. The dried PMO loaded on the aminopolystyrene resin was charged into a reaction vessel and 5 mL of 28% aqueous ammonia-ethanol (1/3) was added thereto, followed by standing at 55° C. for 16 hours. The aminopolystyrene resin was separated by filtration and washed with 3 mL of water-acetonitrile (1/1). After the resulting filtrate was mixed with ethanol (3 mL) and diethyl ether (35 mL), the mixture was centrifuged and then decanted to remove the supernatant, and the residue was dried under reduced pressure. The resulting residue was dissolved in 10 mL of a mixed solvent containing 20 mM aqueous ammonium acetate and acetonitrile (4/1), and then purified by reversed-phase HPLC. The conditions used are as indicated in Table 3 blow.

TABLE 3

| | |
|---|---|
| Column | XBridge 5 μm C18 (Waters, ϕ19 × 50 mm, 1 CV = |
| | 14 mL) |
| Flow rate | 10 mL/minute |
| Column | room temperature |
| temperature | |
| Solution A | 20 mM aqueous ammonium acetate |
| Solution B | CH₃CN |
| Gradient | (B) conc. 20% → 50%/10 CV |

CV: column volume

The fractions were each analyzed to collect the desired product. The resulting solution was mixed with 0.1 M aqueous hydrochloric acid (4 mL) and allowed to stand for 2 hours. After the reaction, 1 M aqueous sodium hydroxide (0.4 mL) was added to neutralize the mixture, which was then filtered through a membrane filter (0.22 μm).

The resulting aqueous solution containing the desired product was made alkaline with 1 M aqueous sodium hydroxide (0.4 mL) and purified through an anion exchange resin column. The conditions used are as indicated in Table 4 below.

TABLE 4

| | |
|---|---|
| Column | Source 15Q (GE Healthcare, ϕ16 × 97 mm, 1 CV = |
| | 19.5 mL) |
| Flow rate | 10 mL/minute |
| Column | room temperature |
| temperature | |
| Solution A | 10 mM aqueous sodium hydroxide |
| Solution B | 10 mM aqueous sodium hydroxide, |
| | 1M aqueous sodium chloride |
| Gradient | (B) conc. 5% → 50%/20 CV |

The fractions were each analyzed (by HPLC) to obtain the desired product as an aqueous solution. The resulting aqueous solution was neutralized with 0.1 M phosphate buffer (pH 6.0) and then desalted by reversed-phase HPLC under the conditions shown in Table 5 below.

TABLE 5

| | |
|---|---|
| Column | YMC GEL C4 HG 10 μm (YMC, ϕ10 × 35 mm, 1 CV = |
| | 2.7 mL) |
| Flow rate | 10 mL/minute |
| Column | room temperature |
| temperature | |
| Solution A | water |
| Solution B | CH₃CN |
| Gradient | (B) conc. 0% → 50%/10 CV |

The desired product was collected and concentrated under reduced pressure. The resulting residue was dissolved in water and freeze-dried to obtain the desired compound as a white flocculent solid. The calculated and measured values of ESI-TOF-MS are shown in Tables 1.

### Example 2

### Synthesis of Antisense Oligomer-Peptide Conjugate (PMO-Peptide Conjugate)

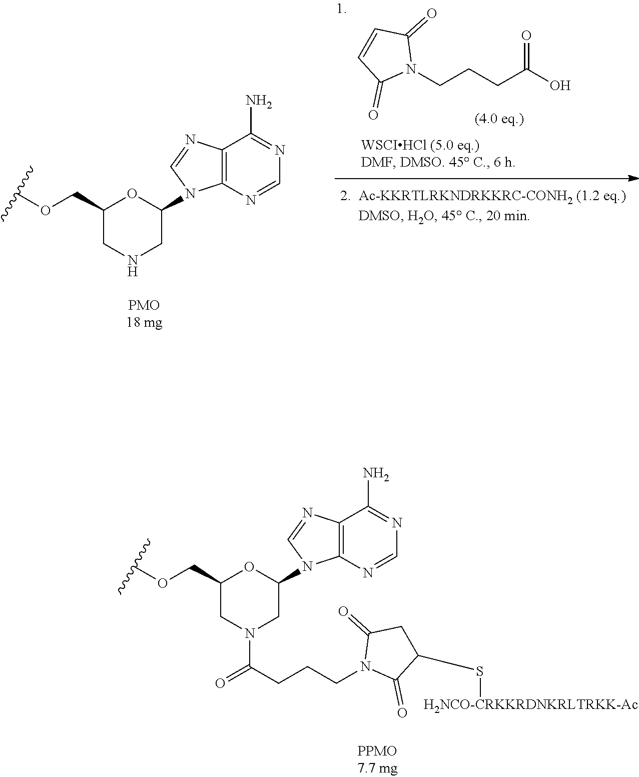

The PMO (PMO No. 15, 18.1 mg, 1.0 eq.) was dissolved in DMSO (284.0 μL) and DMF (17.5 μL). To the solution was added a mixture of 4-maleimidobutyric acid (1.7 mg, 4.0 eq.) and WSCI·HCl (2.2 mg, 5.0 eq.) in DMSO (23.2 μL) and DMF (22.2 μL). The reaction mixture was stirred at 45° C. for 6 h. After the reaction had almost reached completion, CH₂Cl₂ (13.2 mL) was added to the mixture to give a precipitate. The precipitate was collected by centrifugation, followed by drying in a vacuum. The dried precipitate was re-dissolved in DMSO (175.0 μL) and H₂O (52.7 μL). To the resulting solution was added hLIMK (Ac-KKRTLRKNDRKKRC—CONH₂, 5.1 mg, 1.2 eq.) (SEQ ID NO: 112) and the mixture was stirred at 45° C. for 30 min. After confirmation of the completion of the reaction by HPLC analysis, the reaction was terminated by the addition of acetonitrile solution (10% in H₂O; 400 μL). The solution was diluted with water (ca. 40 mL) and the desired product was purified by cation exchange chromatography. The conditions used are shown in Table 6 below.

TABLE 6

| | |
|---|---|
| Column | Source 15S (GE Healthcare, ϕ16 × 50 mm, 1 CV = |
| | 10 mL) |
| Flow rate | 10 mL/min |
| Column | room temperature |
| temperature | |
| Solution A | 25 mM KH₂PO₄ with 25% MeCN (pH 3.5) |
| Solution B | 25 mM KH₂PO₄ with 25% MeCN (pH 3.5) and |
| | 1.5M KCl |
| Gradient | (B) conc. 0% → 90%/30 CV |

CV: column volume

The fractions were analyzed by HPLC and the appropriate fractions were collected. The aqueous solution was diluted seven times with water and then desalted by reversed-phase HPLC under the conditions shown in Table 7 below.

TABLE 7

| | |
|---|---|
| Column | AMBERCHROM_CG300m_10 mm × 30 mm (1 CV = |
| | 2.4 mL) |
| Flow rate | 5 mL/min |
| Column | room temperature |
| temperature | |
| Solution A | water |
| Solution B | MeOH |
| Gradient | (B) conc. 0% → 90%/30 CV |

The desired product was collected and concentrated under reduced pressure. The resulting residue was dissolved in water and freeze-dried to give the desired compound (designated as PPMO No. 1) as a white flocculent solid (7.7 mg, 34.0% yields).

The molecular weight of the obtained compound (PPMO No. 1) was determined by using ESI-TOF-MS (calculated: 9973.92, observed: 9973.54).

### Example 3

Evaluation of the Skipping Activity of Antisense Oligomers

Transfection of PMO into the Cells

Into 3×10⁵ RD cells (human rhabdomyosarcoma cell line), the antisense oligomers shown in Table 1 were each transfected at 10 or 30 μM using Nucleofector II (Lonza) and an Amaxa Cell Line Nucleofector Kit. The pulse program used was T-030.

After transfection, the cells were cultured in the wells of 6-well plates under 5% CO₂ in Dulbecco's Modified Eagle's Medium (DMEM) (Sigma-Aldrich) containing 10% fetal bovine serum (FBS) (Sigma-Aldrich). Three days after transfection, the culture medium was replaced with serum-free DMEM containing 0.4 ng/mL recombinant myostatin (R&D Systems) and the cells were cultured for another 2 hours at 37° C. under 5% CO₂.

Transfection of Phosphorothioate (PS) Oligonucleotides into the Cells

The day before transfection, RD cells were seeded in 24-well plates at a density of 3×10⁴ cells/well and cultured under 5% CO₂ in DMEM (Sigma-Aldrich) containing 10% FBS (Sigma-Aldrich). The next day, the antisense oligomers of 2′-O-methyl phosphorothioate (PS) oligonucleotides (JbioS) shown in Table 8 were each transfected at 10 or 30 nM using Lipofectamine 3000 Transfection Reagent (Thermo Scientific). Cells were cultured for three days after transfection.

TABLE 8

| | | | Molecular | Molecular | |
|---|---|---|---|---|---|
| PS | | | weight | weight | SEQ ID |
| No. | Sequence name | Nucleotide sequence (5′ to 3′) | (calculated) | (measured) | NO: |
| 1 | ACVR2B_054 | CAGCUGCAGUGGCUUCAGGCCCAC | 8347 | 8352.5791 | 71 |
| 2 | ACVR2B_055 | UGAUCUCCAGCAGCUGCAGUGGCU | 8349 | 8355.2591 | 72 |
| 3 | ACVR2B_056 | CCCCGAGCCUUGAUCUCCAGCAGC | 8267 | 8270.6786 | 73 |
| 4 | ACVR2B_060 | GUCAUUCAUGAGCUGGGCCUUCCA | 8310 | 8314.0250 | 74 |
| 5 | ACVR2B_061 | CAGCUACAAAGUCAUUCAUGAGCU | 8325 | 8327.7387 | 75 |
| 6 | ACVR2B_062 | AAGAUCUUGACAGCUACAAAGUCA | 8372 | 8376.2644 | 76 |
| 7 | ACVR2B_063 | CUGGAGUGGGAAGAUCUUGACAGC | 8476 | 8480.5301 | 77 |
| 8 | ACVR2B_006 | AACUGUAGCAGGUUCUCGUGCUUC | 8311 | 8313.9651 | 78 |
| 9 | ACVR2B_014 | CUUGAGGUAAUCCGUGAGGGAGCC | 8452 | 8455.6050 | 79 |
| 10 | ACVR2B_015 | GAUGUUCCCCUUGAGGUAAUCCGU | 8311 | 8317.1643 | 80 |
| 11 | ACVR2B_016 | CCAUGUGAUGAUGUUCCCCUUGAG | 8311 | 8313.8275 | 81 |
| 12 | ACVR2B_017 | CAGUUCGUUCCAUGUGAUGAUGUU | 8313 | 8315.9555 | 82 |
| 13 | ACVR2B_018 | UACAUGACACAGUUCGUUCCAUGU | 8279 | 8281.5703 | 83 |
| 14 | ACVR2B_019 | CGUCUCUGCUACAUGACACAGUUC | 8254 | 8258.0108 | 84 |
| 15 | ACVR2B_020 | CUCGUGACAUCGUCUCUGCUACAU | 8231 | 8235.1182 | 85 |
| 16 | ACVR2B_021 | UAUGAGAGGCCUCGUGACAUCGUC | 8373 | 8375.2037 | 86 |
| 17 | ACVR2B_022 | CUCAUGCAGGUAUGAGAGGCCUCG | 8412 | 8416.8200 | 87 |
| 18 | ACVR2B_023 | AGGGCACAUCCUCAUGCAGGUAUG | 8396 | 8399.2399 | 88 |
| 19 | ACVR2B_024 | CCACGGCACCAGGGCACAUCCUCA | 8313 | 8318.7891 | 89 |
| 20 | ACVR2B_030 | GUGAGGUCGCUCUUCAGCAAUACA | 8357 | 8361.8757 | 90 |
| 21 | ACVR2B_031 | AGCACGGCUGUGAGGUCGCUCUUC | 8365 | 8369.4975 | 91 |
| 22 | ACVR2B_032 | AAGUCAGCCAGCACGGCUGUGAGG | 8474 | 8475.5442 | 92 |
| 23 | ACVR2B_033 | GCCAAGCCAAAGUCAGCCAGCACG | 8400 | 8402.1792 | 93 |
| 24 | ACVR2B_034 | AAUCGAACAGCCAAGCCAAAGUCA | 8393 | 8397.0875 | 94 |
| 25 | ACVR2B_035 | CCUGGCUCAAAUCGAACAGCCAAG | 8362 | 8366.7717 | 95 |
| 26 | ACVR2B_036 | GGAGGUUUCCCUGGCUCAAAUCGA | 8373 | 8375.6335 | 96 |
| 27 | ACVR2B_037 | GUGUCCCCUGGAGGUUUCCCUGGC | 8318 | 8322.6302 | 97 |
| 28 | ACVR2B_038 | CUGUCCGUGGGUGUCCCCUGGAGG | 8397 | 8399.1436 | 98 |
| 29 | ACVR2B_064 | AGCCAUGUACCGUCUCGUGCCUAC | 8269 | 8273.6442 | 99 |
| 30 | ACVR2B_065 | CACCUCAGGAGCCAUGUACCGUCU | 8292 | 8297.9971 | 100 |
| 31 | ACVR2B_068 | UCUCUGGAAGUUGAUGGCUCCCUC | 8287 | 8287.8070 | 101 |
| 32 | ACVR2B_069 | GAAGGCAUCUCUCUGGAAGUUGAU | 8398 | 8400.5961 | 102 |
| 33 | ACVR2B_070 | CAAUGCGCAGGAAGGCAUCUCUCU | 8356 | 8360.2476 | 103 |
| 34 | ACVR2B_071 | GCAUACAUGUCAAUGCGCAGGAAG | 8443 | 8447.4672 | 104 |
| 35 | ACVR2B_073 | CCCACAGCACCAACCCCAUGGCAU | 8257 | 8262.1851 | 105 |
| 36 | ACVR2B_074 | GACACAAGCUCCCACAGCACCAAC | 8304 | 8308.9227 | 106 |
| 37 | ACVR2B_075 | CUUGCAGCGAGACACAAGCUCCCA | 8338 | 8342.7494 | 107 |
| 38 | ACVR2B_076 | CGUCUGCAGCCUUGCAGCGAGACA | 8371 | 8376.4996 | 108 |
| 39 | ACVR2B_039 | GCAGCAUGUACUCAUCCACGGGUC | 8332 | 8336.2184 | 109 |
| 40 | ACVR2B_043 | UCCAACGAAGGGUGCUGGCCAAUC | 8395 | 8401.4766 | 110 |
| 41 | ACVR2B_044 | CUGCAGCUCCUCCAACGAAGGGUG | 8371 | 8377.0700 | 111 |

Gymnotic Delivery of a PMO-Peptide Conjugate into the Cells

The day before transfection, RD cells were seeded in 24-well plates at a density of 4×10⁴ cells/well and cultured under 5% CO₂ in DMEM (Sigma-Aldrich) containing 10% FBS (Sigma-Aldrich). The next day, the antisense oligomer-peptide conjugate (PMO-peptide conjugate) synthesized in Example 2 was added to the medium. Cells were cultured for three days after the addition of the PMO-peptide conjugate.

RNA Extraction

The cells were washed once with PBS (Nissui Pharmaceutical), then 350 μL of Buffer RLT (Qiagen) containing 1% 2-mercaptoethanol (Nacalai Tesque) was added to the cells, and the cells were lysed by being allowed to stand at room temperature for a few minutes. The cell lysate was collected into a QIAshredder homogenizer (Qiagen) and centrifuged at 20,400×g for 2 minutes to prepare a homogenate. The total RNA was extracted in accordance with the manufacture's instruction of an RNeasy Mini Kit (Qiagen). The concentration of the extracted total RNA was measured with a NanoDrop ND-1000 spectrophotometer (Thermo Fisher Scientific).

Measurement of Skipping Efficiency

The extracted total RNA (150 ng) was used as a template to perform one-step RT-PCR with a QIAGEN OneStep RT-PCR Kit (Qiagen). A reaction solution was prepared in accordance with the protocol attached to the kit. The thermal cycler used was TaKaRa PCR Thermal Cycler Dice Touch (Takara Bio). The RT-PCR program used is as shown below.
50° C. for 30 min: reverse transcription reaction
95° C. for 15 min: polymerase activation, reverse transcriptase inactivation
[94° C. for 30 sec; 62° C. for 30 sec; 72° C. for 50 sec]×32 cycles: PCR
72° C. for 7 min: final elongation reaction

The nucleotide sequences of the forward and reverse primers used for RT-PCR are as shown below.

For detection of exon 5 skipping:

Forward primer
(SEQ ID NO: 37)
5′-TGCTACGATAGGCAGGAGTG-3′
Reverse primer
(SEQ ID NO: 38)
5′-AGCAGGTTCTCGTGCTTCAT-3′

For detection of exon 6, 7, or 8 skipping:

Forward primer
(SEQ ID NO: 39)
5′-CATGTGGACATCCATGAGGA-3′
Reverse primer
(SEQ ID NO: 40)
5′-GAGACACAAGCTCCCACAGC-3′

For detection of exon 9 or 10 skipping:

Forward primer
(SEQ ID NO: 41)
5′-TCTATTGCCCACAGGGACTT-3′
Reverse primer
(SEQ ID NO: 42)
5′-GAGCCTCTGCATCATGGTC-3′

The above PCR reaction solution (1 μL) was analyzed using a Bioanalyzer (Agilent). The polynucleotide molarity “A” of the PCR amplicon with exon skipping, i.e. truncated ACVR2B cDNA, and the polynucleotide molarity “B” of the wild-type PCR amplicon, i.e. wild-type ACVR2B cDNA, were measured. Based on these measured values of “A” and “B”, the skipping efficiency was determined according to the following equation.

$$Skipping efficiency ( \% ) = A / ( A + B ) ⨯ 100$$

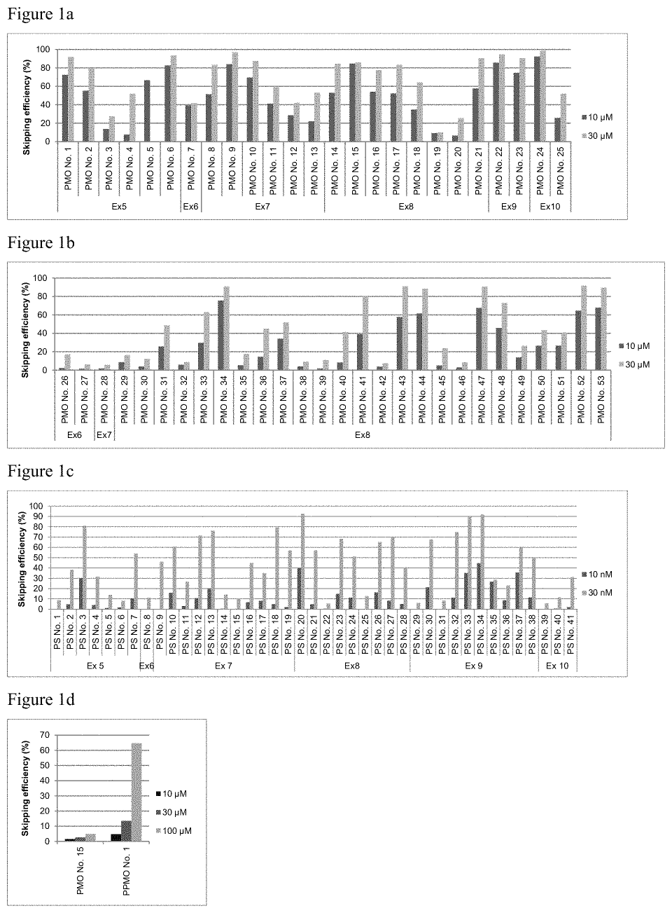

The results obtained are shown in FIGS. 1a-d.

FIGS. 1a-d indicated that the antisense oligomer and the antisense oligomer-peptide conjugate of the present invention caused exon skipping.

### Example 4

Measurement of Dominant-Negative Activity

8×10³ HEK-293 cells (human embryonic kidney cell line) were seeded in 96-well white plates (Thermo Fisher Scientific) and cultured at 37° C. under 5% CO₂ in Minimum Essential Medium Eagle (Sigma-Aldrich) containing 10% FBS. The next day, 10 ng of pBApo-CMV (Takara Bio) harboring DNA encoding each exon (exon 5, 6, 7, 8, 9 or 10) skipping product (Eurofins Genomics) and 14 ng of luciferase reporter DNA (Cignal SMAD Reporter Assay Kit, Qiagen) were co-transfected into the cells using Lipofectamine LTX (Thermo Fisher Scientific). Two days after transfection, recombinant myostatin (R&D Systems) diluted in serum-free Minimum Essential Medium Eagle was added to the medium at a final concentration of 10 ng/mL. After an additional 24 hours of culture, luciferase reporter assay was performed using Dual-Glo Luciferase Assay System (Promega) according to the manufacture's instruction. The luciferase activity was measured using a luminometer, Tecan infinite F200 PRO (Tecan).

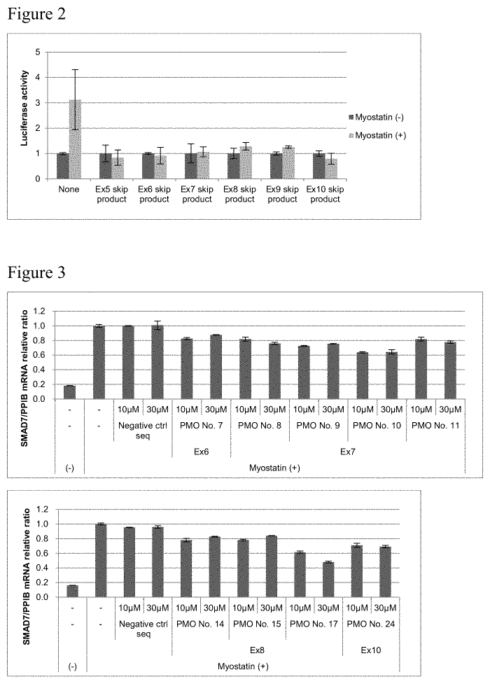

The results obtained are shown in FIG. 2.

As shown in FIG. 2, the luciferase activity induced by addition of myostatin was almost completely suppressed in the cells transfected with DNA encoding each exon skipping product. FIG. 2 indicated that any of the exon skipping (exon 5, 6, 7, 8, 9 and 10) could effectively suppress the myostatin signal.

### Example 5

Measurement of Myostatin Signal

The extracted total RNA (360 ng) was used as a template to perform RT reaction with a High Capacity cDNA Reverse Transcription Kit (Thermo Fisher Scientific). Preparation of the reaction solution and the thermal conditions were followed to the manufacture's instruction of the kit. The thermal cycler used was TaKaRa PCR Thermal Cycler Dice Touch (Takara Bio).

The solution of the RT reaction (0.6 μL) was used as a template to perform qPCR with TaqMan Gene Expression Master Mix (Thermo Fisher Scientific) and TaqMan Gene Expression Assays for SMAD7 and PPIB (Thermo Fisher Scientific). The instrument used for qPCR was QuantStudio 6 Flex Systems (Thermo Fisher Scientific).

The results obtained are shown in FIG. 3.

As shown in FIG. 3, the expression of SMAD7 gene increased 6-fold by stimulation with myostatin. The antisense oligomers of the present invention suppressed the increased expression of SMAD7 gene.

These antisense oligomers have no homology to the SMAD7 gene in the BLAST analysis. Therefore, these SMAD7 gene expression inhibitory activities are specific to myostatin signal inhibitory activity.

SEQUENCE IDENTIFIERS:
SEQ ID NO: 1
GACCCUGGGCCUCCACCACCAUCCCCUCUGGUGGGCCUGAAGCCACUGCA
GCUGCUGGAGAUCAAGGCUCGGGGGCGCUUUGGCUGUGUCUGGAAGGCCC
AGCUCAUGAAUGACUUUGUAGCUGUCAAGAUCUUCCCACUCCAG
SEQ ID NO: 2
GACAAGCAGUCGUGGCAGAGUGAACGGGAGAUCUUCAGCACACCUGGCAU
GAAGCACGAGAACCUGCUACAGUUCAUUGCUGCCGAGAAGCGAGGCUCCA
ACCUCGAAGUAGAGCUGUGGCUCAUCACGGCCUUCCAUGACAAG
SEQ ID NO: 3
GGCUCCCUCACGGAUUACCUCAAGGGGAACAUCAUCACAUGGAACGAACU
GUGUCAUGUAGCAGAGACGAUGUCACGAGGCCUCUCAUACCUGCAUGAGG
AUGUGCCCUGGUGCCGUGGCGAGGGCCACAAGCCGUCUAUUGCCCACAG
SEQ ID NO: 4
GGACUUUAAAAGUAAGAAUGUAUUGCUGAAGAGCGACCUCACAGCCGUGC
UGGCUGACUUUGGCUUGGCUGUUCGAUUUGAGCCAGGGAAACCUCCAGGG
GACACCCACGGACAG
SEQ ID NO: 5
GUAGGCACGAGACGGUACAUGGCUCCUGAGGUGCUCGAGGGAGCCAUCAA
CUUCCAGAGAGAUGCCUUCCUGCGCAUUGACAUGUAUGCCAUGGGGUUGG
UGCUGUGGGAGCUUGUGUCUCGCUGCAAGGCUGCAGACG
SEQ ID NO: 6
GACCCGUGGAUGAGUACAUGCUGCCCUUUGAGGAAGAGAUUGGCCAGCAC
CCUUCGUUGGAGGAGCUGCAGGAGGUGGUGGUGCACAAGAAGAUGAGGCC
CACCAUUAAAGAUCACUGGUUGAAACACCCG
SEQ ID NO: 7 (exon 11 including terminal UAA stop
codon)
GGCCUGGCCCAGCUUUGUGUGACCAUCGAGGAGUGCUGGGACCAUGAUGC
AGAGGCUCGCUUGUCCGCGGGCUGUGUGGAGGAGCGGGUGUCCCUGAUUC
GGAGGUCGGUCAACGGCACUACCUCGGACUGUCUCGUUUCCCUGGUGACC
UCUGUCACCAAUGUGGACCUGCCCCCUAAAGAGUCAAGCAUCUAA
SEQ ID NO: 8:
GGCUCGGCUCCGUGGCCGCCGCUCAGGAGCCAUUUUGGACUCGGUUCAGC
UCCCCUCCCCCCCACCCCUCCCCCCGUUCAUGGCCCCUCCGGACUCGGCC
CCUGCGCCCGGGGCCCGGGCCCAGCCCCGCCGCGCUAUGCCUGAGUCGGG
CGCGCCCCGGCCCGUGCCCCGCCGCCGCCCCCCGGCCCCCGCGUCGCCCC
GGAGCCCGGGCCGCAGCCUGCGCCGCCCGCAGCGGCCCUGAGCCCGGCCC
CGCCGACCGGCCCUUGGAGCCCGAACGCUGCUCGGGGACGAAGGCGCAGG
AAGCGCGCAGGGAACGAGACCGAAGGAAGGAGCGGGAAGGAGAGCGCAGC
CGCCGCCUGGCCCUGCGCGCCCCGGGAGCGCCGUGCGGCCCUGCCCGCGG
GCUCCGGGUGUGCGCGGGGCGGCGCCGCGGAACAUGACGGCGCCCUGGGU
GGCCCUCGCCCUCCUCUGGGGAUCGCUGUGCGCCGGCUCUGGGCGUGGGG
AGGCUGAGACACGGGAGUGCAUCUACUACAACGCCAACUGGGAGCUGGAG
CGCACCAACCAGAGCGGCCUGGAGCGCUGCGAAGGCGAGCAGGACAAGCG
GCUGCACUGCUACGCCUCCUGGCGCAACAGCUCUGGCACCAUCGAGCUCG
UGAAGAAGGGCUGCUGGCUAGAUGACUUCAACUGCUACGAUAGGCAGGAG
UGUGUGGCCACUGAGGAGAACCCCCAGGUGUACUUCUGCUGCUGUGAAGG
CAACUUCUGCAACGAACGCUUCACUCAUUUGCCAGAGGCUGGGGGCCCGG
AAGUCACGUACGAGCCACCCCCGACAGCCCCCACCCUGCUCACGGUGCUG
GCCUACUCACUGCUGCCCAUCGGGGGCCUUUCCCUCAUCGUCCUGCUGGC
CUUUUGGAUGUACCGGCAUCGCAAGCCCCCCUACGGUCAUGUGGACAUCC
AUGAGGACCCUGGGCCUCCACCACCAUCCCCUCUGGUGGGCCUGAAGCCA
CUGCAGCUGCUGGAGAUCAAGGCUCGGGGGCGCUUUGGCUGUGUCUGGAA
GGCCCAGCUCAUGAAUGACUUUGUAGCUGUCAAGAUCUUCCCACUCCAGG
ACAAGCAGUCGUGGCAGAGUGAACGGGAGAUCUUCAGCACACCUGGCAUG
AAGCACGAGAACCUGCUACAGUUCAUUGCUGCCGAGAAGCGAGGCUCCAA
CCUCGAAGUAGAGCUGUGGCUCAUCACGGCCUUCCAUGACAAGGGCUCCC
UCACGGAUUACCUCAAGGGGAACAUCAUCACAUGGAACGAACUGUGUCAU
GUAGCAGAGACGAUGUCACGAGGCCUCUCAUACCUGCAUGAGGAUGUGCC
CUGGUGCCGUGGCGAGGGCCACAAGCCGUCUAUUGCCCACAGGGACUUUA
AAAGUAAGAAUGUAUUGCUGAAGAGCGACCUCACAGCCGUGCUGGCUGAC
UUUGGCUUGGCUGUUCGAUUUGAGCCAGGGAAACCUCCAGGGGACACCCA
CGGACAGGUAGGCACGAGACGGUACAUGGCUCCUGAGGUGCUCGAGGGAG
CCAUCAACUUCCAGAGAGAUGCCUUCCUGCGCAUUGACAUGUAUGCCAUG
GGGUUGGUGCUGUGGGAGCUUGUGUCUCGCUGCAAGGCUGCAGACGGACC
CGUGGAUGAGUACAUGCUGCCCUUUGAGGAAGAGAUUGGCCAGCACCCUU
CGUUGGAGGAGCUGCAGGAGGUGGUGGUGCACAAGAAGAUGAGGCCCACC
AUUAAAGAUCACUGGUUGAAACACCCGGGCCUGGCCCAGCUUUGUGUGAC
CAUCGAGGAGUGCUGGGACCAUGAUGCAGAGGCUCGCUUGUCCGCGGGCU
GUGUGGAGGAGCGGGUGUCCCUGAUUCGGAGGUCGGUCAACGGCACUACC
UCGGACUGUCUCGUUUCCCUGGUGACCUCUGUCACCAAUGUGGACCUGCC
CCCUAAAGAGUCAAGCAUCUAAGCCCAGGACAUGAGUGUCUGUCCAGACU
CAGUGGAUCUGAAGAAAAAAGGAAAAAAAGUUGUGUUUUGUUUUGGAAAU
CCCAUAAAACCAACAAACACAUAAAAUGCAGCUGCUAUUUUACCUUGACU
UUUUAUUAUUAUUAUUAUAAUUAUUAUAAUUAUUAUUAUUAAUAUUAUUU
UUUGGAUUGGAUCAGUUUUUACCAGCAUAUUGCUCUACUGUAUCACAAAC
AGCGGACACGUCAGCAGGCGUUGAGGUGCUGAGCUGUGGAUGCAGAACCA
GCGCCAUGCUGAAGAGCCUCAGCCACCUCCUGUCCUUUGGGAUUCGUUUU
UCCCGCUUUCUCUUUGUUUGUCGUCUCAGAAUCUGUGACACAAAGAAACC
CAUCUCCUGUCUUAGGAAACCUAAUGCUGCAAACUCUACCUAGAGGAACC
UUUGAAGACUGUUACAUAAGAACAUACCUUCCUCAGAAGAGGAGUUUCCU
CUGCCCUCUGCCCUUCUCCCCUGCCUCCCUCCCUCCCCUCCUUUUAUUUU
GUUUUAGUGAGCUUAAGAAACAGCAGAUGUGUCUUUCACGGAUCUAACGG
GUGUUGUCCUGAUCGAGAAAAAAACUGGGAUGAGAAUGGUUUGGACUGGA
GUUGGAAGGGGAGGACGGUACUGGGGGUAGGGUUUGGAACAGAGCUACAC
UGGACUCGGGCACAUUCGGAGCAGCAUCCUUUAGUAUGGAGGCUACUUCU
CAGGUAACCAGGAAUUGAGGGGAAGGACCUUGUGGAGGCCGAGCAUUAAC
AGCAAGAGCGGGGUUUGGAGAAAGUCUGAGAUUGGGUGCAGCCCUGACUU
ACCUGCUGGCCCUGACCAGUUUCUUUUCACUAACUUGGCCUUGGGCAUAG
GAUGAAACAUUUUUUCUGCCCUAAUUUUAAAACUAGGUGAGGGUAGAAUC
AUCACAGGUUAGGAAUACAUUCUUCAUAAGACACGAUGCUGUAAAUACCC
UUAAUGGACGAAAAGUUGAAAUACUUUUGUUUCCUCUUGGAGCAGUUCAG
GGAAAUGCCCACAGGGGAUUGUCCUGCACAGAUAGGGCAAGAGGAUUUCC
UGGGUGGAGUCUGCCAAGGCCUGCCUCGCUGGGGACCCCAGAGUCCUGCA
CCUCUGGUUCCGCCCCAGGUGGUGACAUUACUGUCCCCGUUCUGUGGCUC
GUGGACAAGACUUUCUCCAGACCCCUUAAAGUGGUACAUAUUCUAAAAAA
CUGUUUUUCUAUUAUGCCAUAACCUUGCUCUAGUCAGUGAAUGUUCCUAA
UGCUGCUGUUUCAACAUUUGAAUUCUUUUUAAUUUAUGAAACAUGCUAAA
UUUUUUUUUUCAAACAAAACACACACAUCCACAUAUACACAUGCUUCGCU
AUGUGGCUUCCAAGGUUUAAAUUUUGAAAAGUAAAAGAAUUAAAACUUCA
CGACCACAGAUCACCUCAAACCAGAAAUACCUCAGAAUUUUCUACUUAUG
UAAGGUUUAUUAUAUAUUUUGUUAGUUGUGUUGUCUUGUAGUAAGUAUAU
UUUAAUGUAAGUUGGCUUUUGUGACAAGGAAGUUUAAAAGAAAUAGAGAA
AAAGAAAAAAGUUUGCAUCUUCUAGGGAGUGCUACCAUUUUUGUUUGAUA
ACGCCCCCUUGUAAAUAAUUGUCAUCAACUGUAGGUUGGCUGUCUGGGCC
AAGUCUGGGCAUUUAUCAGUCUUGUUUGUGAAGGCUUUUCCUUCUGGUUU
CUUUAGAUCAUUUUAUUUAAAAACAGUGCAUCUCUUCAUCGUGAGGGUAG
GCAAGGCGGGGGCCGUGGGGAGAGGUUGACCUGGGUGAGAACUGAAGAGG
CCGCCUCCUCUUGGGUUGUUUGGAGCUUCACAUGUAAUUCACAUGUAACA
UGUAACUUGAUCGGUCAGUGUUCAGAAUGACAAGUAACCCCGCUUAAACU
UGGUAGAAGGAUGGCCCUUAGACCUGAAUGGGGUGAUUUUACUUGGGAUU
UAACUUCUUCAGCAAAUUAACAGCAACGUUGGAAGAGAUCUGUGGCGCCU
CUGUGAAGCACACCGUGACUCAGGCCAGUCUUUUAGUGCAGCGUGUCUGG
GAGUGAAGGGUUUUGCCCUUGCUGGUCUUGGAGUCCACAGUGUGAGGGGC
ACUGCACAUGCCUGGGCAUCUACCUAGUGUGCUAUGUUCAGUGUCUGGGG
CUUACUGCCCCGGGGUCCUUUCCUCUGGGUGUUGGGGCACAGGGUGCUAU
GGGAGGCCCAUUUGCUUCCCUCUCGGAGCUCAGUUUUUGCUUCAUGGGUC
AAAAUGUGGGCUGGCCAAGUGGUUACAGGAACAGGGUUUCGGUAAGCUAU
GUUGUCUUUUUUUUUUUUUUUUUUUUUUUUUUUAAUGGUUUGAUUUUGUG
CUGUGGUAUUUUUUUUCCCUUAGAAUAAUUUUUAAUGGCAAAACAGGCCU
UACAGCAGUUGCUUUUCUUUACCAUUUAUUUCUUUAAGAAGCUUUAAAAU
AUUUAUUGAAAAGUGCCAUAUCUAAUUUCUUUAGCUUUCGCCUCAGGCAG
UGCAGGCAUCUUUACUUUUCAUCCUCAGAAGAAACAAACGACUAACAAAU
GUAGCAAAUUUACUGCAGGAAUAGUUAGGUCAUGAUACUACCUGAACACU
AAACCCCAGCCUCUUUGUUUGGUUUUAGUUCCUCUGGGUGGUUUUUCUUU
UGUGUGCUGGCUUGAUUCUUGUGAGAAGUUUUGACCUGGCCAAGGGAGGG
UUGAGCCAUGGUUCUGGUGUGGGACUUUGCGGUCAAGACACAGUACAGAC
AGGUCAGGCCUGCGUGCCUUUUCUCUGGGUGGCCUCCCCGUUAGGCCCAC
CGUACGCUCAGCCACUAUAGUGUCCCUGUGGGGCCUUGCCAUCAGAUUGU
GUGUCAGGAGAUGGUACCUUUUUGGUGUGGCUGGGGAGGAGUGUGGUCCA
UGCCAGUUCUUUGGGCUUCAGGCCACUCUUCCCCUCAUGCUGUGGUGUAA
AGUGCACCCAUCAGGUGGUAUAUCUGGUUCUGAUGGCAAGAAGAAGGUGG
GGGAUCUCCUUAUAGGGCAUGGGUCUAGGAGCACAGAUGGGCCUUUUGCC
CCGGGUAAAUGCUUGUCUGUUUGCUGUCAUGUGUUCUUUGAGGAGUGAGC
CAUCUCGAGCCCUGCUUUGAAUUUACUGGGUCAUAGAGCCUCUGCCUGUG
CUCUUUUCCAUAAUGACUUCAUGUGACAUGCACUUUUGGUGGGCUCAGAU
AAUUGGUUUCUUUUUGUUUUUGACCUCAGGCUCUGUGGCAGACUGGGGAA
AAUGGGGCCUGGCAUCAUUUUCCCUGUCAAUGGGAGGGGCUGUUCCAUGC
AGGGUGGGAGGGGACCAAGUUAGCAGAGAGUAGCCAAGGAUCCUUGCUUC
UUCCUUUCUAGUGUGCUGUCAUCCAAGCAGGCUCCUGGCUGUAGGGAUGG
GCCUUGGGGAAGAAUCUUCUUUGAAAGCAUCUAUGAUAACUGAGAAGUCA
UCCCUAGUUGGAGAAAUCCAGUAAUGAGCAGAAGGAGGAAGCAAGUGAGG
ACAGAGGCCAUUGUAUUACAGUGUCACGCAGAGGGCCCUCAAUGAUGGGG
CAUUGGGGAAGGCUGUAGACAUAGUCAUCAGAACAUCCUGGCCUGGCAUA
AGCUGGGUUUUCUCCUGGGACCAUUGGUCCUCAGCAGGAGUUCUUUGCAU
GAGUUGCUCAGGGGCAAGGGCUGCAAGUGGGCUGUGCUUAGGAGAAAGUG
ACACCUGGCAGUGAGGGAAGAUGGUGAGCAUUAUUAGCCUUUGUUGUCCA
GCAUGGCCUUCUUGUCCUGUCUGCUCUGGAGAGGAGCCUGUGGGACCAGU
CCUGCCUGGGGAGGGCAUACCCACACGUGCCAGCUGAUUCUGACUCUGAA
UACAUCAUGUCCGGACUUGGGGGUGUUUCUGCAGAAAAAGGAGGUUGUUU
UUCAGCCUUGAACAUCUUCAGGAGGAUAGAGACUCUUGCUCACAUAUUCU
UAGCAAAGGGAAGGGUCUCUCAUCUCCAGGCCACAGAGAUAGUUCUUCCA
UUGCCCUAAGAGGCUAGGCUAACCCUCUUGACAUAACUUAGACAGCAAAG
CACUUCAUCCUGUAGUUGGGCUCUGUCACCUUUCUCUUCAGUUGGCCACA
UUCUCGUUUCCUCCAUCCUGCUAUGCUUUGUGUGCUCGGGCUGUGUGUGG
GGUUUUUCCCUGGUGGAAGGAAGCCCAGCUGUGUAUUGAAUGUCCUUCAU
GUGUUGUGUGUGGCUCAGAAAGCCUGUCACUUGGCCCCUGUGCUCUGAGC
CGUGAGGGUGGGGAGGUGGCUGUUCCAUUAAAGUGGGAGUAUUGGAUGGC
CCUCUUGAAACUAGAAUUUUGCCUUUUUUAGUAUGCAGUAUAAAGUUUCC
AGCAUCUAUUGGUAACACAAAGAUUUGCUGGUUUUUAAAAUAAUACAGUA
AGCAUAAGUAUGUAAGUUUUUAGAAUUGGUACUAGAAGUUGGACAGCUAG
UUAUUCUCGAGAACUUUAUUUCACUAGAAAAAUAUACUAAUUGGAAAGCA
GUUUCCAGGAGUUAACUCAGUUUAAUUUUCAGUCUCAGUUAUUUUAGCCU
GUUGAGUUUUUGAUGGCACACCUUUGGAGAGAUGGCCACGCCUGAUUCCC
AUUUCAGGGGCAUCAGACCAUACCUUUUUAAGAAGCUCCGUGAAUCUAGU
CAUCUACCCUUCAUCCUGGGCGAACAGCCAAAAAGAGAAGGGGACAAGGU
GUCUUUUUCUCCUUCUCACUGGGGUGACAUGAAUUCUUUUAGUUAAUGGC
UGUUUGCAAAUUCUAAACUAAUGAAAUACUUAGCAGCUAACAUGUUCAAU
CUAGUAAUGAUGAGUUUAAAUCUCAAUUGACAGUAAUGUUUUAGAUAAAC
AGGCCCAGUAAUUCAGUUGAUGAACUGUAUAUCUUCUCAGUCUAGAUUUG
UAAAUGUUUAAUGAAUUCAGGGUUAUAAGCAUAGUUCUUUAAGUAAGAUU
CCAGAUAGUUGAUUUGCAACCAGCAGUCUACCUAUGAAUGUAUCCCAAAC
CUUUAGAAGAUUGGAAAAGAUUUUUGAAAUAAUGAUUUAGUUUUGUAGGA
AAAACACCCCCUUGAAAAUUAAUUCGGUUGACCCAGUAACAUUUUUUAAA
ACAAUUGGUGGCUCCAAAAGGCCUGCCAACAAAGAAAAGUCCAAAUUAUC
UAGUGGGACAUUUUGAAUGUUUUAUGUUUAUUUUGGGUCCACUGUAAACU
UUGGUUCAAAAAAGAAUUUGAAUUUAAAGAAUUUACCAUUAUUUAAAUUA
UUACCAAGUUUUUACAUUUUCAUGAUGGUAUUUUCCAGGUAUGAAUGAAA
CAUGACUUUUUGAUUGUGGUACUUCCUGUAUCCCCUGUAGUGCCAAAACC
AGUGAUACUUUAUUUGCUCCUAUGGCAGCUCAUAGAGGUAACCGAAGUGA
UUUUUCCUCAGUAAUUGAAACACAUAUUCUCUAAAUGCCAAUGUGUGGUG
AUGGGCCCUGCACUGCCUUCAUUUCUCUAGGGCAGUGUCUUUGGAUUGUC
UAGGGCCUAGGUAAUUCUGAGAACUACUGUAAACCAACCACAGGGCACUA
AAGCAAUGUACACACCACUCUUUGUGUGUAUGGAAGGGGUUAUAUAAACC
UGGGCUAUGCUGGACAUCUACAGAAGAGUAUUACAUUCACUUGCAAAGUU
UACAUUUUUGAGCUCACAGUUAUGAAAAAUAUGACCCACAAGUUUUUCAG
GCAGGUGAGGAUGGGUCUUCUUGCAAAUGCAUGAGUUCUGUCUUGAGUCC
UGGGAACUUCUCUGUUGGUUGAGUGUGGGCUCAUUCCCUGACUCUCCUAA
UCAUGUUUGCGUCAGAAUGUUAGCAUUGUAAAUAAAAGAAUAGGUUGUAU
AAUAGAUACACAACACUUGAAACUUUACUUUAAAAAAAUCGAUAGUUCUA
CAUAUAUAUUUAGUUAUAUCACUUGACAGAUUUCUUCUACACAGUGUGGA
GAUUGUUUUAUACCACAGAUUAUUUUUAUAAAGUUAGUGAAUUUGAAUGA
UUUUGUAAUCAGAGCUAAUGAGCUUUACCUUUCAAGAGAAACGUACACUG
GAGCAUGAGUGGUGUGGAACUUUUACUUAGUGUUUAUAUGGAUUCUUGUG
AUACACUGGCAGACUGGAGUCAAUUUGCGGGUCUUUUUUGGCCAAAACUC
CACUUGUGGUUGUGUAGGACAGUGAUAUUCAGCUCAGCUUCUUGUGGAUU
GGGAGGAGAGAGGGCCUGCAAUGUGUUUUACAUUGGUGCUUCCUCCUGAG
AUUUCUGUUGAACAAAGGGUUCUGAGGUCAAAAAUUAGUUUGUAAGCCUU
UGCCAUAGGACAUAGUCAUGUGAGAGUGUUUGGGGGAACAGAAAUUGUAU
AGGGGUGCCUAUUGGGGUGGGAUGGGACUCGAAUAAGAUUCAGGUACAAA
AACUUUGAAAUGAGAAUCUGGUGGUUUGAGUAAUCCACCAGACUGAAUUA
UCUAAGAUCACAUUAUCCAGGUUGGGGGGCAGAAUUACCCAGUUAAGUAA
UUGUUCAGAAAAGUGGGGAGGGUGGCAUGUGGAUGCAGUGAUCCAAUUAA
AUGGAGAGCUGCCAGGCACAUUUUGUCCUCUCUGGUCAGUGAGAAUGGUU
GGGUUGGCUCGCUGCUUCAAUCUGUGGAAUCAGCCAGGAGCCCAGUGAGG
AAGCUCAGAACCCCAGUAACAGCAGAGCAUCUUUCAGAUAGCUCCAGAGU
UUUCCUGCUUUUCUGAGGAAGCUCAGCAUCACUGCCACAAUACGGAAAGU
GGUCUUCAUUUUAGCCUAUUUAUUUUUAGGCAGAGAGUGGAUGGUUAUUU
GUGUGGGACUUUUGGUGGCGAUAUAUAAUGAAUAAUUAAGUUAAUUUCUG
GUAUGCAUAAUGGCCAGUCCUGAGGCCCAGCUGAAGACCUGUCCCCCAGA
CCCUGCCCGCUGGCUUCAGGCUGCUGCUUCUAGACAGAGGUGCACUGGAC
GGGAUAGUUUUAUCAAGAGAAUCCCUAAUGUGUCAUUUUAAACCAGCUGU
GCUUUUUAUUCAUUCUGGUUGAGCGUAUAGGUUUACACUUUACCCUUUUU
AUACUUGGAAUAAAUUUAGUUCCAGCAGAUCUAGUAGCACUCCAGAAACC
AACCCCAUCUGUUCCCCAUAAAAAGAACAUUUUCUCUGCUCUCCAGCCAC
GUGUCUUGGAAUGUAAUUCUGUUGUGCCUUUGUUUUUAUCACUCUCUUCG
CCCCAAAAGCAACUGCUGUAAGCUUUUUUCUACUUGUCUUUUCUAGUCCC
CAACCUCUACCUUUUUCCUUUUUCCCAGCCCUAAUUUCUGGAUGCACUUC
UGUGAUCCAGGUAUUUUAAGAACCAGUUACCUCAGACCUCAUGUUGAACA
GUGUCGCCAUCUGGGUCCUCUUGAUACUGCAGACUUUUAACGUACACAUG
CAGGAACCCUGCUGAGCGUGGGCACUUGUUUUAAAGCAAAACUCUUCCCA
AGGACUGAAGAAAGGGCUUCUGGCAAGCUCGUCAUGGCAUUGUGGUGGGA
UGGGUCUAGAGUGUCAUCUGAAUGGUGCUUCCUGUGUUCCUCUUUGAAUU
CUGCCAUUUUCAGUAUUCUUGUGUGUCUGAAUAGGCAAAGCGAUUUAAUU
GGCUGGUCUUGCACGCAAAUUAGUUCCAAAGAUAAGCUCUUUGUAACACA
UUUCCAGUCGCUAAUGCUCAAAUGUAGAACAUUCCUUUAAAUGGCAGGAU
AAAAAACCCACUAUCCACCAUAGUGCAUUUUGGGAAGAUGUCUGUAGCAU
AUGUUGCUGUGAAAUUAGGCCUUGUGGGAUAUGGCUGUUUGUCAUUUUGA
UGUAUUUUAAAUAAAUAUAUAUAUUUUUUAAAGAGCCUUUUUUACCAGUU
CAAAAAGUUUAAUUAACCAGCAGUCACCGCAUCUGAAUUUUUGUCUCUGG
GGCAUAGAUGGCAGACCAAGAUUAAAAGUGGUAACUCAGCUAUACGAGCA
UGGGCUACCUUCCUGGGCUCUCCUGCAGUCCUGUAGACCUGCUGUUCCGC
AGACCAUGGGACACAAGGUCAGUGUGUUCCCAGUGAGGGUCCCAAGUCAG
UCAUCUUAAGUGUUUGUUCUCUGCCCCAUUCAGUGGACUGUUGACUUCAG
UCCCUGCAAGUGCUUUAGCCCGAGUGGGGUUUUCUCAGAGCACUGCCACG
AGUUAAGUGUGUGUUUAGCCAAAUAAUUUCUCCGUAAGGGAAAAAUGCAG
UCACCCAAAUUUUACCAACAAUGACAGAGAUGAGAGUAGAAAAGAUUAGG
CAACAUCUGAGUUUUAACUUGAAAAGUGUCCAAGUCAUCAUGAAAGGCCG
ACUGGGAGCAAGUGAUUAUUAGAGAUUCUUCAGGAGACCUCAUCUGAAAA
UGUUAAGACUGCCAGUGAGGGAAGGAAUUGUUAAAAUGCCAGCGGCUUUU
UUUUCCUCUUUUUUUCUGUAAUUCUGUAAAAAUGCAGAGAAAGUUGAGUG
GUACUUCAGAAUUGAGGGAGAGGGUUACCGCAGAGUAGAAAUAUAUUUCU
AGAUUUCAGUUCCACACCACAAAUCCACAACAAUGCCAUUUUUCAACUGU
ACAAAAAUCUGCUUAUGAACUGGACAUGAUCUUAAUGGUAGUGUCAAAGG
CCAAGUUUUUCACCUGUUAAUAUUUUUCCACAUUUGUCCUUGAAUCUGAA
UAACUUUAUACAGUACUGUAAAUUUAACUUACAUCGAGUUUGUUGUCAAU
UCUUAUGAAAAGAGCUUUCUGCAUGUAACACAUACGGUUAAAGAACACAG
CAAAGGACAAAAUUUGCAGGAACAGUUUUGGAACCAACAGAAAAUGUCAC
CUUUUAUUUGCCAUCUUAUAUAUAUCUAUCAGUUUUACCAGCUACUUCUA
AAUUUGUACAUUAUUUGUAAGGGAAAGAAGGAAAACCCUAAGACUUGUCU
AACUUAGUGGAGAAUGUGUGUGUUGGGCUUAGGAUGGAUAGCUAAGUCUU
AUUGAGCUGUGUUACCUAACUUGUAUAUAAAAAUUGUAAUUAAAAGUUUG
GGUUCACCUGUUUCUCACAGUUUAAAAUGAUGAGUAAUUGCAAACUCUGG
AAAUGUGACUAGUAUAUGAUUUAAGGCUGUAGAAGCAAGGAAGCUCUUUC
AAGUGCUAAAACUAAAGACUUCUAGUUUUUGGCUCAAAUAAGUACUGUUU
GUAUACCAGGAUAUGUGAGAUGUAAAUGUAGUAGGUCACUUUUCACCCUU
GUAGCUAUAAAAUAAAAAUUUUGUAGAACAGAAAUAGCUUGUACUACUGA
AUUAACAAAAGUUAUACUAAAGUAUCAUGUUUAAAAAAAAUAUAUAUAUA
UAUACAGAGUUAAGCUUGUUGCUGUUACCCUGUCUGGAUUUGAAAAGUGU
GCUGAUUUAUAUAUAUAUAUUACACACACACACACACACACACACACACA
CACACACACACACACACACACACACACACACACACACACACACAUACACC
UAAAAUGGCCUAAAGCAGACAUCCAUGUAAUUACAGUUGCAAAAUGAAAA
CAUUUUGGAAAGAACAUUGUAUCAUAGUUCAUUCAUUUGCAGUGGAUCUU
UGUUCCUUUUUACUGUGGUAAUUUUAGAAAUGAGUGUCAAGUUUGAAAUU
AGAUCUGCUAAGUUGGGGUUUUGCUGCUUGAACUCUGCACUGGGUCCUCA
AAUAAACCGAUGUGAAUGUAGUUUUUUCCCCCUGUGUGAAGAAGCAGUUA
CACCCCAACAAUAGGAGGAAAAAUCUAGAACUAUUUCAAGUUUUAUCUUU
UUGUAUAUGAAAAUAAAAUAAUAAUAAAACAA
SEQ ID NO: 9:
AUGUACCGGCAUCGCAAGCCCCCCUACGGUCAUGUGGACAUCCAUGAGGA
CCCUGGGCCUCCACCACCAUCCCCUCUGGUGGGCCUGAAGCCACUGCAGC
UGCUGGAGAUCAAGGCUCGGGGGCGCUUUGGCUGUGUCUGGAAGGCCCAG
CUCAUGAAUGACUUUGUAGCUGUCAAGAUCUUCCCACUCCAGGACAAGCA
GUCGUGGCAGAGUGAACGGGAGAUCUUCAGCACACCUGGCAUGAAGCACG
AGAACCUGCUACAGUUCAUUGCUGCCGAGAAGCGAGGCUCCAACCUCGAA
GUAGAGCUGUGGCUCAUCACGGCCUUCCAUGACAAGGGCUCCCUCACGGA
UUACCUCAAGGGGAACAUCAUCACAUGGAACGAACUGUGUCAUGUAGCAG
AGACGAUGUCACGAGGCCUCUCAUACCUGCAUGAGGAUGUGCCCUGGUGC
CGUGGCGAGGGCCACAAGCCGUCUAUUGCCCACAGGGACUUUAAAAGUAA
GAAUGUAUUGCUGAAGAGCGACCUCACAGCCGUGCUGGCUGACUUUGGCU
UGGCUGUUCGAUUUGAGCCAGGGAAACCUCCAGGGGACACCCACGGACAG
GUAGGCACGAGACGGUACAUGGCUCCUGAGGUGCUCGAGGGAGCCAUCAA
CUUCCAGAGAGAUGCCUUCCUGCGCAUUGACAUGUAUGCCAUGGGGUUGG
UGCUGUGGGAGCUUGUGUCUCGCUGCAAGGCUGCAGACGGACCCGUGGAU
GAGUACAUGCUGCCCUUUGAGGAAGAGAUUGGCCAGCACCCUUCGUUGGA
GGAGCUGCAGGAGGUGGUGGUGCACAAGAAGAUGAGGCCCACCAUUAAAG
AUCACUGGUUGAAACACCCGGGCCUGGCCCAGCUUUGUGUGACCAUCGAG
GAGUGCUGGGACCAUGAUGCAGAGGCUCGCUUGUCCGCGGGCUGUGUGGA
GGAGCGGGUGUCCCUGAUUCGGAGGUCGGUCAACGGCACUACCUCGGACU
GUCUCGUUUCCCUGGUGACCUCUGUCACCAAUGUGGACCUGCCCCCUAAA
GAGUCAAGCAUCUAA
SEQ ID NO: 10.
ATGACGGCGCCCTGGGTGGCCCTCGCCCTCCTCTGGGGATCGCTGTGCGC
CGGCTCTGGGCGTGGGGAGGCTGAGACACGGGAGTGCATCTACTACAACG
CCAACTGGGAGCTGGAGCGCACCAACCAGAGCGGCCTGGAGCGCTGCGAA
GGCGAGCAGGACAAGCGGCTGCACTGCTACGCCTCCTGGCGCAACAGCTC
TGGCACCATCGAGCTCGTGAAGAAGGGCTGCTGGCTAGATGACTTCAACT
GCTACGATAGGCAGGAGTGTGTGGCCACTGAGGAGAACCCCCAGGTGTAC
TTCTGCTGCTGTGAAGGCAACTTCTGCAACGAACGCTTCACTCATTTGCC
AGAGGCTGGGGGCCCGGAAGTCACGTACGAGCCACCCCCGACAGCCCCCA
CCCTGCTCACGGTGCTGGCCTACTCACTGCTGCCCATCGGGGGCCTTTCC
CTCATCGTCCTGCTGGCCTTTTGGATGTACCGGCATCGCAAGCCCCCCTA
CGGTCATGTGGACATCCATGAGGACCCTGGGCCTCCACCACCATCCCCTC
TGGTGGGCCTGAAGCCACTGCAGCTGCTGGAGATCAAGGCTCGGGGGCGC
TTTGGCTGTGTCTGGAAGGCCCAGCTCATGAATGACTTTGTAGCTGTCAA
GATCTTCCCACTCCAGGACAAGCAGTCGTGGCAGAGTGAACGGGAGATCT
TCAGCACACCTGGCATGAAGCACGAGAACCTGCTACAGTTCATTGCTGCC
GAGAAGCGAGGCTCCAACCTCGAAGTAGAGCTGTGGCTCATCACGGCCTT
CCATGACAAGGGCTCCCTCACGGATTACCTCAAGGGGAACATCATCACAT
GGAACGAACTGTGTCATGTAGCAGAGACGATGTCACGAGGCCTCTCATAC
CTGCATGAGGATGTGCCCTGGTGCCGTGGCGAGGGCCACAAGCCGTCTAT
TGCCCACAGGGACTTTAAAAGTAAGAATGTATTGCTGAAGAGCGACCTCA
CAGCCGTGCTGGCTGACTTTGGCTTGGCTGTTCGATTTGAGCCAGGGAAA
CCTCCAGGGGACACCCACGGACAGGTAGGCACGAGACGGTACATGGCTCC
TGAGGTGCTCGAGGGAGCCATCAACTTCCAGAGAGATGCCTTCCTGCGCA
TTGACATGTATGCCATGGGGTTGGTGCTGTGGGAGCTTGTGTCTCGCTGC
AAGGCTGCAGACGGACCCGTGGATGAGTACATGCTGCCCTTTGAGGAAGA
GATTGGCCAGCACCCTTCGTTGGAGGAGCTGCAGGAGGTGGTGGTGCACA
AGAAGATGAGGCCCACCATTAAAGATCACTGGTTGAAACACCCGGGCCTG
GCCCAGCTTTGTGTGACCATCGAGGAGTGCTGGGACCATGATGCAGAGGC
TCGCTTGTCCGCGGGCTGTGTGGAGGAGCGGGTGTCCCTGATTCGGAGGT
CGGTCAACGGCACTACCTCGGACTGTCTCGTTTCCCTGGTGACCTCTGTC
ACCAATGTGGACCTGCCCCCTAAAGAGTCAAGCATCTAA
SEQ ID NO: 11.
Met Thr Ala Pro Trp Val Ala Leu Ala Leu Leu Trp
Gly Ser Leu Cys Ala Gly Ser Gly Arg Gly Glu Ala
Glu Thr Arg Glu Cys Ile Tyr Tyr Asn Ala Asn Trp
Glu Leu Glu Arg Thr Asn Gln Ser Gly Leu Glu Arg
Cys Glu Gly Glu Gln Asp Lys Arg Leu His Cys Tyr
Ala Ser Trp Arg Asn Ser Ser Gly Thr Ile Glu Leu
Val Lys Lys Gly Cys Trp Leu Asp Asp Phe Asn Cys
Tyr Asp Arg Gln Glu Cys Val Ala Thr Glu Glu Asn
Pro Gln Val Tyr Phe Cys Cys Cys Glu Gly Asn Phe
Cys Asn Glu Arg Phe Thr His Leu Pro Glu Ala Gly
Gly Pro Glu Val Thr Tyr Glu Pro Pro Pro Thr Ala
Pro Thr Leu Leu Thr Val Leu Ala Tyr Ser Leu Leu
Pro Ile Gly Gly Leu Ser Leu Ile Val Leu Leu Ala
Phe Trp Met Tyr Arg His Arg Lys Pro Pro Tyr Gly
His Val Asp Ile His Glu Asp Pro Gly Pro Pro Pro
Pro Ser Pro Leu Val Gly Leu Lys Pro Leu Gln Leu
Leu Glu Ile Lys Ala Arg Gly Arg Phe Gly Cys Val
Trp Lys Ala Gln Leu Met Asn Asp Phe Val Ala Val
Lys Ile Phe Pro Leu Gln Asp Lys Gln Ser Trp Gln
Ser Glu Arg Glu Ile Phe Ser Thr Pro Gly Met Lys
His Glu Asn Leu Leu Gln Phe Ile Ala Ala Glu Lys
Arg Gly Ser Asn Leu Glu Val Glu Leu Trp Leu Ile
Thr Ala Phe His Asp Lys Gly Ser Leu Thr Asp Tyr
Leu Lys Gly Asn Ile Ile Thr Trp Asn Glu Leu Cys
His Val Ala Glu Thr Met Ser Arg Gly Leu Ser Tyr
Leu His Glu Asp Val Pro Trp Cys Arg Gly Glu Gly
His Lys Pro Ser Ile Ala His Arg Asp Phe Lys Ser
Lys Asn Val Leu Leu Lys Ser Asp Leu Thr Ala Val
Leu Ala Asp Phe Gly Leu Ala Val Arg Phe Glu Pro
Gly Lys Pro Pro Gly Asp Thr His Gly Gln Val Gly
Thr Arg Arg Tyr Met Ala Pro Glu Val Leu Glu Gly
Ala Ile Asn Phe Gln Arg Asp Ala Phe Leu Arg Ile
Asp Met Tyr Ala Met Gly Leu Val Leu Trp Glu Leu
Val Ser Arg Cys Lys Ala Ala Asp Gly Pro Val Asp
Glu Tyr Met Leu Pro Phe Glu Glu Glu Ile Gly Gln
His Pro Ser Leu Glu Glu Leu Gln Glu Val Val Val
His Lys Lys Met Arg Pro Thr Ile Lys Asp His Trp
Leu Lys His Pro Gly Leu Ala Gln Leu Cys Val Thr
Ile Glu Glu Cys Trp Asp His Asp Ala Glu Ala Arg
Leu Ser Ala Gly Cys Val Glu Glu Arg Val Ser Leu
Ile Arg Arg Ser Val Asn Gly Thr Thr Ser Asp Cys
Leu Val Ser Leu Val Thr Ser Val Thr Asn Val Asp
Leu Pro Pro Lys Glu Ser Ser Ile

1. A compound that is capable of allowing a target cell to produce a mutant activin receptor type-2B (ACVR2B) mRNA where a part of the sequence that encodes some or all of the intracellular region of wild-type ACVR2B is absent, or a pharmaceutically acceptable salt or hydrate thereof.

2. The compound or pharmaceutically acceptable salt or hydrate thereof according to claim 1, wherein said intracellular region of wild-type ACVR2B is encoded by exons 5 to 11 of wild-type ACVR2B.

3. The compound or pharmaceutically acceptable salt or hydrate thereof according to claim 1, which is capable of making the target cell produce a truncated ACVR2B protein that lacks part of the intracellular region of wild-type ACVR2B.

4. The compound or pharmaceutically acceptable salt or hydrate thereof according to claim 3, wherein the truncated ACVR2B protein lacks all or part of the intracellular region encoded by at least one exon selected from the group consisting of exons 5, 6, 7, 8, 9 and 10 of ACVR2B.

5. The compound according to claim 1, which is an antisense oligomer capable of inducing the skipping of an exon coding for a part of intracellular region of ACVR2B, or a pharmaceutically acceptable salt or hydrate thereof.

6. The compound or pharmaceutically acceptable salt or hydrate thereof according to claim 5, wherein said exon to be skipped is selected from the group consisting of exons 5, 6, 7, 8, 9 and 10 of ACVR2B.

7. The compound according to claim 5, which comprises 10-50 nucleobases, or pharmaceutically acceptable salt or hydrate thereof.

8. The compound according to claim 5, comprising a sequence complementary to 10 to 50 consecutive nucleotides of an exon selected from the group consisting of exons 5, 6, 7, 8, 9 and 10 of ACVR2B, or pharmaceutically acceptable salt or hydrate thereof.

9. The compound or pharmaceutically acceptable salt or hydrate thereof according to claim 5, wherein the exon comprises a sequence selected from the group consisting of SEQ ID NOs: 1 to 6.

10. The compound according to claim 5, comprising a nucleotide sequence selected from the group consisting of SEQ ID NOs: 12 to 36 and 43 to 111, or pharmaceutically acceptable salt or hydrate thereof.

11. The compound according to claim 5, consisting of a nucleotide sequence selected from the group consisting of SEQ ID NOs: 12 to 36 and 43 to 111, or pharmaceutically acceptable salt or hydrate thereof.

12. The compound or pharmaceutically acceptable salt or hydrate thereof according to claim 5, wherein the antisense oligomer is an oligonucleotide.

13. The compound or pharmaceutically acceptable salt or hydrate thereof according to claim 12, wherein at least one sugar moiety and/or at least one phosphate bond moiety in the oligonucleotide is modified.

14. The compound or pharmaceutically acceptable salt or hydrate thereof according to claim 13, wherein the modified sugar moiety is a ribose in which the —OH group at the 2′-position is substituted with any group selected from the group consisting of OR, R, R′OR, SH, SR, NH₂, NHR, NR₂, N₃, CN, F, Cl, Br and I (wherein R represents alkyl or aryl, and R′ represents alkylene).

15. The compound or pharmaceutically acceptable salt or hydrate thereof according to claim 13, wherein the modified phosphate bond moiety is one selected from the group consisting of a phosphorothioate bond, a phosphorodithioate bond, an alkylphosphonate bond, a phosphoroamidate bond and a boranophosphate bond.

16. The compound or pharmaceutically acceptable salt or hydrate thereof according to claim 5, wherein the antisense oligomer comprises at least one morpholino ring.

17. The compound according to claim 16, which is a morpholino oligomer or phosphorodiamidate morpholino oligomer, or pharmaceutically acceptable salt or hydrate thereof.

18. The compound according to claim 16, having any one of the groups represented by chemical formulae (1) to (3) shown below at its 5′-terminal end, or pharmaceutically acceptable salt or hydrate thereof.

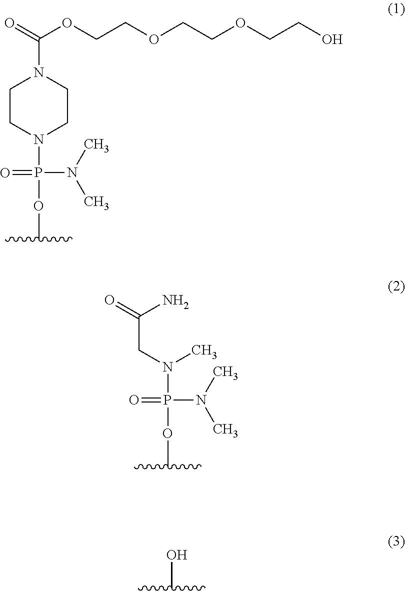

19. A compound which is a conjugate wherein a cell penetrating peptide is bonded to the compound according to claim 1, or pharmaceutically acceptable salt or hydrate thereof.

20. A pharmaceutical composition comprising the compound or pharmaceutically acceptable salt or hydrate thereof according to claim 1.

21. The pharmaceutical composition according to claim 20, which further comprises at least one pharmaceutically acceptable carrier or additive.

22. The pharmaceutical composition according to claim 20, which is lyophilized.

23-26. (canceled)

27. A method for treating an amyotrophic disease, a muscle wasting disease or a sarcopenic disease in a subject, which comprises administering to said subject a therapeutically effective amount of the compound or pharmaceutically acceptable salt or hydrate thereof according to claim 1 or a pharmaceutical composition comprising the compound or pharmaceutically acceptable salt or hydrate thereof according to claim 1.

28. The method according to claim 27, wherein the amyotrophic disease is Duchenne muscular dystrophy.

29. The method according to claim 27, wherein the subject is a human.

30-32. (canceled)

33. A genetically manipulated animal that expresses a mutant activin receptor type-2B (ACVR2B) mRNA where a part of the sequence that encodes some or all of the intracellular region of wild-type ACVR2B is absent.
